SEQUENCE LISTING (3 sequences)

SEQ ID NO: 1 - DNA, 21 bp, Artificial Sequence
  Synthetic
gcgtttgctcttcttcttgcg

SEQ ID NO: 2 - DNA, 43 bp, Artificial Sequence
  Synthetic
aatgatacggcgaccaccgagatctacactctttccctacacg

SEQ ID NO: 3 - DNA, 45 bp, Artificial Sequence
  Synthetic
caagcagaagacggcatacgagatgtcgtgatgtgactggagttc

(19) United States
(12) Patent Application Publication
(10) Pub. No.: US 2022/0017471 A1
(43) Pub. Date: Jan. 20, 2022

(54) FUNCTIONALITY INDEPENDENT LABELING OF ORGANIC COMPOUNDS

(71) Applicant: Shanghai Tech University, Shanghai (CN)

(72) Inventors: Guang Yang, Shanghai (CN); Richard A. Lerner, Shanghai (CN); Biao Jiang, Shanghai (CN); Peixiang Ma, Shanghai (CN); Hongtao Xu, Shanghai (CN)

(21) Appl. No.: 17/260,992

(22) PCT Filed: Jul. 17, 2019
(86) PCT No.: PCT/CN2019/096388

§ 371 Date: Jan. 15, 2021

(30) Foreign Application Priority Data
Jul. 18, 2018 (CN) PCT/CN2018/096052
Apr. 24, 2019 (CN) PCT/CN2019/084031

(51) Int. Cl. C07D 229/02; C40B 80/00; C40B 70/00; C40B 30/04

(52) U.S. Cl. CPC C07D 229/02; C40B 30/04; C40B 70/00; C40B 80/00

(57) ABSTRACT

Disclosed herein are methods of labeling organic compounds without depending on any functional group of the compound. In some embodiments, provided are bifunctional linkers useful in the methods.

### CROSS REFERENCE TO RELATED APPLICATIONS

The present invention is a national stage application of PCT application PCT/CN2019/096388, filed on Jul. 17, 2019, which claims the priority of the PCT/CN2018/096052, filed on Jul. 18, 2018 and the PCT/CN2019/084031, filed on Apr. 24, 2019, the contents of which are incorporated herein by its entirety.

### SEQUENCE LISTING

The instant application contains a Sequence Listing which has been submitted in ASCII format via EFS-Web and is hereby incorporated by reference in its entirety. Said ASCII copy, created on Jul. 16, 2021, is named 264299_ST25.txt and is 4,096 bytes in size.

### FIELD

This disclosure generally relates to chemical and biological, especially to the natural product labeling and screening.

### BACKGROUND

Currently, target identification for the natural products typically requires a lot of work for structure-activity relationship (SAR) in order to introduce a proper tag, such as affinity tag and cross-link tag. Such work is usually a trial-error procedure. Tags, if on the improper sites, will introduce spatial hindrance for the interaction of the compound and the target and cause false-negative results in the biological screening. Therefore, the target identification needs various drug-tag conjugations with different site labeling to reduce the false-negative results. However, the multiple sites labeling depends on the functional group on the natural product or total synthesis to introduce new functional group. The structure of natural products are complicated and the total synthesis are challenging for the chemists. So such approaches are time-consuming and inefficient.

With the development of chemistry, more and more novel natural products are isolated. In order to high-throughput screen the chemicals, several agencies proposed strategies using combinatorial chemistry and DNA-encoding technology, see, for example, U.S. Pat. No. 5,565,324, EP0643778, U.S. Pat. No. 7,935,658, WO/2010/094036, and CN103882531.

These approaches use combinatorial chemistry to establish the chemical library, using the small-chemical fragment to build large chemicals. But these chemical reactions are limited by the DNA stability, so many common reagents, including strong base, strong reducing reagents etc., are excluded. Therefore many complicated natural products are not included in the combinatorial chemical library.

### SUMMARY

This disclosure provides a novel labeling strategy to cover a larger chemical space. Accordingly, provided herein are methods for site non-selective labeling of organic chemicals using oligonucleotides, compounds useful in the methods, as well as the labeled compounds, libraries comprising the labeled compounds and uses thereof.

In some embodiments, provided is a method for labeling an organic compound, which method comprises:

(1) contacting a linker precursor molecule with an organic compound under site non-selective reaction conditions, e.g., carbene or nitrene or free-radical reaction conditions, wherein the linker precursor molecule has two functional groups, a first functional group R and a second functional group M, wherein the first functional group R of the linker precursor molecule generates a site non-selective reacting group, e.g., carbene or nitrene or free-radical, under the site non-selective reaction conditions that reacts with the organic compound, the second functional group is unreactive under the conditions when the first functional group reacts, and wherein contacting of the linker precursor molecule with the organic compound forms an intermediate having the second functional group M of the linker precursor molecule; and

(2) contacting the intermediate of (1) with a labeling molecule thereby the second functional group M of the linker precursor molecule reacts with the labeling molecule to form the labeled organic compound, wherein the labeling molecule comprises a label.

In some embodiments, provided is a linker precursor molecule comprising a first functional group and a second functional group, wherein the first functional group is capable of generating a site non-selective reacting group, e.g., carbene, nitrene or free-radical, that is capable of reacting with an organic compound in a site non-selective fashion, and the second functional group is unreactive under the conditions when the first functional group reacts with the organic compound, but is capable of reacting with a labeling molecule.

In some embodiments, provided is a labeled organic compound which is produced by a method comprising:

(1) contacting a linker precursor molecule with an organic compound under site non-selective reaction conditions, e.g., carbene or nitrene or free-radical reaction conditions, wherein the linker precursor molecule has two functional groups, a first functional group R and a second functional group M, wherein the first functional group R of the linker precursor molecule generates a site non-selective reacting group, e.g., carbene or nitrene or free-radical, under the site non-selective reaction conditions that reacts with the organic compound, the second functional group is unreactive under the conditions when the first functional group reacts with the organic compound, and wherein contacting of the linker precursor molecule with the organic compound forms an intermediate having the second functional group M of the linker precursor molecule; and

(2) contacting the intermediate of (1) with a labeling molecule thereby the second functional group M of the linker precursor molecule reacts with the labeling molecule to form the labeled organic compound, wherein the labeling molecule comprises a label.

In some embodiments, provided is a set of labeled organic compounds comprising positional isomers, each of which comprises a label moiety, a linker, and an organic compound moiety, wherein the isomers differ in the site of the organic compound moiety to which the label moiety is attached through the linker.

In some embodiments, provided is a library of labeled organic compounds comprising at least two labeled organic compounds (or two sets of isomeric labeled organic compounds), wherein a unique organic compound is labeled with a unique label.

In some embodiments, provided is a method of identifying organic compounds that bind to a target, comprising assaying a labeled organic compound, a set of isomeric labeled organic compounds, or a library of labeled organic compounds described herein, and identifying the organic compounds that bind to the target according to their labels.

These and other embodiments will be further described in the text that follows.

### BRIEF DESCRIPTION OF THE DRAWINGS

FIG. 1 shows HPLC and mass spectra of oridonin linker I in Example 5.

FIG. 2 shows a mass spectrum of compound 8 in Example 6.

FIG. 3 shows a mass spectrum of labeled compound 9 in Example 7.

FIG. 4 shows a mass spectrum of labeled compound 8 in Example 8.

FIGS. 5 and 6 show the HPLC and mass spectra of oridonin-linker II conjugate compounds of Example 9.

FIGS. 7 and 8 show the HPLC and mass spectra of celestrol-linker II conjugate compounds of Example 10.

FIGS. 9 and 10 show the HPLC and mass spectra of taxol-linker II conjugate compounds of Example 11.

FIGS. 11 and 12 show the HPLC and mass spectra of triptophenolide-linker II conjugate compounds of Example 12.

FIGS. 13 and 14 show the HPLC and mass spectra of maytansinol-linker II conjugate compounds of Example 13.

FIGS. 15 and 16 show the HPLC and mass spectra of dehydroabietic acid linker II conjugate (A) and hydroabietic acid linker II conjugate (B) compounds of Example 14.

FIG. 17 shows DNA migration in agarose gel electrophoresis of four samples: 1. Compound 6 conjugate with oligonucleotides; 2. Compound 7 conjugate with oligonucleotides; 3. Un-reacted oligonucleotides; and 4. Headpiece DNA 4 with linker II irradiated by UV light for 2 hours, and then ligated with oligonucleotides.

FIG. 18 shows ¹H NMR of the crude irradiation product 2,4-dihydroxyacetophenone labeled with linker IV without vacuum; and ii) crude product of 2,4-dihydroxyacetophenone labeled with linker IV after vacuum. Linker IV-2,4-dihydroxyacetophenone conjugates identified with an arrow.

FIG. 19 shows a general scheme for the quantification of natural product-DNA conjugation by quantitative polymerase-chain-reaction (qPCR) and sequencing.

FIG. 20(a) shows DEL selection fingerprints for heat shock 70 kDa protein (HSP70). FIG. 20(b) shows DEL selection fingerprints for poly [ADP-ribose] polymerase 1 (PARP1). In FIG. 20, the red dashed lines are the cut-off for hits selection. Corresponding chemical structures illustrated in FIG. 21.

FIG. 21 shows the chemical structures of selected binders from the DEL selection for PAPR1.

FIG. 22 shows hit validation of nDEL selected PARP1 binders. Inhibition of enzyme activity of human PARP1 by Luteolin (FIG. 22(a)) and F003 (FIG. 22(b)). Molecular docking of Luteolin in the active site of human PARP1 is shown in FIG. 22(c).

It will be recognized that some or all of the figures are schematic representations for purpose of illustration.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used herein the following definitions apply unless clearly indicated otherwise:

As used herein and in the appended claims, the singular forms “a”, “an”, and “the” include plural referents unless the context clearly dictates otherwise. Thus, for example, reference to “a product” includes a plurality of products, such as isomers.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) claimed. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

“Alkyl” refers to monovalent saturated linear or branched aliphatic hydrocarbyl groups. In some embodiments, alkyl has from 1 to 30 carbon atoms (i.e., C₁₋₃₀ alkenyl), 1 to 20 carbon atoms (i.e., C₁₋₂₀ alkenyl), 1 to 8 carbon atoms (i.e., C₁₋₈ alkenyl), 1 to 6 carbon atoms (C₁₋₆ alkyl), or 1 to 4 carbon atoms (i.e., C₁₋₄ alkenyl). This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), and t-butyl ((CH₃)₃C—). “Alkylene” refers to a divalent linear or branched saturated aliphatic hydrocarbyl group.

“Alkenyl” refers to an alkyl group containing at least one carbon-carbon double bond. In some embodiments, alkenyl has from 2 to 30 carbon atoms (i.e., C₂₋₃₀ alkenyl), 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, but are not limited to, ethenyl, propenyl, butadienyl (including 1,2-butadienyl and 1,3-butadienyl). “Alkenylene” refers to a divalent alkenyl group.

“Alkynyl” refers to an alkyl group containing at least one carbon-carbon triple bond. In some embodiments, alkenyl has from 2 to 30 carbon atoms (i.e., C₂₋₃₀ alkynyl), 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond. “Alkynylene” refers to a divalent alkynyl group.

“Alkoxy” refers to the group “alkyl-O—”. Examples of alkoxy groups include, but are not limited to, methoxy, ethoxy, n-propoxy, iso-propoxy, n-butoxy, tert-butoxy, sec-butoxy, n-pentoxy, n-hexoxy, and 1,2-dimethylbutoxy.

“Haloalkoxy” refers to an alkoxy group as defined above, wherein one or more hydrogen atoms are replaced by a halogen.

“Alkylthio” refers to the group “alkyl-S—”.

“Acyl” refers to a group —C(O)R, wherein R is hydrogen, alkyl, alkenyl, alkynyl, cycloalkyl, heterocycloalkyl, aryl, heteroalkyl, or heteroaryl; each of which may be optionally substituted, as defined herein. Examples of acyl include, but are not limited to, formyl, acetyl, cylcohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently selected from the group consisting of hydrogen, alkyl, alkenyl, alkynyl, cycloalkyl, heterocycloalkyl, aryl, heteroalkyl, or heteroaryl; each of which may be optionally substituted.

“Amino” refers to the group —NR^{y}R^{z} wherein R^{y} and R^{z} are independently selected from the group consisting of hydrogen, alkyl, alkenyl, alkynyl, heteroalkyl, cycloalkyl, heterocycloalkyl, aryl, or heteroaryl; each of which may be optionally substituted.

“Amidino” refers to —C(NH)(NH₂).

“Aryl” refers to a monovalent aromatic carbocyclic group having a single ring (e.g. monocyclic) or multiple rings (e.g. bicyclic or tricyclic) including fused systems. In some embodiments, aryl has 6 to 20 ring carbon atoms (i.e., C₆₋₂₀ aryl), 6 to 14 carbon ring atoms (i.e., C₆₋₁₄ aryl), 6 to 12 carbon ring atoms (i.e., C₆₋₁₂ aryl), or 6 to 10 carbon ring atoms (i.e., C₆₋₁₀ aryl). Examples of aryl groups include, but are not limited to, phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocycloalkyl, the resulting ring system is heterocycloalkyl. “Arylene” refers to a divalent aryl group.

“O-carbamoyl” refers to the group —O—C(O)NR^{y}R^{z} and “N-carbamoyl” group refers to the group —NR^{y}C(O)OR^{z}, wherein R^{y} and R^{z} are independently selected from the group consisting of hydrogen, alkyl, alkenyl, alkynyl, heteroalkyl, cycloalkyl, heterocycloalkyl, aryl, or heteroaryl; each of which may be optionally substituted.

“Carboxyl” refers to —C(O)OH.

“Carboxyl ester” refers to both —OC(O)R and —C(O)OR, wherein R is hydrogen, alkyl, alkenyl, alkynyl, heteroalkyl, cycloalkyl, heterocycloalkyl, aryl, or heteroaryl; each of which may be optionally substituted, as defined herein.

“Cyano” or “carbonitrile” refers to the group —CN.

“Cycloalkyl” refers to a monovalent saturated or partially unsaturated non-aromatic cyclic alkyl group having a single ring or multiple rings including fused, bridged, and spiro ring systems. The term “cycloalkyl” includes cycloalkenyl groups (i.e. the nonaromatic carbocyclic group having at least one double bond). In some embodiments, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include, but are not limited to, cyclopropyl, cyclobutyl, cyclopentyl, and cyclohexyl. “Cycloalkylene” refers to a divalent saturated or partially unsaturated cyclic alkyl group.

“Guanidino” refers to —NHC(NH)(NH₂).

“Hydrazino” refers to —NHNH₂.

“Imino” refers to a group —C(NR)R, wherein each R is alkyl, alkenyl, alkynyl, heteroalkyl, cycloalkyl, heterocycloalkyl, aryl, or heteroaryl; each of which may be optionally substituted, as defined herein.

“Halogen” or “halo” includes fluoro, chloro, bromo, and iodo.

“Haloalkyl” refers to an alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Haloalkenyl” refers to an alkenyl group as defined above, wherein one or more hydrogen atoms are replaced by a halogen.

“Haloalkynyl” refers to an alkynyl group as defined above, wherein one or more hydrogen atoms are replaced by a halogen.

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —S(O)—, —S(O)₂—, and the like, where R is H, alkyl, alkenyl, alkynyl, heteroalkyl, cycloalkyl, heterocycloalkyl, aryl, or heteroaryl, each of which may be optionally substituted. Examples of heteroalkyl groups include —OCH₃, —CH₂OCH₃, —SCH₃, —CH₂SCH₃, —NHCH₃, and —CH₂NRCH₃. In some embodiments, heteroalkyl includes 1 to 10 carbon atoms, 1 to 8 carbon atoms, or 1 to 4 carbon atoms; and 1 to 3 heteroatoms, 1 to 2 heteroatoms, or 1 heteroatom. “Heteroalkylene” refers to a divalent heteroalkyl group.

“Heteroaryl” refers to an aromatic group having a single ring, multiple rings, or multiple fused rings, with one or more ring heteroatoms independently selected from nitrogen, oxygen, and sulfur. In some embodiments, heteroaryl includes 5 to 24 ring atoms (i.e., 5 to 24 membered heteroaryl), or 5 to 14 ring atoms (i.e., 5 to 14 membered heteroaryl), 5 to 10 ring atoms (i.e., 5 to 10 membered heteroaryl), or 5 or 6 ring atoms (i.e., 5 or 6 membered heteroaryl). In some embodiments, heteroaryl includes 1 to 20 ring carbon atoms (i.e., C₁₋₂₀ heteroaryl), 5 to 14 ring carbon atoms (i.e., C₅₋₁₄ heteroaryl), 3 to 12 ring carbon atoms (i.e., C₃₋₁₂ heteroaryl), or 3 to 8 carbon ring atoms (i.e., C₃₋₈ heteroaryl); and 1 to 5 heteroatoms, 1 to 4 heteroatoms, 1 to 3 ring heteroatoms, 1 to 2 ring heteroatoms, or 1 ring heteroatom independently selected from nitrogen, oxygen, and sulfur. Examples of heteroaryl groups include, but are not limited to, pyrimidinyl, purinyl, pyridyl, pyridazinyl, benzothiazolyl, pyrazolyl, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above. “Heteroarylene” refers to a divalent heteroaryl group.

“Heterocycloalkyl” refers to a saturated or unsaturated non-aromatic cyclic alkyl group, with one or more ring heteroatoms independently selected from nitrogen, oxygen and sulfur. The term “heterocycloalkyl” includes heterocycloalkenyl groups (i.e. the heterocycloalkyl group having at least one double bond), bridged-heterocycloalkyl groups, fused-heterocycloalkyl groups, and spiro-heterocycloalkyl groups. A heterocycloalkyl may be a single ring or multiple rings wherein the multiple rings may be fused, bridged, or spiro. Any non-aromatic ring containing at least one heteroatom is considered a heterocycloalkyl, regardless of the attachment (i.e., can be bound through a carbon atom or a heteroatom). Further, the term heterocycloalkyl is intended to encompass any non-aromatic ring containing at least one heteroatom, which ring may be fused to an aryl or heteroaryl ring, regardless of the attachment to the remainder of the molecule. In some embodiments, heterocycloalkyl includes 3 to 24 ring atoms (i.e., 3 to 24 membered heterocycloalkyl), or 3 to 14 ring atoms (i.e., 3 to 14 membered heterocycloalkyl), 3 to 10 ring atoms (i.e., 3 to 10 membered heterocycloalkyl), or 5 or 6 ring atoms (i.e., 5 or 6 membered heterocycloalkyl). In some embodiments, heterocycloalkyl has 2 to 20 ring carbon atoms (i.e., C₂₋₂₀ heterocycloalkyl), 2 to 12 ring carbon atoms (i.e., C₂₋₁₂ heterocycloalkyl), 2 to 10 ring carbon atoms (i.e., C₂₋₁₀ heterocycloalkyl), 2 to 8 ring carbon atoms (i.e., C₂₋₈ heterocycloalkyl), 3 to 12 ring carbon atoms (i.e., C₃₋₁₂ heterocycloalkyl), 3 to 8 ring carbon atoms (i.e., C₃₋₈ heterocycloalkyl), or 3 to 6 ring carbon atoms (i.e., C₃₋₆ heterocycloalkyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocycloalkyl groups include, but are not limited to, pyrrolidinyl, piperidinyl, piperazinyl, oxetanyl, dioxolanyl, azetidinyl, morpholinyl, 2-oxa-7-azaspiro[3.5]nonanyl, 2-oxa-6-azaspiro[3.4]octanyl, 6-oxa-1-azaspiro[3.3]heptanyl, 1,2,3,4-tetrahydroisoquinolinyl, 4,5,6,7-tetrahydrothieno[2,3-c]pyridinyl, indolinyl, and isoindolinyl. “Heterocycloalkylene” refers to a divalent heterocycloalkyl group.

“Hydroxy” or “hydroxyl” refers to the group —OH.

“Oxo” refers to the group (═O) or (O).

“Nitro” refers to the group —NO₂.

“Sulfonyl” refers to the group —S(O)₂R, where R is alkyl, alkenyl, alkynyl, heteroalkyl, cycloalkyl, heterocycloalkyl, aryl, or heteroaryl. Examples of sulfonyl include, but are not limited to, methylsulfonyl, ethylsulfonyl, phenylsulfonyl, and toluenesulfonyl.

“Alkylsulfonyl” refers to the group —S(O)₂R, where R is alkyl.

“Sulfonic acid” refers to the group —SO₃H.

“Alkylsulfinyl” refers to the group —S(O)R, where R is alkyl.

“Thiocyanate” refers to the group —SCN.

“Thiol” refers to the group —SH.

“Thioxo” or “thione” refer to the group (═S) or (S).

Certain commonly used alternative chemical names may be used. For example, a divalent group such as a divalent “alkyl” group, a divalent “aryl” group, etc., may also be referred to as an “alkylene” group, an “arylene” group, respectively. Also, unless indicated explicitly otherwise, where combinations of groups are referred to herein as one moiety, e.g. arylalkyl, the last mentioned group contains the atom by which the moiety is attached to the rest of the molecule.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, N₃, O-carbamoyl, N-carbamoyl, carboxyl, carboxyl ester, cyano, guanidino, halo, haloalkyl, haloalkoxy, heteroalkyl, heteroaryl, heterocycloalkyl, hydroxy, hydrazino, imino, oxo, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. In some embodiments, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted.

As used herein, the term “solvent” refers to a liquid that dissolves a solid, liquid, or gaseous solute to form a solution. Common solvents are well known in the art and include but are not limited to, water; saturated aliphatic hydrocarbons, such as pentane, hexane, heptane, and other light petroleum; aromatic hydrocarbons, such as benzene, toluene, xylene, etc.; halogenated hydrocarbons, such as dichloromethane, chloroform, carbon tetrachloride, etc.; aliphatic alcohols, such as methanol, ethanol, propanol, etc.; ethers, such as diethyl ether, dipropyl ether, dibutyl ether, tetrahydrofuran, dioxane, etc.; ketones, such as acetone, ethyl methyl ketone, etc.; esters, such as methyl acetate, ethyl acetate, etc.; nitrogen-containing solvents, such as dimethylacetamide, formamide, N,N-dimethylformamide, acetonitrile, pyridine, N-methylpyrrolidone, quinoline, nitrobenzene, etc.; sulfur-containing solvents, such as carbon disulfide, dimethyl sulfoxide, sulfolane, etc.; phosphorus-containing solvents, such as hexamethylphosphoric triamide, etc. The term solvent includes a combination of two or more solvents unless clearly indicated otherwise. A particular choice of a suitable solvent will depend on many factors, including the nature of the solvent and the solute to be dissolved and the intended purpose, for example, what chemical reactions will occur in the solution, and is generally known in the art.

As used herein, the term “contacting” refers to bringing two or more chemical molecules to close proximity so that a chemical reaction between the two or more chemical molecules can occur. For example, contacting may comprise mixing and optionally continuously mixing the chemicals. Contacting may be done by fully or partially dissolving or suspending two or more chemicals in one or more solvents, mixing of a chemical in a solvent with another chemical in solid and/or gas phase or being attached on a solid support, such as a resin, or mixing two or more chemicals in gas or solid phase and/or on a solid support, that are generally known to those skilled in the art.

As used herein, “organic compound” refers to a compound that is to be labeled such that is can be screened for binding activity with one or more targets. The terms “drug”, TCM, and natural product (NP) are used to refer to organic compounds to be labeled as defined here.

As used herein, “linker precursor molecule” refers to a molecule having two functional groups each of which reacts and thereby connects with a molecule, such that after the reactions, the residue of the linker precursor molecule becomes of the linker of the product.

As used herein, “labeling molecule” refers to a molecule having a label, such as an oligonucleotide, that can be detected by an analytical method.

As used herein, the term “site non-selective” in “site non-selective reaction,” “site non-selective labeling,” etc., refers to a reaction or labeling etc. that can occur at a number of different sites of a molecule which may not need to depend on the functional group(s) of the molecule. Examples of “site non-selective reaction” include carbene, nitrene and free-radical reactions in which a compound X-Y, wherein Y is a site non-selective reacting group, such as a carbene, nitrene or free-radical group, reacts with different sites of a compound Z to form a compound X-Z. Such site non-selective reactions typically produce several positional isomers X-Z wherein the X is attached to different sites of Z.

As used herein, “isomeric compounds,” or “isomeric products,” etc. refers to isomers produced by a site non-selective reaction, e.g., reacting a linker precursor molecule having a site non-selective reacting group with an organic compound, or the corresponding labeled isomeric products, where appropriate. “A set of isomeric products” refers to the mixture of isomeric compounds produced from one unique organic compound.

As used herein, “disproportionation reaction products” refers to the two products produced by a disproportionation reaction, which is sometimes called dismutation, and is a redox reaction in which a compound of intermediate oxidation state converts to two different compounds, one of higher oxidation state and one of lower oxidation state. A disproportionation reaction can be depicted as:

2P→P′+P″

where P, P′, and P″ are all different chemical species, and P′, and P″ are disproportionation reaction products.

All atoms designated within a formula described herein, either within a structure provided, or within the definitions of variables related to the structure, is intended to include any isotope thereof, unless clearly indicated to the contrary. It is understood that for any given atom, the isotopes may be present essentially in ratios according to their natural occurrence, or one or more particular atoms may be enhanced with respect to one or more isotopes using synthetic methods known to one skilled in the art. Thus, hydrogen includes for example ¹H, ²H, ³H; carbon includes for example ¹¹C, ¹²C, ¹³C, ¹⁴C; oxygen includes for example ¹⁶O, ¹⁷O, ¹⁸O; nitrogen includes for example ¹³N, ¹⁴N, ¹⁵N; sulfur includes for example ³²S, ³³S, ³⁴S, ³⁵S, ³⁶S, ³⁷S, ³⁸S; fluoro includes for example ¹⁷F; ¹⁸F; ¹⁹F; chloro includes for example ³⁵Cl, ³⁶Cl, ³⁷Cl, ³⁸Cl, ³⁹Cl; and the like.

The compounds described herein include any tautomeric forms although the formula of only one of the tautomeric forms of a given compound may be provided herein.

As used herein, the term “salt” refers to acid addition salts and basic addition salts. Examples acid addition salts include those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid. Basic addition salts include those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, Pa., Vol. 2, p. 1457, 1995. Salts include pharmaceutically acceptable salts that do not have properties that would cause a reasonably prudent medical practitioner to avoid administration of the material to a patient, taking into consideration the disease or conditions to be treated and the respective route of administration. The compounds, intermediates or products described herein include their salts.

In addition, abbreviations as used herein have respective meanings as follows:

List of Abbreviations and Acronyms

| | |
|---|---|
| HATU | 1-[bis(dimethylamino)methylene]-1H-1,2,3-triazolo[4,5- |
| | b]pyridinium 3-oxid hexafluorophosphate |
| DIPEA | N,N-diisopropylethylamine |
| DMAP | 4-dimethylaminopyridine |
| DMSO | dimethylsulfoxide |
| EDCI | 1-ethyl-3-(3-dimethylaminopropyl)carbodiimide |
| ESI | electrospray ionization |
| EtOH | ethanol |
| HRMS | high resolution mass spectrometry |
| HPLC | high pressure liquid chromatography |
| mL | milliliter |
| mg | milligram |
| mM | millimolar |
| m/z | mass-to-charge ratio |
| nm | nanometer |
| THTPA | thiamine triphosphatase |
| UV | ultraviolet |
| vol | volume |
| μL | microliter |

### Compounds

Provided herein are bi-functional linkers comprising two functional group wherein one of the functional group is a chemical group that is capable of generating highly reactive chemical species, such as carbene or nitrene or free-radical, that can be used to label a compound by utilizing the high reactivity of the carbene or nitrene or free-radical to create a collection of isomeric compounds that have spatial structural diversity. The other functional group of the linker is used to link with a labeling molecule, such as a specific oligonucleotide, and the synthesis of the isomeric assembly is encoded using the sequence of the oligonucleotide. Finally, a variety of compound can be labeled by such bi-functional linkers and the labeling molecule to produce labeled products, which can be mixed together to build a compound library. In the present disclosure, carbene or nitrene or free radical chemical reactions that are compatible with oligonucleotide labels are optimized, e.g. the solvents, the order of the reactions, the composition of the catalyst, and the like are optimized. The method avoids the complex structure-activity relationship experiment in the study of compound function, which is simple and fast in operation and high in screening efficiency.

Provided herein are methods for site non-selective labeling of organic chemicals using oligonucleotides, compounds useful in the methods, as well as the labeled compounds, libraries comprising the labeled compounds and uses thereof.

### Labeling Methods

The methods described herein utilize site non-selective reaction such as carbene or nitrene or free radical reactions and can simultaneously label an organic compound on different sites independent of the compound's functional groups, therefore minimize false negative results. The methods are also useful in labeling organic compounds that do not have functional groups. Further, the methods do not use reagents such as strong bases or strong reducing reagents to which the oligonucleotide tags are not stable.

In some embodiments, the method may be depicted as shown in Scheme 1:

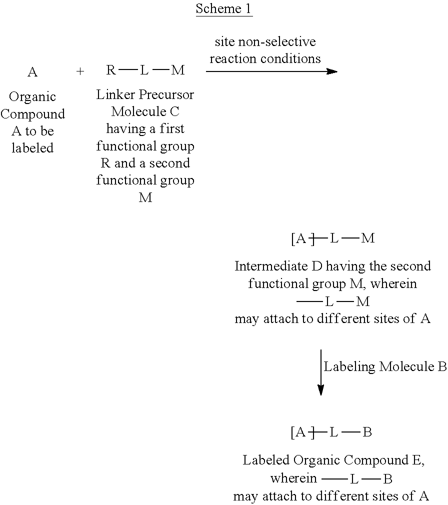

In some embodiments, provided is a method for labeling an organic compound A which method comprises:

(1) contacting a linker precursor molecule C with the organic compound A under site non-selective reaction conditions, e.g., carbene or nitrene or free-radical reaction conditions, wherein the linker precursor molecule C has two functional groups, a first functional group R and a second functional group M, wherein the first functional group R of the linker precursor molecule C generates a site non-selective reacting group, e.g., a carbene or nitrene or free-radical, under the site non-selective reaction conditions that reacts with the organic compound A, the second functional group is unreactive under the conditions when the first functional group reacts with the organic compound A, and wherein contacting of the linker precursor molecule C with the organic compound A forms an intermediate D having the second functional group M of the linker precursor molecule C. When two or more isomers are produced by the reaction of the linker precursor molecule C with the organic compound A under site non-selective reaction conditions, “intermediate D” as used herein may refer to all of the isomers, or one or some of the isomers.

In some embodiments, the method further comprises:

(2) contacting the intermediate D with a labeling molecule B thereby the second functional group M of the linker precursor molecule C reacts with the labeling molecule B to form the labeled organic compound E, wherein the labeling molecule B comprises a label. When two or more isomers are produced by the method, “labeled organic compound E” as used herein may refer to all of the isomers, or one or some of the isomers.

In some embodiments, the site non-selective reaction conditions are carbene reaction conditions, and the first functional group R of the linker precursor molecule C generates a carbene under the carbene reaction conditions. In some embodiments, the site non-selective reaction conditions are nitrene reaction conditions and the first functional group R of the linker precursor molecule C generates a nitrene under the nitrene reaction conditions. In some embodiments, the site non-selective reaction conditions are free radical reaction conditions and the first functional group R of the linker precursor molecule C generates a free-radical under the free radical reaction conditions.

The method provided herein labels organic compounds independent of any functions groups on the organic compounds. Accordingly, in some embodiments, the first functional group R of the linker precursor molecule C can react with multiple sites of the organic compound A to form a mixture of a plurality of isomeric products. In some embodiments, the organic compound A does not comprise a reactive functionality.

In some embodiments, the intermediate D is a mixture of a plurality of isomers.

In some embodiments, the labeled organic compound E is a mixture of a plurality of isomers.

In some embodiments, the label comprises a unique sequence for chemical labeling or a fluorescent tag (e.g., a fluorophore or GFP) or biotin label, or a combination thereof. In some embodiments, the unique sequence comprises single-strand DNA or RNA, double-strand DNA or RNA, multi-strand DNA or RNA, or other chemically modified oligonucleotide. In some embodiments, the label comprises an oligonucleotide.

In some embodiments, the unique sequence comprises a chemically modified oligonucleotide including natural nucleotide or any other artificial nucleotide or the mixture of both. In some embodiments, the chemically modified oligonucleotide is modified through an amino group on the oligonucleotide. In some embodiments, the chemically modified oligonucleotide comprises an acetylenyl (C≡CH) group. In some embodiments, the acetylenyl group is attached to the oligonucleotide through a linker. In some embodiments, the linker comprises —CH₂—(OCH₂CH₂)ₙ—O—CH₂—, wherein n is an integer from 0 to 10, or from 1 to 5.

In some embodiments, the site non-selective reaction conditions, such as carbene or nitrene or free-radical reaction conditions, comprise dissolving the organic compound A and the linker precursor molecule C in a solvent to form a reaction mixture. In some embodiments, the free-radical reaction conditions comprise UV (e.g., 365 nm) radiation of the reaction mixture for a period of time, such as 1-5 hours or about 3 hours or longer, at ambient temperature. In some embodiments, the site non-selective reaction conditions, such as carbene or nitrene or free-radical reaction conditions, comprise a radical initiator, such as a peroxide (a compound having a peroxide bond (—O—), e.g., di-tert-butyl peroxide, benzoyl peroxide, methyl ethyl ketone peroxide, and peroxydisulfate) or a transition metal complex. Other radical initiators are generally known in the art, see, e.g., Denisov, et al., Handbook of Free Radical Initiators, Wiley-Interscience; 1st edition (Apr. 4, 2003), which is hereby incorporated by reference in its entirety.

In some embodiments, the method further comprises isolating the intermediate D of step (1) and/or the labeled organic compound E of step (2) above. Isolating means separating the desired product(s) of a reaction from other materials in the reaction mixture, such as solvent, reagents, byproducts, etc. If the method produces a plurality of isomeric and/or disproportionation reaction products, such products may be isolated as a mixture or as an individual compound.

### Linker Precursor Molecules

The methods described herein use a linker precursor molecule to connect an organic molecule being labeled with a labeling molecule through the linker of the linker precursor molecule. The linker precursor molecules have functional groups that are capable of generating carbene or nitrene or free radicles which can react with an organic compound independent of any functional groups of the organic compound.

In some embodiments, provided is a linker precursor molecule C comprising a first functional group and a second functional group, wherein the first functional group is capable of generating a site non-selective reacting group, such as a carbene or nitrene or free-radical, that is capable of reacting with an organic compound A, and the second functional group is unreactive with the organic compound A under the conditions when the first functional group reacts with the organic compound A, but is capable of reacting with a labeling molecule B.

In some embodiments, the linker precursor molecule C is of the formula R-L-M, wherein R is the first functional group, M is the second functional group and L is a linker moiety, wherein L comprises one or more of moieties independently selected from optionally substituted alkylene, alkenylene, alkynylene, heteroalkylene, cycloalkylene, heterocycloalkylene, arylene, and heteroarylene.

In some embodiments, L is an alkylene wherein one or more of the methylene units of the alkylene is optionally replaced with a moiety independently selected from the group consisting of NR¹, O, S, SO, SO₂, CO, NR¹C(O), C(O)NR¹, cycloalkylene, heterocycloalkylene, arylene, and heteroarylene, wherein each R¹ is independently H or alkyl.

In some embodiments, L is an alkylene wherein one or more of the methylene units of the alkylene is replaced with a moiety independently selected from phenylene, O, NHC(O) and C(O)NH.

In some embodiments, L comprises a phenylene.

In some embodiments, the first functional group or R of the linker precursor molecule C comprises a group that is capable of generating a carbene or nitrene group or free radical (such as a diazo, ketene, isocyanate, or azide group). In some embodiments, the first functional group or R of the linker precursor molecule C comprises a diazirine, aryl azide, benzophenone, or diazo.

In some embodiments, the first functional group or R comprises an arylene group and is attached to the rest of the linker precursor molecule C through the arylene group, such as a phenylene group.

In some embodiments, the first functional group or R of the linker precursor molecule C is selected from the group consisting of

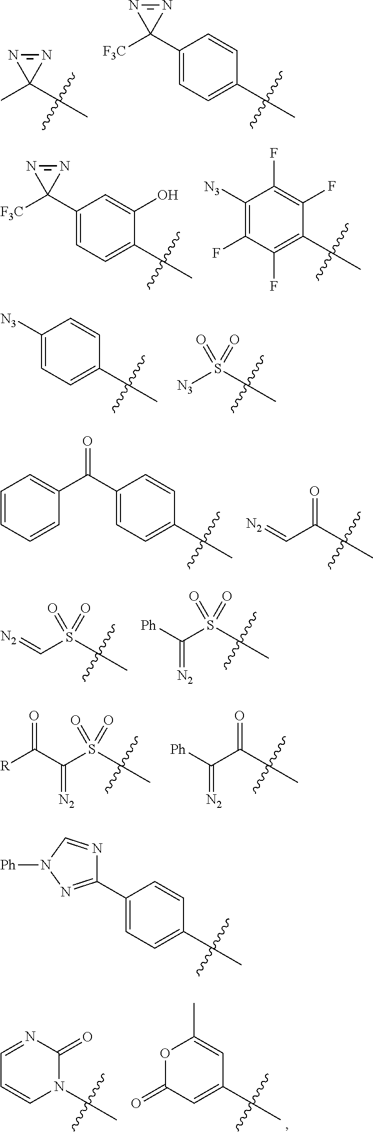

wherein [symbol] represents the point of attachment to the rest of the linker precursor molecule C.

In some embodiments, the second functional group or M of the linker precursor molecule C comprises an azide (e.g., an alkylazide), alkyne, ene, or —C(O)—O—.

In some embodiments, the second functional group or M comprises an alkylene group and is attached to the rest of the linker precursor molecule C through the alkylene group.

In some embodiments, the second functional group or M of the linker precursor molecule C is selected from the group consisting of

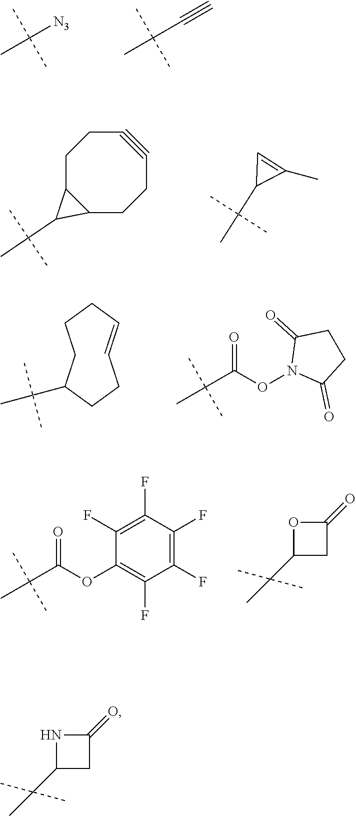

wherein [symbol] represents the point of attachment to the rest of the linker precursor molecule C.

In some embodiments, the linker precursor molecule C is selected from the group consisting of

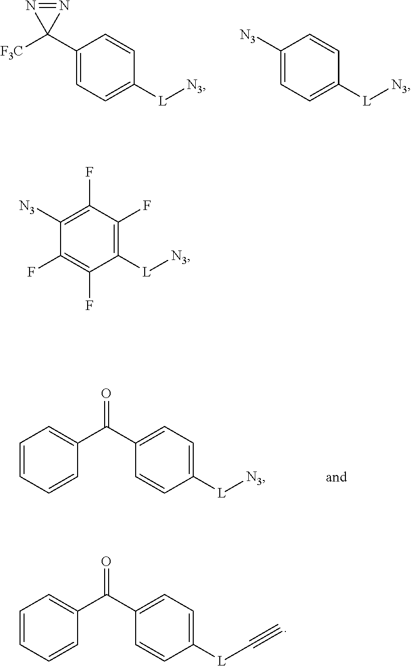

In some embodiments, the linker precursor molecule C is selected from the group consisting of

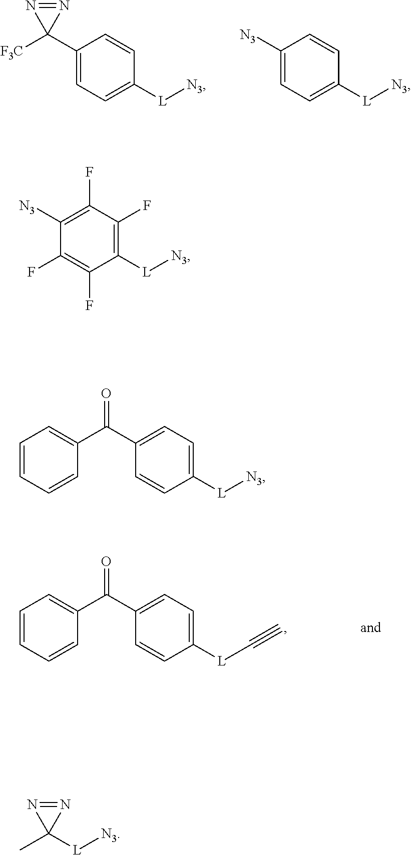

In some embodiments, the linker precursor molecule C is selected from the group consisting of

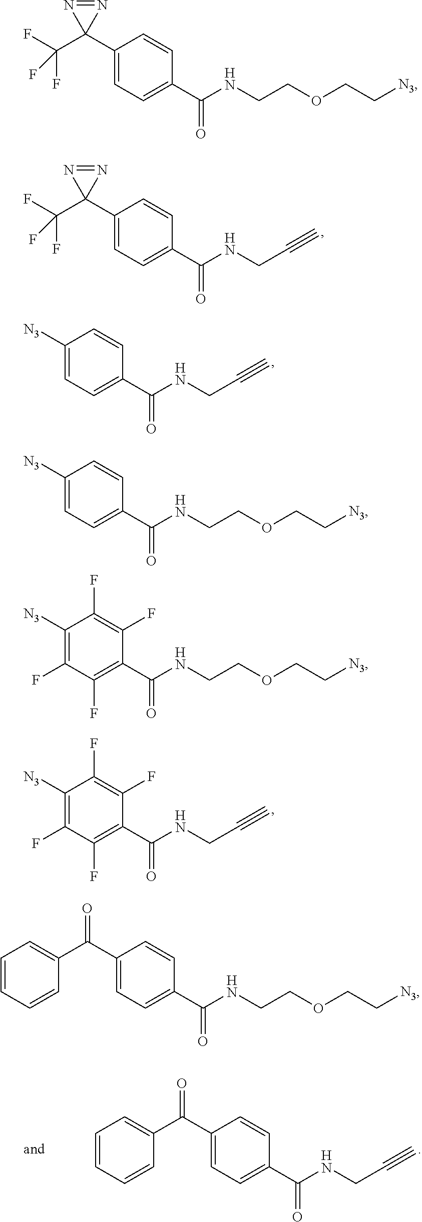

In some embodiments, the linker precursor molecule C is selected from the group consisting of

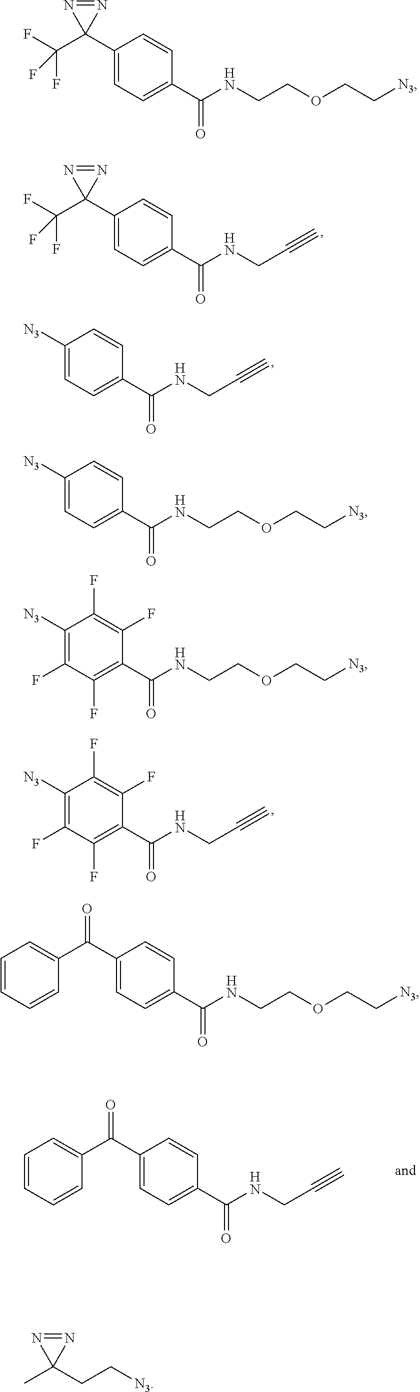

In some embodiments, the linker precursor molecule C is

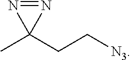

### Labeled Organic Compounds

In some embodiments, provided is a labeled organic compound E which is produced by a method comprising:

(1) contacting a linker precursor molecule C with an organic compound A under site non-selective reaction conditions, such as carbene or nitrene or free-radical reaction conditions, wherein the linker precursor molecule C has two functional groups, a first functional group R and a second functional group M, wherein the first functional group R of the linker precursor molecule C generates a site non-selective reacting group, such as carbene or nitrene or free-radical, that reacts with the organic compound A, under the site non-selective reaction conditions, the second functional group is unreactive under the conditions when the first functional group reacts with the organic compound A, and wherein contacting of the linker precursor molecule C with the organic compound A forms an intermediate D having the second functional group M of the linker precursor molecule C; and

(2) contacting the intermediate D with a labeling molecule B thereby the second functional group M of the linker precursor molecule C reacts with the labeling molecule B to form the labeled organic compound E, wherein the labeling molecule B comprises a label.

In some embodiments, the organic compound A does not comprise a reactive functionality.

In some embodiments, the intermediate D is a mixture of a plurality of isomers.

In some embodiments, the labeled organic compound E is a mixture of a plurality of isomers.

In some embodiments, the label comprises a unique sequence for chemical labeling, or a fluorescent tag (e.g., a fluorophore or GFP) or biotin label, or a combination thereof.

In some embodiments, the unique sequence comprises single-strand DNA or RNA, double-strand DNA or RNA, a chemically modified oligonucleotide, a multi-strand natural or artificial oligonucleotide or an artificial oligonucleotide.

In some embodiments, the unique sequence comprises an oligonucleotide.

In some embodiments, provided is a set of isomeric labeled organic compounds comprising a label moiety and an organic compound moiety wherein the label moiety is attached to different sites of the organic compound moiety through a linker.

In some embodiments, provided is a mixture comprising (1) a plurality of isomeric compounds wherein the isomeric compounds are produced by a site non-selective reaction, such as a carbene, nitrene, or free radical reaction of an organic compound A with a linker precursor molecule C described herein, and/or (2) one or more pairs of compounds that are products of a disproportionation reaction of an isomeric compound produced by the free radical reaction of (1).

In some embodiments, the pair of disproportionation reaction products comprise a compound F having a molecular weight that is the molecular weight of the isomeric compound E produced by the site non-selective reaction plus 2, and a compound F′ having a molecular weight that is the molecular weight of the isomeric compound E produced by the site non-selective reaction minus 2.

In some embodiments, provided is a mixture of a plurality of isomeric labeled compounds which is produced by a method comprising:

(1) reacting the first functional group of the linker precursor molecule C having a first and a second functional groups as described herein with an organic compound A under site non-selective reaction conditions, such as carbene or nitrene or free-radical reaction conditions, to form an intermediate D having the second functional group of the linker precursor molecule C; and

(2) reacting the second functional group with a labeling molecule B comprising a label.

In some embodiments, the label comprises a unique sequence for chemical labeling, or a fluorescent tag (e.g., a fluorophore or GFP) or biotin label, or a combination thereof.

In some embodiments, the unique sequence comprises single-strand DNA or RNA, double-strand DNA or RNA, other chemically modified oligonucleotide, or other artificial oligonucleotide, such as a multi-strand natural or artificial oligonucleotide. In some embodiments, the unique sequence comprises an oligonucleotide.

In some embodiments, the first functional group of the linker precursor molecule C reacts with multiple sites of the organic compound A to form a mixture of a plurality of isomeric intermediates D, which react with the labeling molecule B to form a plurality of isomeric labeled organic compounds E. In some embodiments, provided is a library of labeled organic compounds comprising at least two labeled organic compounds (or two sets of isomeric labeled organic compounds) each produced according to a method described herein, wherein a unique organic compound is labeled with a unique label.

In some embodiments, the library comprises at least 5, 10, 20, 30, 40, 50, 60, 70, 80, 90, 100, 200, 300, 400, or 500 unique labeled organic compounds (or 5, 10, 20, 30, 40, 50, 60, 70, 80, 90, 100, 200, 300, 400, or 500 sets of isomeric labeled organic compounds) each labeled with a unique label.

In some embodiments, the unique label is an oligonucleotide having a unique sequence.

### Screening Methods

In some embodiments, provided is a method of identifying organic compounds that bind to a target molecule, comprising assaying a labeled organic compound, a set of isomeric labeled organic compounds, or a library of labeled organic compounds described herein, and identifying the organic compounds that bind to the target according to their labels. Such assays are generally known in the art, see, e.g., Chapter, 13 and 16, in A HANDBOOK FOR DNA-ENCODED CHEMISTRY, Goodnow Jr., Wiley, 1 edition (2014) and Decurtins, W., et al., Automated screening for small organic ligands using DNA-encoded chemical libraries. Nat Protoc. 2016; 11(4): 764-80, which hereby are incorporated by reference in their entireties.

In some embodiments, the organic compound is screened by the affinity between the labeled organic molecule described herein and the target molecule to be tested. Targets to be tested include protein molecules, cells, cellular organelles (nucleus, mitochondria, golgi, peroxisome, lysosome, exosome etc), cytoskeleton (microtubules, intermediate filament, microfilaments), DNA, RNA, sugars, phospholipid molecules, phospholipid protein complexes, or the complex of the mentioned above and the like. Such screening methods are generally known in the art, and may vary depending on the specific use, such as the particular target to be tested.

In some embodiments, the labeled organic compounds that have binding affinity with the target molecule are selected for identification. In some embodiments, the selected labeled organic compounds are subjected to a sequencing reaction of its label to interpret the encoded oligonucleotide sequences (for example, Sanger sequencing, second-generation sequencing, etc.) to identify the organic compounds that bind to the target.

In some embodiments, provided is a method for identifying a compound having two conjugated double bonds, the method comprises

(1) reacting the first functional group of the linker precursor molecule C described herein with an organic compound A under site non-selective reaction conditions, such as carbene or nitrene or free-radical reaction conditions, to form a product or a mixture of products D, wherein the second functional group of the linker precursor molecule C remains unreacted; and

(2) analyzing the product or mixture of products D, wherein the presence of a pair of disproportionation reaction products indicates that the organic compound A has two conjugated double bonds, wherein the pair of disproportionation reaction products comprise a compound F having a molecular weight that is the molecular weight of a product D of the site non-selective reaction plus 2, and a compound F′ having a molecular weight that is the molecular weight of a product D of the site non-selective reaction minus 2.

In some embodiments, at least one of the two conjugated double bonds is in a ring system, such as a cycloalkyl ring.

The product or mixture of products may be analyzed by methods generally known in the art, such as liquid chromatography in combination with mass spectrometry.

### Methods of Treatment

In some embodiments, provided herein is a compound for use in the treatment of a disease or condition in a subject in need thereof, wherein the disease or condition is modulated or affected by the activity of a predetermined biological target. In some embodiments, the compound is identified by the screening methods described herein, and optionally further modified, e.g., to enhance binding efficacy to the predetermined biological target. In certain embodiments, the compound is derived from a labeled organic compound as described herein. The predetermined biological target can be any, including but not limited to, proteins, enzymes, cells, cellular organelles (nucleus, mitochondria, golgi, peroxisome, lysosome, exosome etc), cytoskeleton (microtubules, intermediate filament, microfilaments), DNA, RNA, sugars, phospholipid molecules, phospholipid protein complexes, or the complex of the mentioned above, and the like.

In certain embodiments, the predetermined biological target is a poly (ADP-ribose) polymerase (PARP). Poly (ADP-ribose) polymerase (PARP) is a family of proteins involved in a number of cellular processes such as DNA repair, genomic stability, and programmed cell death. The PARP family comprises 17 members, such as PARP1, PARP2, VPARP (PARP4), Tankyrase-1 and -2 (PARP-5a or TNKS, and PARP-5b or TNKS2), PARP3, PARP6, TIPARP (or “PARP7”), PARP8, PARP9, PARP10, PARP11, PARP12, PARP14, PARP15, and PARP16.

In certain embodiments, the predetermined biological target is PARP1. PARP1 is a protein that is important for repairing single-strand breaks. Drugs that inhibit PARP1 cause multiple double strand breaks to form, and in tumors with BRCA1, BRCA2 or PALB2 mutations, these double strand breaks cannot be efficiently repaired, leading to the death of the tumor cells. Some cancer cells that lack the tumor suppressor PTEN may be sensitive to PARP inhibitors because of downregulation of Rad51. Hence PARP inhibitors can be effective against PTEN-defective tumors.

In addition to their use in cancer therapy, PARP inhibitors are considered a potential treatment for stroke, myocardial infarction, and neurodegenerative diseases.

Accordingly, in some embodiments, provided is a method for treating a cancer, stroke, myocardial infarction, a neurodegenerative disease or inflammation in a subject in need thereof, comprising administering to the subject a therapeutically effecting amount of a compound identified as capable of binding or inhibiting PARP1 by the screening methods described herein.

In some embodiments, provided is a method for treating a cancer, stroke, myocardial infarction, a neurodegenerative disease or inflammation in a subject in need thereof, comprising administering to the subject a therapeutically effecting amount of luteolin, naringin, hyeroside, liquiritin, epicatechin, epigallocatechin, daphnetin, F001, F002, F003 or F006, or a derivative thereof. In some embodiments, provided is a method for treating a cancer, stroke, myocardial infarction, a neurodegenerative disease or inflammation in a subject in need thereof, comprising administering to the subject a therapeutically effecting amount of luteolin, or a derivative thereof.

In some embodiments, the subject in need of treatment has cancer. In some embodiments, the subject has a cancer cell with a BRCA1, BRCA2, PALB2 or PTENT mutation. In certain embodiments, the mutation is an inactivating mutation.

In some embodiments, the subject has a cancer cell that overexpresses a PARP protein as compared to counterpart normal cell. In some embodiments, the PARP protein is PARP1.

In some embodiments, the subject in need of treatment has a neurodegenerative disease. In some embodiments, the neurodegenerative disease is selected from the group consisting of Parkinson's disease, Alzheimer's disease, Huntington's disease, atrophic myelitis, AIDS dementia, vascular dementia and combinations thereof.

In some embodiments, the subject in need of treatment has suffered a stroke.

In some embodiments, the subject in need of treatment sufferes from inflammation, such as, but not limited to, inflammation associated with a disease or condition selected from the group consisting of Parkinson's disease, arthritis, rheumatoid arthritis, multiple sclerosis, psoriasis, psoriatic arthritis, Crohn's disease, inflammatory bowel disease, ulcerative colitis, lupus, systemic lupus erythematous, juvenile rheumatoid arthritis, juvenile idiopathic arthritis, Grave's disease, Hashimoto's thyroiditis, Addison's disease, celiac disease, dermatomyositis, multiple sclerosis, myasthenia gravis, pernicious anemia, Sjogren syndrome, type I diabetes, vasculitis, uveitis, atherosclerosis and ankylosing spondylitis.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of a predetermined biological target, such as, but not limited to, a PARP protein. The therapeutically effective amount may vary depending on the subject, and disease or condition being treated, the weight and age of the subject, the severity of the disease or condition, and the manner of administering, which can readily be determined by one or ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

Improvements in any of the foregoing response criteria are specifically provided by the methods of the present disclosure.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1

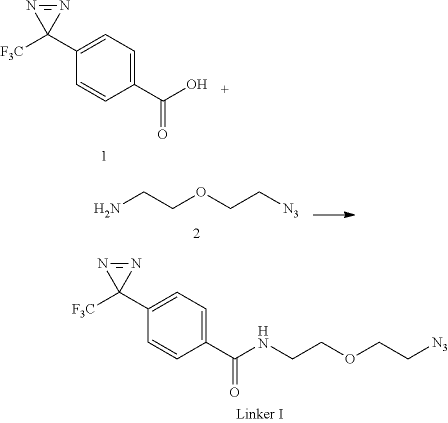 [SMILES: O=C(O)c1ccc(C2(C(F)(F)F)N=N2)cc1 ; [N-]=[N+]=NCCOCCN ; [N-]=[N+]=NCCOCCNC(=O)c1ccc(C2(C(F)(F)F)N=N2)cc1]

Compound 1 (200 mg) was dissolved in 10 mL dichloromethane and then mixed with compound 2 (200 mg), EDCI (416 mg) and DMAP (10 mg) with stirring. After stirring at room temperature for 3 hours, the mixture was extracted with dichloromethane, washed with brine, concentrated and purified by flash chromatography to generate linker I (210 mg, 70.4%). NMR (500 MHz, Chloroform-d) δ 7.82 (d, J=8.5 Hz, 2H), 7.25 (d, J=8.2 Hz, 2H), 6.63 (s, 1H), 3.79-3.62 (m, 6H), 3.45-3.36 (m, 2H); HRMS-ESI (m/z) [M+H]⁺ calculated for C₁₃H₉F₃N₆O₂, 343.1130, found, 343.1133.

### Example 2

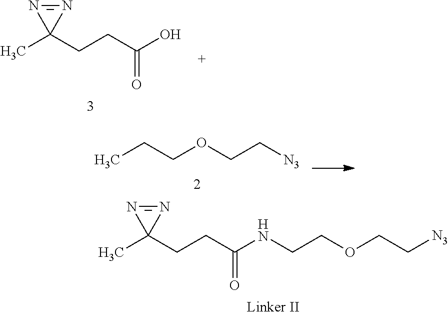

Compound 3 (200 mg) was dissolved in dichloromethane 10 mL, and then mixed with compound 2 (513 mg), EDCI (405 mg) and DMAP (10 mg) with stirring. After stirring at room temperature for 3 hours, the mixture was extracted with dichloromethane, washed with brine, concentrated and purified by flash chromatography to generate linker II (283 mg, 75.6%). ¹H NMR (500 MHz, Chloroform-d) δ 5.93 (s, 1H), 3.72-3.66 (m, 2H), 3.57 (t, J=5.0 Hz, 2H), 3.48 (t, J=5.1 Hz, 2H), 3.41-3.35 (m, 2H), 2.06-1.98 (m, 2H), 1.82-1.67 (m, 2H), 1.03 (s, 3H); HRMS-ESI (m/z) [M+H]⁺ calculated for C₉H₁₇N₆O₂, 241.1413, found, 241.1417.

### Example 3

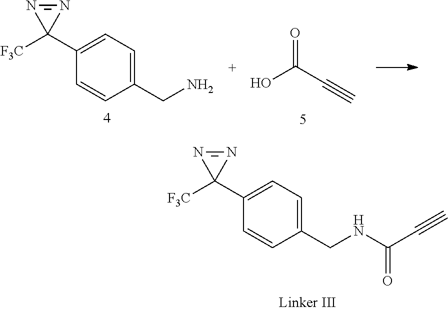

Compound 4 (50 mg) was dissolved in 5 mL dichloromethane 5 mL, and then mixed with compound 5 (25 μL), HATU (150 mg) and DIPEA (1384), after stirring at room temperature for 3 hours, the mixture was extracted with dichloromethane, washed with brine, concentrated and purified by flash chromatography to give linker III (49 mg, 79.1%). NMR (500 MHz, Chloroform-d) δ 7.81 (d, J=8.5 Hz, 2H), 7.26 (d, J=8.5 Hz, 2H), 6.23 (s, 1H), 4.26 (dd, J=5.2, 2.6 Hz, 2H), 2.30 (t, J=2.6 Hz, 1H). HRMS-ESI (m/z) [M+H]⁺ calculated for C₁₂H₉F₃N₃O, 268.0698, found, 268.0699.

### Example 4

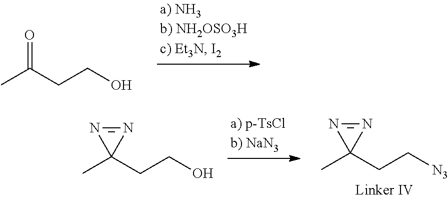

3-(2-azidoethyl)-3-methyl-3H-diazirine (linker IV) was prepared according to Liang, et al. (Angew Chem Int Ed Engl (2017) 56 (10):2744-2748) by switching the solvent N,N-dimethylformamide with acetonitrile. ¹H NMR (500 MHz, Chloroform-d)=1.05 (s, 3H), 1.60 (t, 2H, J=5.4 Hz), 3.18 (t, 2H, J=5.4 Hz).

### Example 5

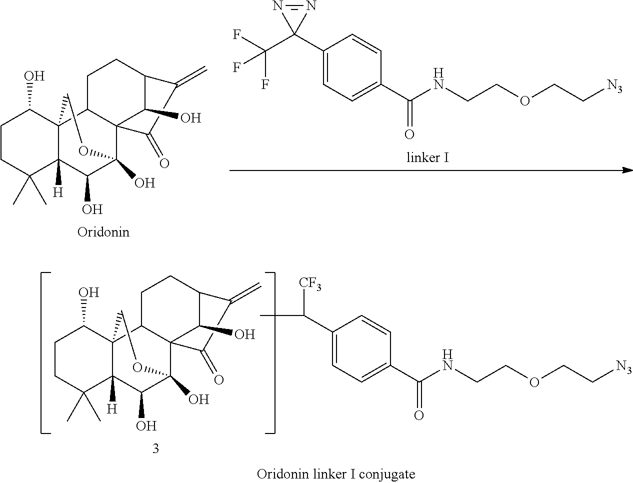

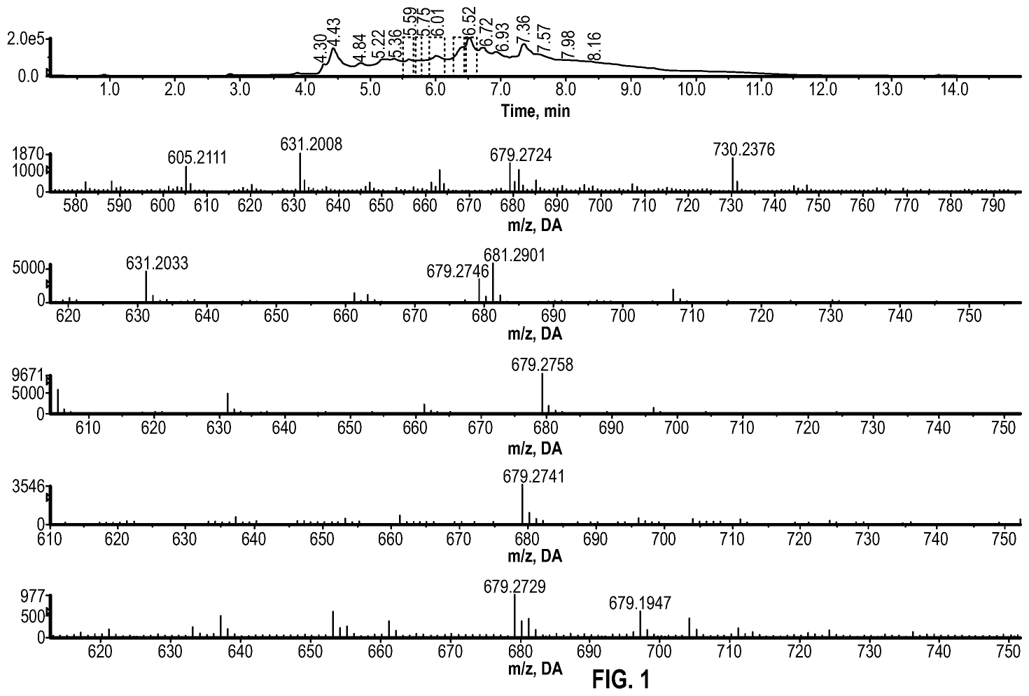

Oridonin (2 mg, 0.0054 mM) was dissolved in 1 mL dichloromethane and mixed with bifunctional linker I (3.3 mg, 0.011 mM). The mixture was irradiated with UV light (365 nm) for 3 hours at room temperature to generate oridonin linker I conjugates, which comprise at least five isomeric products having MS (ESI (m/z) [M+H]⁺) of 679.27 (FIG. 1).

### Example 6

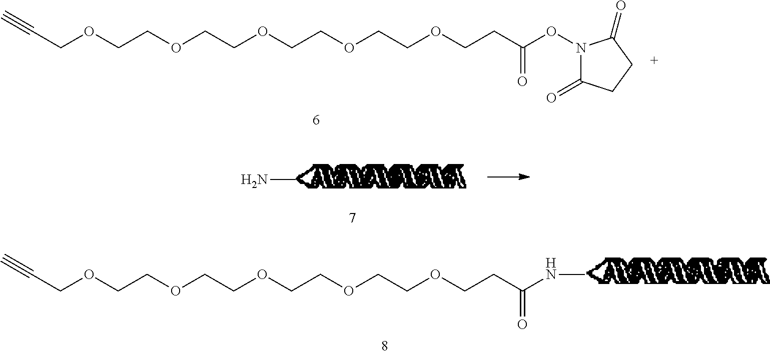

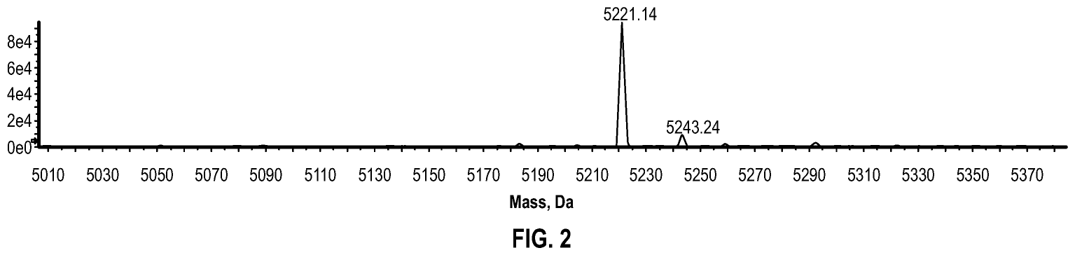

Initial DNA 7 (50 μL, 1 mM in borate buffer (pH 9.4)) was mixed with compound 6 (5 μL, 200 mM in DMSO) via vortex in a 500 μL, tube, and then rotated with a rotator for 10 hours. To the mixture was added 5 M NaCl (5.5 μL) followed by EtOH (160 μL). The contents were briefly vortex and incubated in freezer for 20 minutes at −20° C. The suspension was then centrifuged at 10,000×g for 5 minutes and the supernatant was discarded and trace of EtOH was removed under vacuum. The pellet was dissolved in H₂O (50 μL) to make the 1 mM solution of compound 8. (FIG. 2).

### Example 7

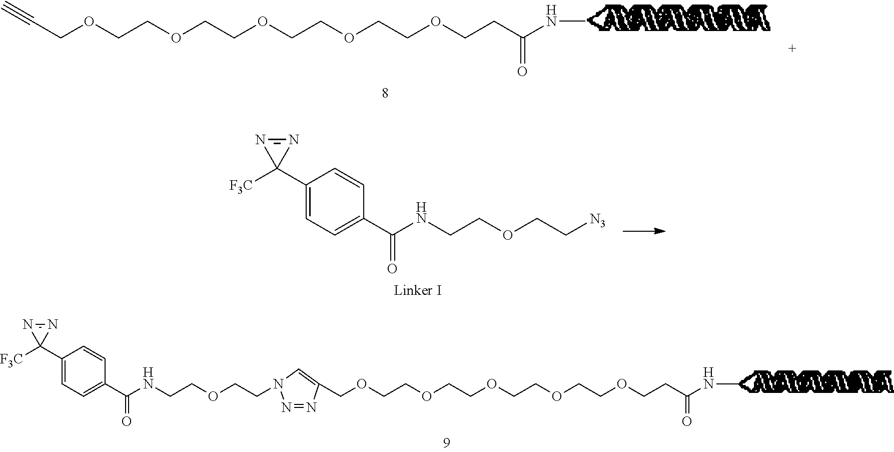 [SMILES: C#CCOCCOCCOCCOCCOCCC(=O)NC ; [N-]=[N+]=NCCOCCNC(=O)c1ccc(C2(C(F)(F)F)N=N2)cc1 ; CNC(=O)CCOCCOCCOCCOCCOCc1cn(CCOCCNC(=O)c2ccc(C3(C(F)(F)F)N=N3)cc2)nn1]

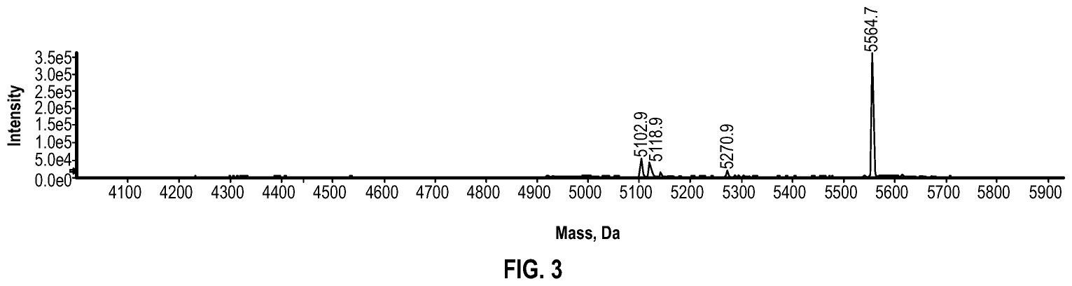

Compound 8 (20 μL, 1 mM in H₂O) was mixed with linker I (4 μL, 100 mM), copper(II) sulfate pentahydrate (4 μL, 100 mM), sodium ascorbate (4 μL, 200 mM) and THTPA (4 μL, 100 mM). The mixture was mixed by vortex followed by rotating on a rotator for 5 hours. Then to the mixture was added 5 M NaCl (3.6 μL) followed by EtOH (100 μL). The contents were briefly vortexed and incubated for 20 minutes at −20° C. The suspension was then centrifuged at 10,000×g for 5 minutes and the supernatant was discarded and trace of EtOH was removed under vacuum to generate labeled compound 9 (FIG. 3).

### Example 8

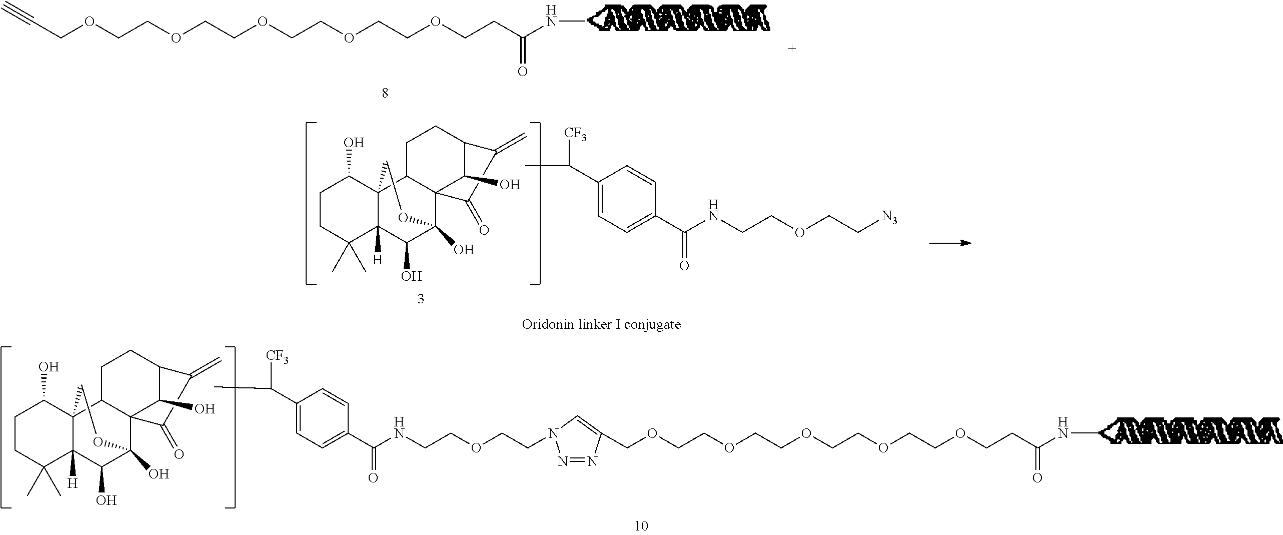

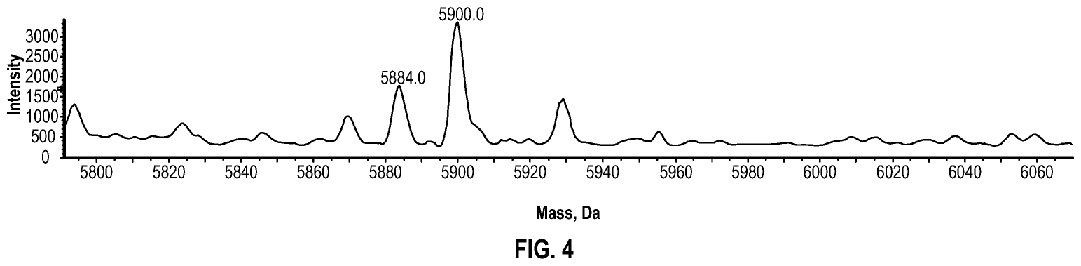

Compound 8 (20 μL, 1 mM in H₂O) was mixed with Oridonin-linker I conjugate (4 μL, 100 mM), copper(II) sulfate pentahydrate (4 μL, 100 mM), sodium ascorbate (4 μL, 200 mM) and THTPA (4 μL, 100 mM). The mixture was mixed by vortex and rotating on a rotator for 5 hours. Then to the mixture was added 5 M NaCl (3.6 μL) followed by EtOH (100 μL). The mixture were briefly vortexed and allowed to incubate for 20 minutes at −20° C. The suspension was then centrifuged at 10,000×g for 5 minutes and the supernatant was discarded and trace of EtOH was removed under vacuum to generate labeled compound 10 (FIG. 4).

### Example 9

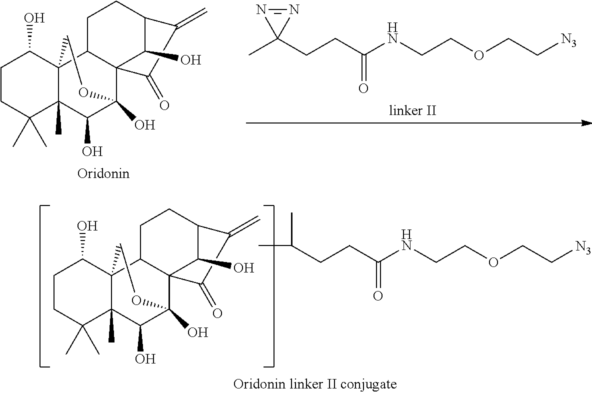

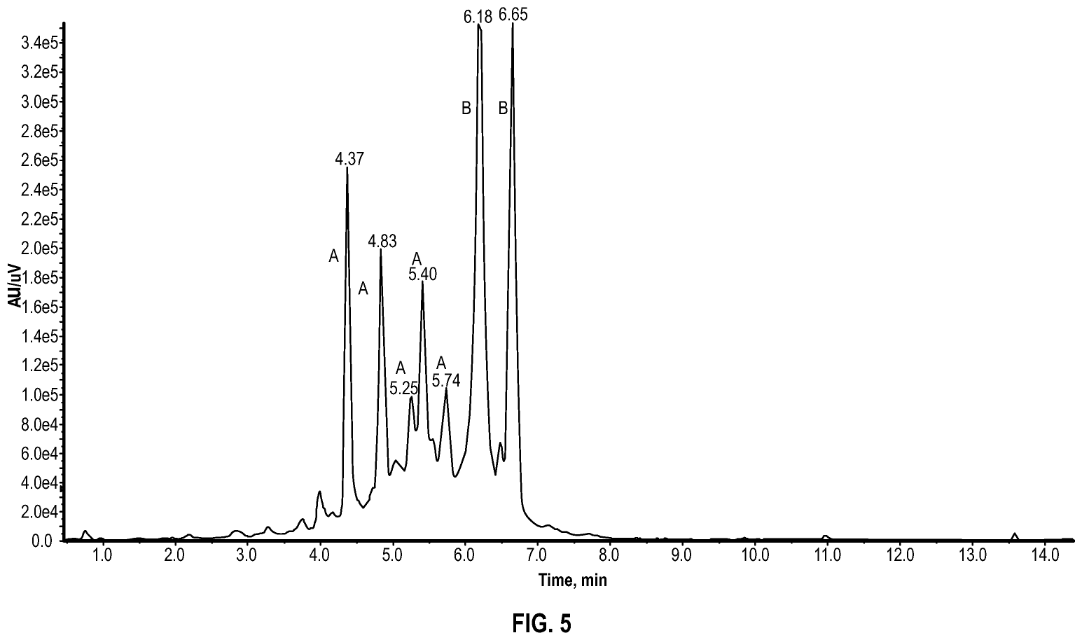

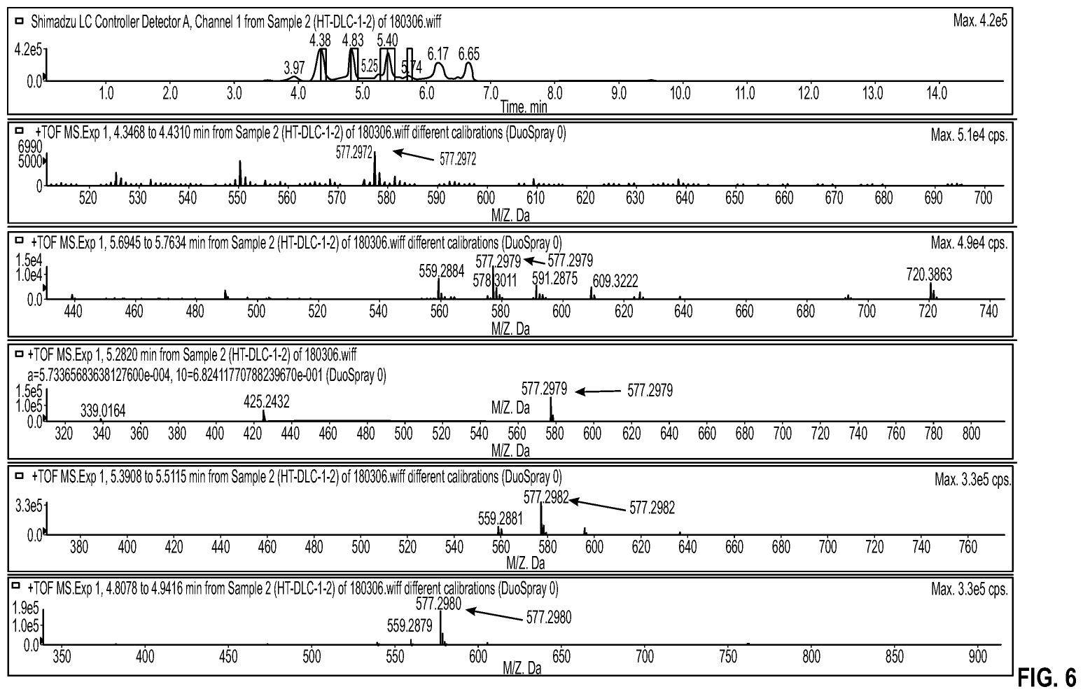

Oridonin (2 mg, 0.0054 mM) was dissolved in acetonitrile 1 mL, then mixed with bifunctional linker II (2.6 mg, 0.011 mM). The mixture was irradiated by UV light (365 nm) for 3 hours at room temperature to generate oridonin-linker II conjugate compounds, which comprise at least five isomeric compounds having MS (ESI (m/z) [M+H]⁺) of 577.3 (FIG. 5 and FIG. 6).

### Example 10

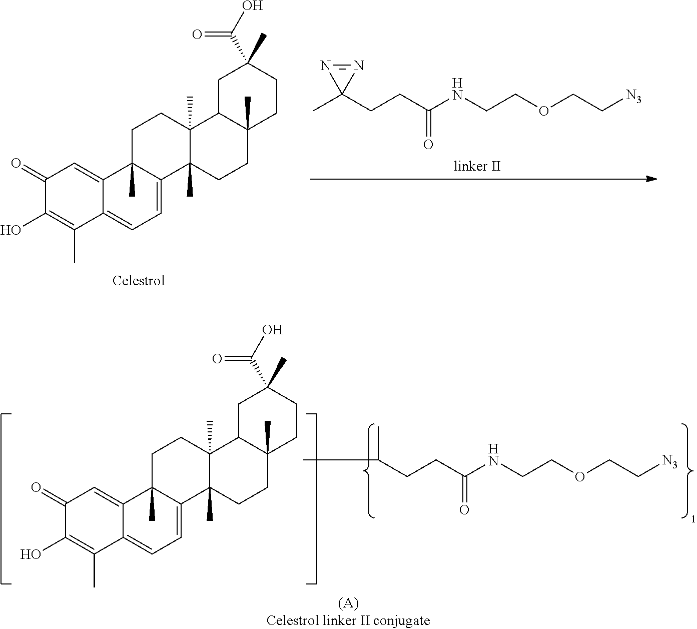

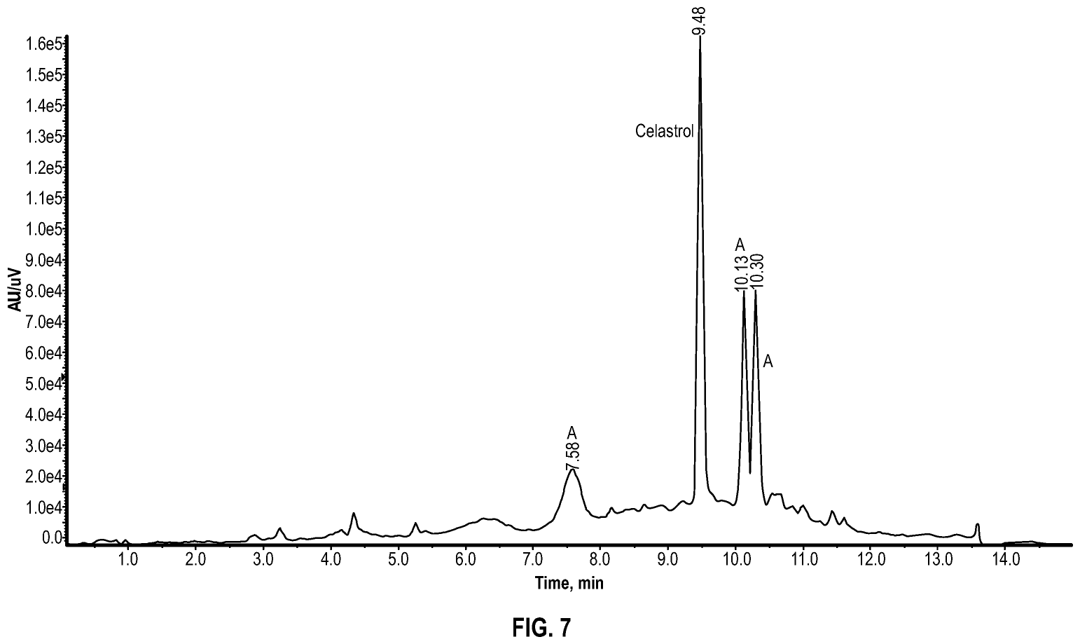

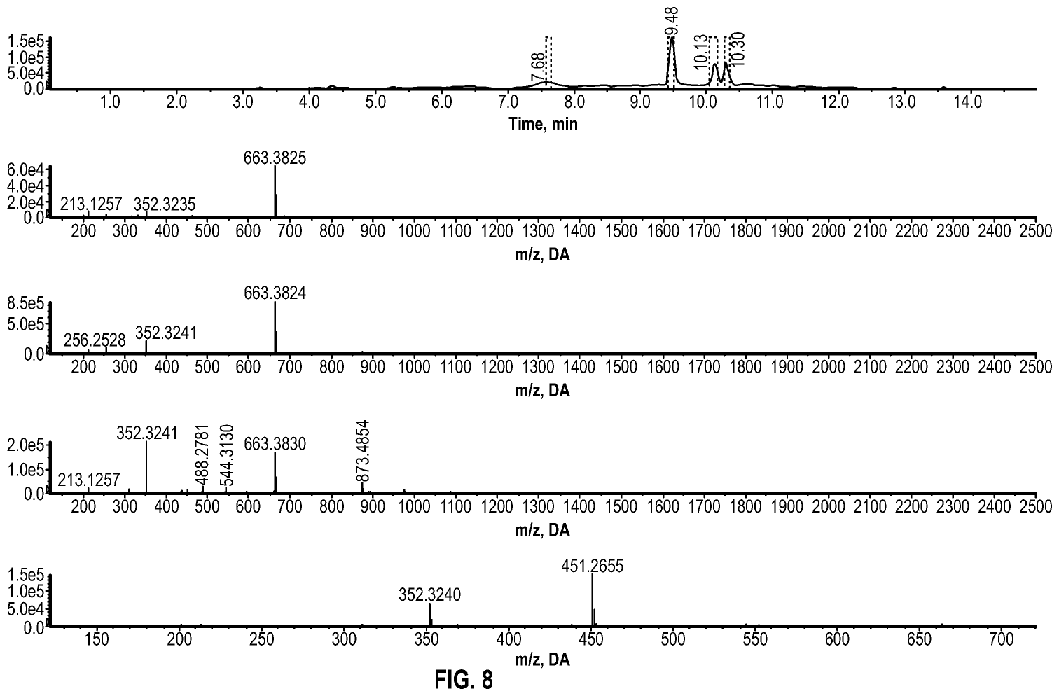

Celestrol (2 mg, 0.0044 mM) was dissolved in 1 mL acetonitrile, and then mixed with bifunctional linker II (2.1 mg, 0.009 mM). The mixture was irradiated by UV light (365 nm) for 3 hours at room temperature to generate celestrol-linker II conjugates, which comprise at least three isomeric compounds having MS (ESI (m/z) [M+H]⁺) of 663.38 (FIG. 7 and FIG. 8).

### Example 11

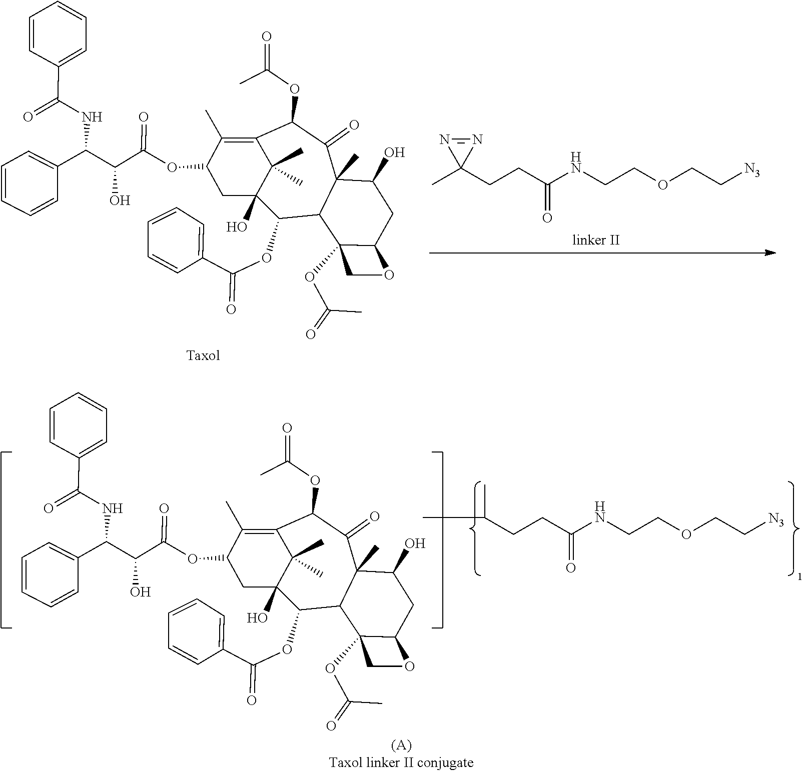

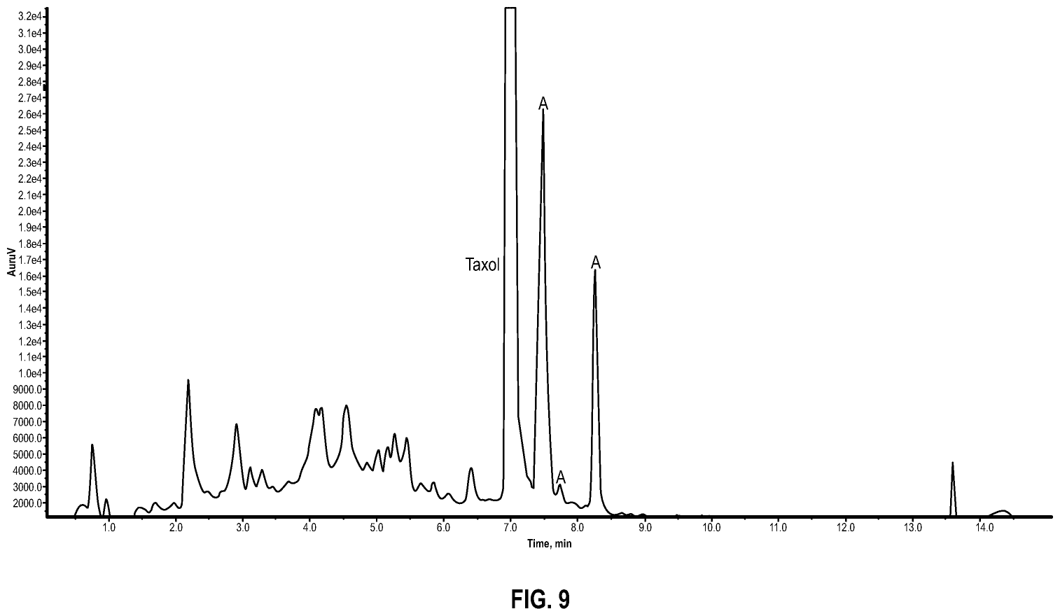

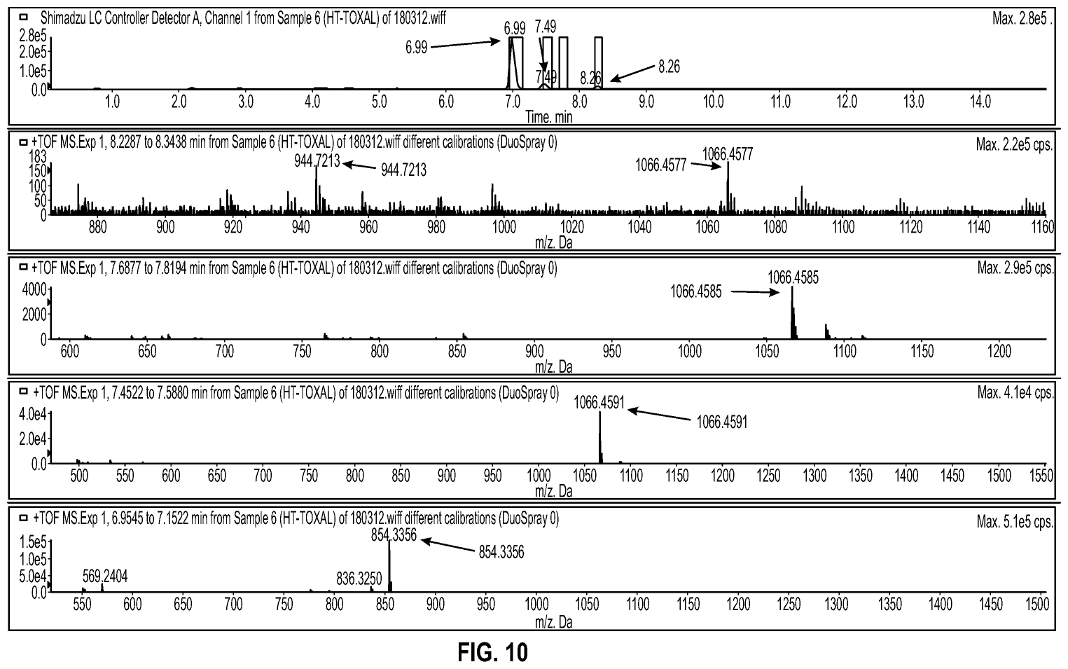

Taxol (2 mg, 0.0047 mM) was dissolved in acetonitrile 1 mL, and then mixed with bifunctional linker II (2.3 mg, 0.0094 mM). The mixture was irradiated by UV light (365 nm) for 3 hours at room temperature to generate taxol-linker II conjugates, which comprise at least three isomeric compounds having MS (ESI (m/z) [M+H]⁺) of 1066.46 (FIG. 9 and FIG. 10).

### Example 12

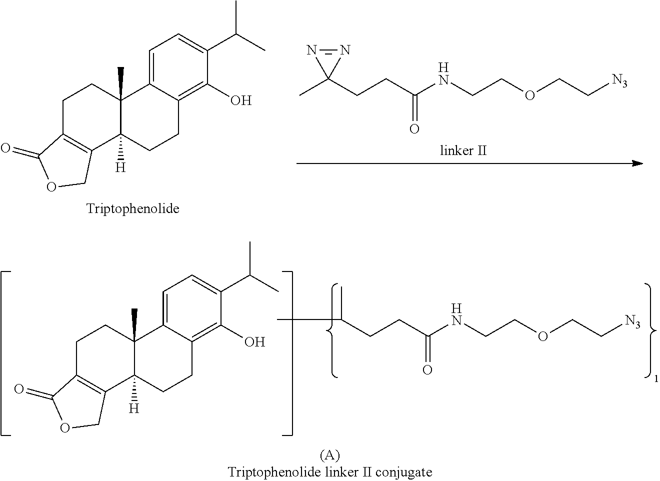

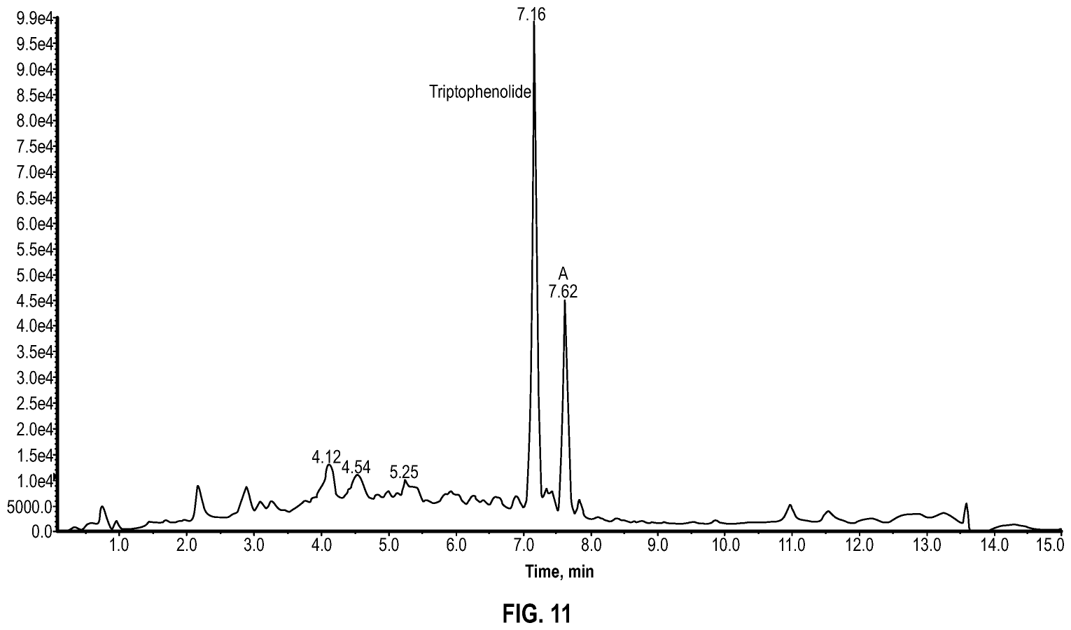

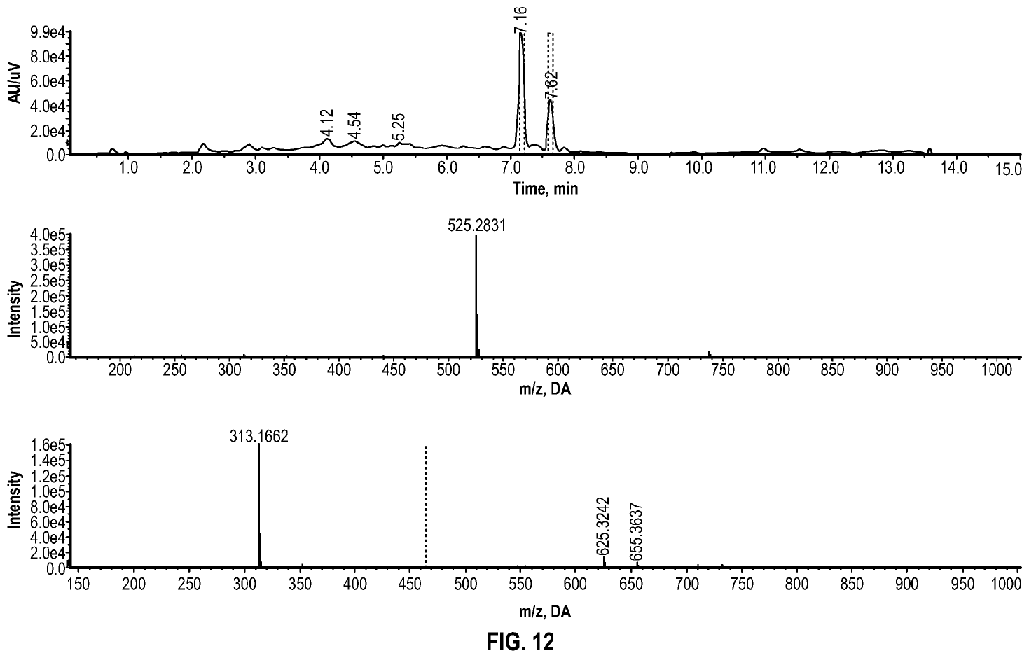

Triptophenolide (2 mg, 0.0064 mM) was dissolved in 1 mL acetonitrile, and then mixed with bifunctional linker II (3.1 mg, 0.0128 mM). The mixture was irradiated using UV light (365 nm) for 3 hours at room temperature to generate triptophenolide linker II conjugates (FIG. 11 and FIG. 12).

### Example 13

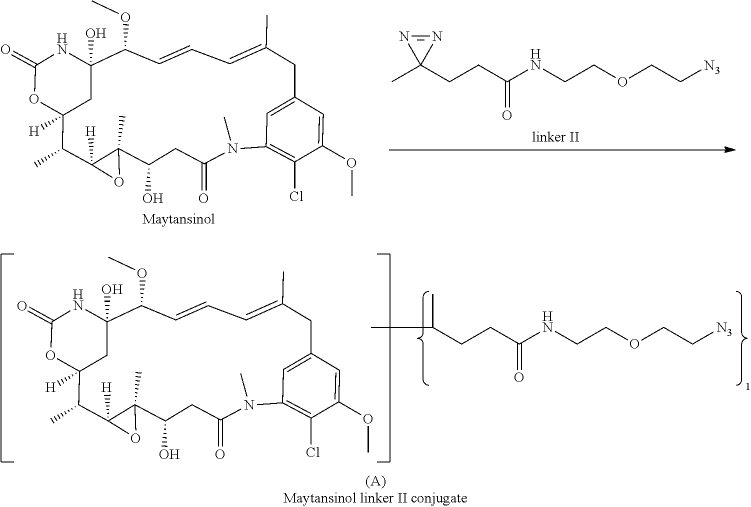

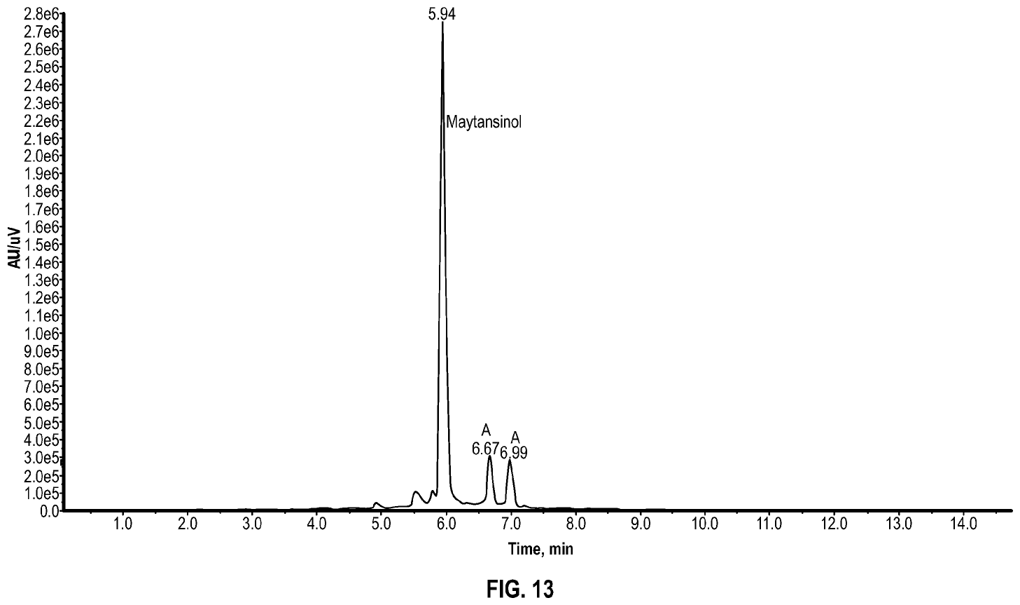

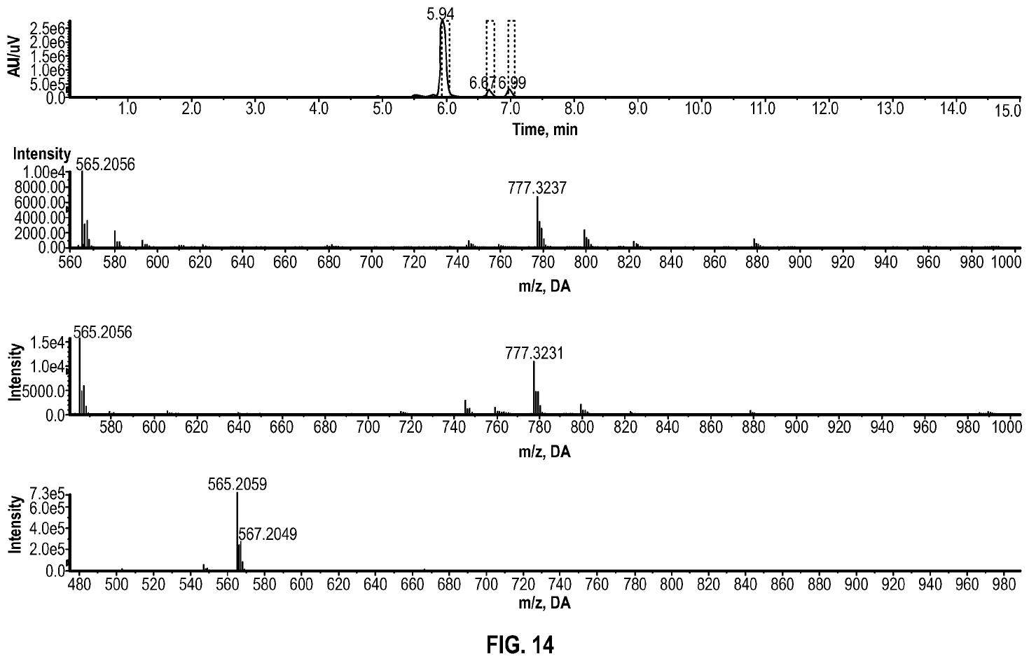

Maytansinol (4 mg, 0.0071 mM) was dissolved in acetonitrile 1 mL, and then mixed with bifunctional linker II (3.4 mg, 0.0142 mM). The mixture was irradiated by UV light (365 nm) for 3 hours at room temperature to generate maytansinol-linker II conjugates, which comprise at least two isomeric compounds having MS (ESI (m/z) [M+H]⁺) of 777.3 (FIG. 13 and FIG. 14).

### Example 14

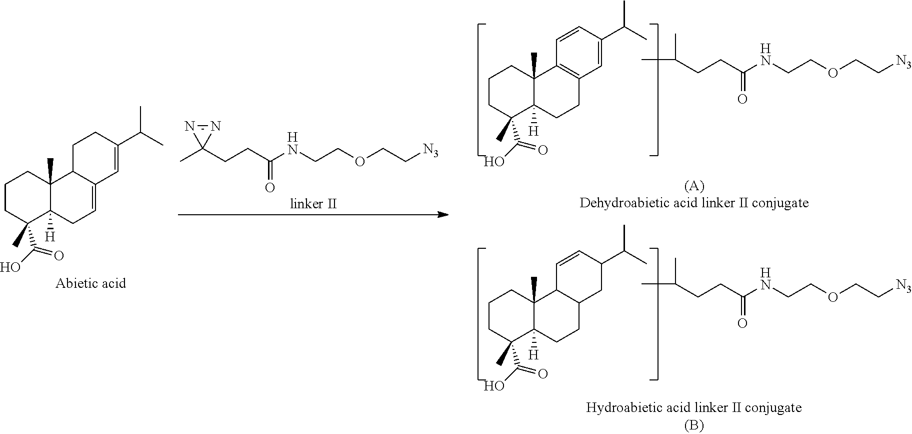

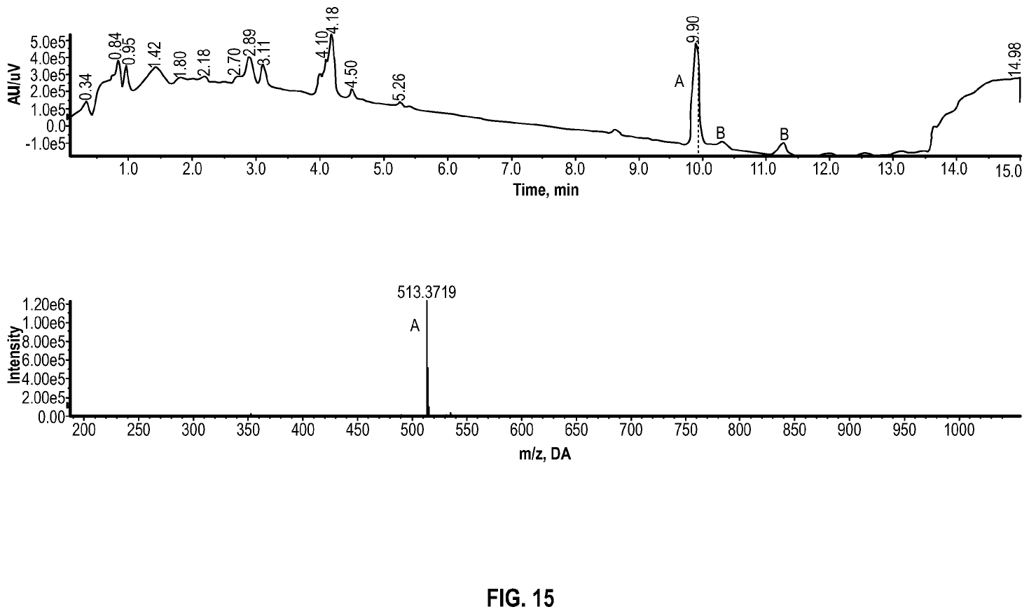

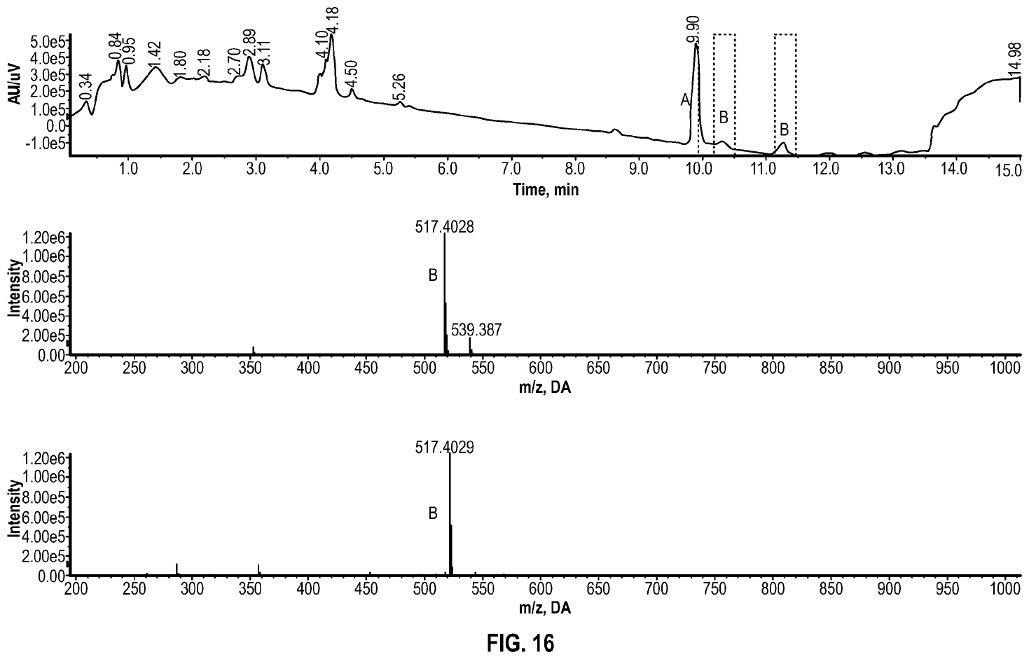

Abietic acid (2 mg, 0.0066 mM) was dissolved in 1 mL acetonitrile, and then mixed with bifunctional linker II (3.1 mg, 0.013 mM). The mixture was irradiated with UV light (365 nm) for 3 hours at room temperature. Two types of conjugates were generated (dehydroabietic acid linker II conjugate (A), having MS (ESI (m/z) [M+H]⁺) of 513.37, and hydroabietic acid linker II conjugate (B), having MS (ESI (m/z) [M+H]⁺) of 517.40). (FIG. 15 and FIG. 16).

### Example 15

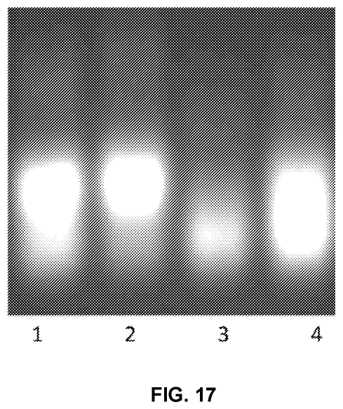

Reaction with oligonucleotides: compounds 6 and 7 were ligated with oligonucleotides using ligase. Headpiece-DNA conjugate 4 was mixed with photo sensitive linker 2. The mixture was irradiated by UV light (365 nm) for 2 hours. The product was ligated with oligonucleotide, the bands were smeared, indicating multiple ligated products were generated (FIG. 17).

### Example 16

Oligonucleotides that can be used as labels include single-stranded DNA (ssDNA), double-stranded DNA (dsDNA), single-stranded RNA (ssRNA), double-stranded RNA (dsRNA), chemical-modified oligonucleotides and some functional species such as antisense RNA (asRNA).

The sequences of several oligonucleotide examples for the labeling are listed as below:
ssDNA: 5′-AAATAAATT, 5′ amino modified
ssRNA: 5′-AUUUAUUUU, 5′ amino modified
ssDNA: 5′-AAATAAATT, 3′ amino modified
ssRNA: 5′-AUUUAUUUU, 3′ amino modified
ssDNA: 5′-AAATAAATT, 5′ amino modified with phosphorothioate linkages (which are resistant to degradation by nucleases).
ssDNA: 5′-GCGTTTGCTCTTCTTCTTGCG, 5′ amino modified with phosphorothioate linkages (SEQ ID NO:1) (which are resistant to degradation by nucleases).

### Example 17

DNA encoded chemical libraries (DELs) link the powers of genetics and chemical synthesis via combinatorial optimization. Through combinatorial chemistry, DELs can grow to the unprecedented size of billions to trillions, providing a rich chemical diversity for biological and pharmaceutical research. While in most cases at the molecular level, the diversity is confined to available building blocks of DNA compatible chemical reactions, modern chemical methods are now being used to increase the diversity. To take full advantage of the DEL approach, linking the power of genetics directly to chemical structures would offer even greater diversity in a finite chemical world. Natural products have evolved an incredible structural diversity along with their biological evolution.

The following provides an exemplary DNA encoded chemical library (DEL) using natural products, FDA approved drugs, compounds in clinical trials, and compounds from combinatorial synthesis, which was prepared according to the methods described herein. In the following example, the volatile bi-functional linker (linker IV) allowed “one-pot” reactions on an automated parallel synthesizer.

In some embodiments, methods described herein exhibit the following criteria: (1) site-nonselective, (2) chemo-nonselective, (3) biologically compatible (e.g., DNA-compatible), and (4) compatible with small reaction scales (e.g., microgram). The conventional chemo-selective reaction modifies natural products on one particular atom, as a result of spatial shielding, potential binding pockets that regulate functions of target proteins could be missed. A late stage modification method was designed targeting all accessible atoms using chemo- and site-nonselective reactions. Such late stage modifications yielded a cluster of isomers with a unique DNA tag, which provides multiple steric accessibilities to target proteins.

### General Methods

All commercially available organic compounds and DNA headpiece (HP-NH₂, 5′-/5phos/GAGTCA/iSp9/iUniAmM/iSp9/TGACTCCC-3′) were obtained from commercial sources unless otherwise noted. Unless otherwise noted, all commercial reagents and solvents were used without additional purification. NMR spectra were recorded on Bruker AM-500 NMR spectrometers. Chemical shifts were reported as δ (ppm) and coupling constants were reported as J (hertz). Tetramethylsilane (TMS) was used as an internal reference for ¹H NMR and CDCl₃ was used as an internal reference for ¹³C NMR (δ 77.0 ppm). Mass spectra were recorded on an AB SCIEX 4600 mass spectrometer or on a waters SQD 2 mass spectrometer. Linker IV was prepared according to Example 4.

### Linker Screening

Various bi-functional linkers as described herein were examined to develop a high-throughput DNA annotation strategy for natural products. The reactivity of functional groups on the linkers were orthogonal, where one terminal was designed for chemo- and site-nonselective reactions reactions and the other was employed to conjugate the DNA tag via copper (I)-catalyzed azide-alkyne cycloaddition (CuAAC) as described herein.

Exemplary bifunctional linkers were examined using oridonin as a model substrate (Table 1A), where oridonin (1 equivalent) and bifunctional linker (2 equivalent) were dissolved in acetonitrile and subjected to UV irradiation at room temperature.

As shown in Table 1A, the 3-(trifluoromethyl)-3H-diazirin-3-yl containing linker III yielded three isomers with 20% yield (based on oridonin concentration). The 3-methyl-3H-diazirin-3-yl containing linker II afforded six labeled oridonin isomers in a labeling efficiency of 69%, two of them having one oridonin labeled with two molecules of linker II. The 3-methyl-3H-diazirin-3-yl containing linker, 3-(2-azidoethyl)-3-methyl-3H-diazirine (linker IV) yielded one isomer. After the completion of reaction between linker IV and oridonin, the unreacted linker IV and/or by-products of linker IV were removed by vacuum as indicated by ¹H-NMR.

TABLE 1A
Labelling efficiency of different carbene or radical generation systems.

| | Structure of | Total | | |
|---|---|---|---|---|
| Linker | Bifunctional Linkers | conversion ^{a} | Isomers ^{b} | Isomers ^{c} |
| II | 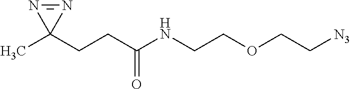 [SMILES: CC1(CCC(=O)NCCOCCN=[N+]=[N-])N=N1] | 69% | 4 | 2 |
| III | 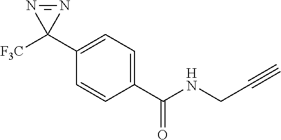 [SMILES: C#CCNC(=O)c1ccc(C2(C(F)(F)F)N=N2)cc1] | 20% | 3 | 0 |
| IV | 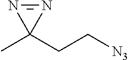 | 55% | 1 | trace |

^{a} To a solution of oridonin in dry acetonitrile 0.5 mL (0.1 mM) was added the relative bifunctional linker 1 mL (0.1 mM), and the resulting mixture was irradiated with a 365 nM lamp for 30 min, then the mixture was analyzed by LC-MS.
^{b} Oridonin labeled with one linker.
^{c} Oridonin labeled with two linkers.
^{d} No reaction.

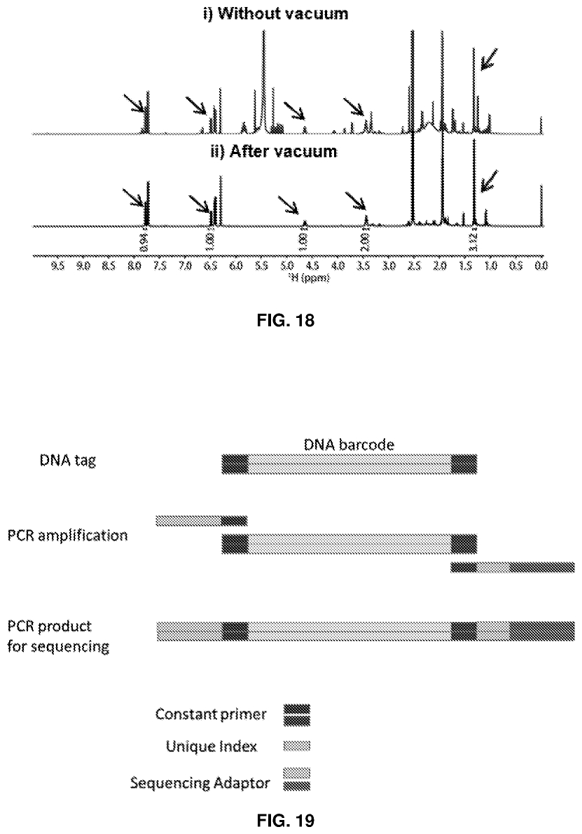

As shown in Table 1B by the reaction of linker IV with 2,4-dihydroxyacetophenone at a concentration as high as five equivalents, linker IV did not react with the azide functionality. As shown in FIG. 18, after the completion of reaction, unreacted linker IV and/or by-products of linker IV were readily removed by vacuum as indicated by ¹H-NMR (linker IV-2,4-dihydroxyacetophenone conjugates identified with an arrow). Trace amounts of remaining linker IV were shown having no interference with the subsequent DNA conjugation via CuAAC-based click chemistry and enzymatic DNA ligation.

TABLE 1B
Labelling Efficiency of Linker IV

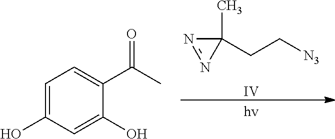

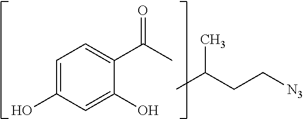

| | Ratio of | Total | | |
|---|---|---|---|---|
| Entry | linker IV | Conversion ^{a} | Isomers ^{b} | Isomers ^{c} |
| 1 | 0.5 | 15% | 1 | — |
| 2 | 1 | 26% | 1 | — |
| 3 | 2 | 55% | 1 | trace |
| 4 | 5 | 70% | 2 | 11% |

^{a} To a solution of 2,4-Dihydroxyacetophenone in dry acetonitrile 0.5 mL (0.1 mM) was added relative amounts of bifunctional linker IV, and the resulting mixture was irradiated with a 365 nM UV lamp for 30 min, then the mixture was analysis by LC-MS and ¹H NMR.
^{b} 2,4-Dihydroxyacetophenone labeled with one linker IV.
^{c} 2,4-Dihydroxyacetophenone labeled with two linker IV.

Linker conjugates shown in Table 2 were prepared using linker IV.

TABLE 2

| Organic | | | |
|---|---|---|---|
| compound | Structure | Yield^{a} | Isomers^{b} |
| 2,4- Dihydroxy- acetophenone | 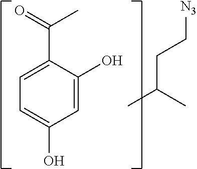 | 70% | 2 |
| Oridonin | 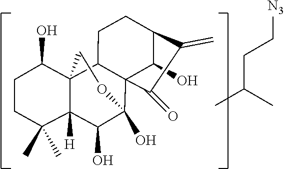 | 64% | 3 |
| Celastrol | 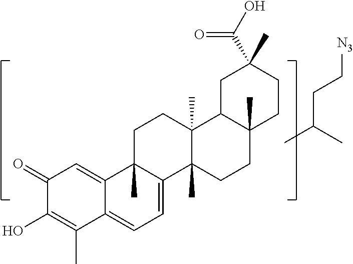 | 82% | 2 |
| Theophyline | 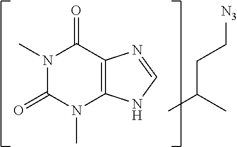 | 53% | 2 |
| Luteolin | 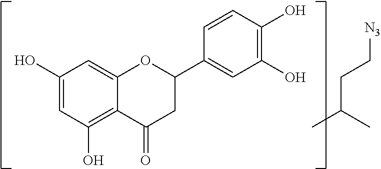 | 20% | 4 |
| Enoxolone | 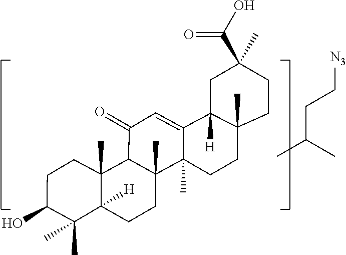 | 73% | 2 |
| Kinetin | 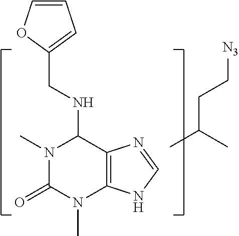 | 21% | 2 |
| Quercetin | 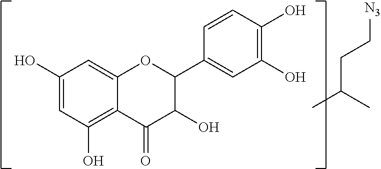 | 14% | 5 |
| Picrpside | 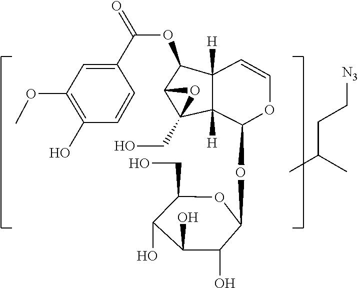 | 58% | 1 |

^{a}Yields were determined by LC.
^{b}Labeled isomers were determined by LC and MS-MS.

Scheme 2 shows the preparation of propyne-HP-DNA from propyne-(PEG)₅-CH₂CH₂COOH via amide coupling chemistry promoted by DMTMM for reacting with drugs and natural products.

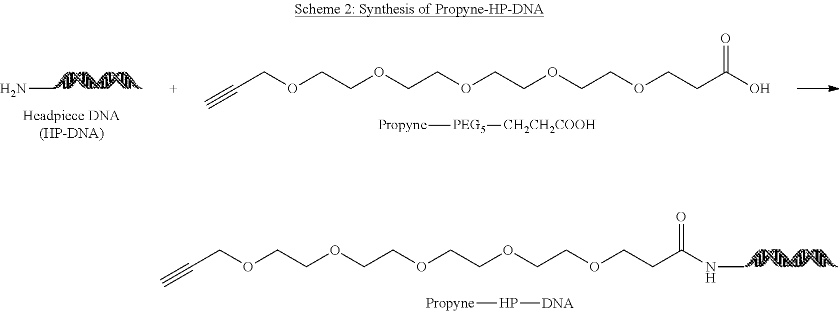

In Scheme 2, HP-DNA (1 mM) in pH 9.5 sodium borate buffer (250 mM), was added 40 equivalents of propyne-(PEG)₅-CH₂CH₂COOH (200 mM in DMF), followed by 50 equivalents of DMTMM (200 mM in water). After stirring at room temperature for 18 h, the reaction mixture was added 5 M NaCl solution (10% by volume) and cold ethanol (2.5 fold by volume, ethanol stored at −20° C.). The mixture was stored at −80° C. for more than 30 minutes. After that, the mixture was centrifuged for 15 minutes at 4° C. in a microcentrifuge at 12000 rpm. The supernatant was removed and the pellet was dissolved in water to the final concentration of 1 mM and used directly for the next step of click coupling reaction without further purification. Calculated Exact Mass: 5267.07. Found Mass: 5266.46.

A general procedure for the labelling of various drugs using bifunctional linker IV is as follows in Scheme 3. In Scheme 3, NP is a drug or natural product and propyne-HP-DNA is as described above. The compounds in Table 3 and Table 4 were prepared using these methods.

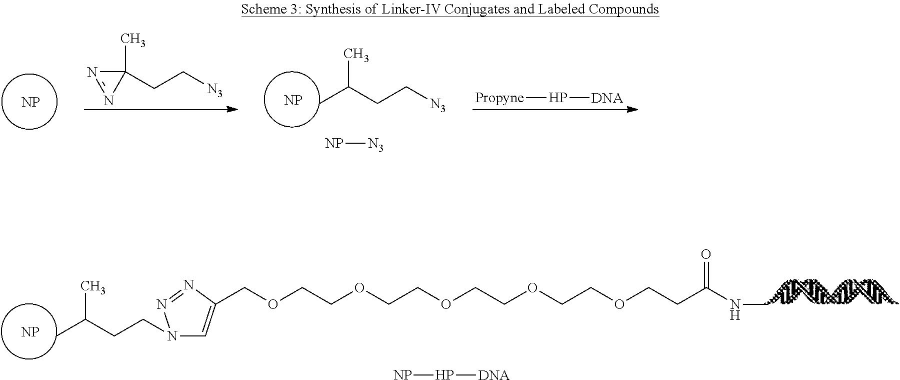

Briefly, CH₃CN (100 μL) were added to each well of a 96-well plate containing compounds (1 μmol) in each well and linker IV (5 μmol). The plate was irradiated under UV with a wavelength of 365 nm for 30 min at room temperature. The products and yields were evaluated by LC-MS. The CH₃CN was evaporated in vacuo overnight to generate the relative NP-N₃. Afterwards, the compounds (NP-N₃) were dissolved in DMSO (30 μL), and mixed with propyne-HP-DNA (10 μL, 1 mM in water), THPTA (10 μL, 80 mM in DMSO), CuSO₄.5H₂O (10 μL, 80 mM in water) and sodium ascorbate (20 μL, 80 mM in water). The resulting mixture was shaken at room temperature overnight, and the products and yields were evaluated by LC-MS upon the reaction finished. After that, the scavenger sodium diethyldithiocarbamic acid (12 μL, 160 mM in water) was added. Then all the HP-DNA conjugated compounds (NP-HP-DNA) were collected and added 5 M NaCl solution (10% by volume) and cold ethanol (2.5 times by volume, ethanol stored at −20° C.). The mixture was stored in a −80° C. freezer for more than 30 minutes. The mixture was centrifuged for 15 minutes at 4° C. in a microcentrifuge at 12000 rpm. The supernatant was removed and the pellet was dissolved in water.

DNA encoding was carried out in a 96-well plate using “one pot” stepwise synthesis as described herein. The drug-linker IV conjugates and labelled compounds shown in Table 3 and Table 4 were prepared according to the procedures described above. A total of 110 DNA encoded end-products were obtained (Table 4). For compounds with multiple functional groups, including one or more of hydroxyl, carboxyl, amine, etc., such as compound numbers 17, 25, 74, 82, 108, 91, 99, and 114, DNA conjugation occurred readily at multiple sites as indicated by HPLC fractions at different retention times showing the same molecular weight, whereas for compounds with single functional group, DNA conjugation at a single site was generally observed.

TABLE 3

| | | Structure of Drug-Linker IV | | MS |
|---|---|---|---|---|
| No. | Drug (NP) | Conjugate (NP-N₃) | HPLC-UV | [M + H]⁺ |
| 11 | Oridonin | 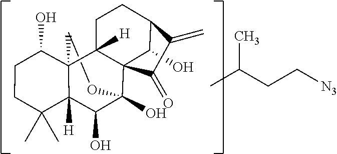 | retention time: t = 1.17, t = 1.46, t = 1.55, t = 1.59 | 462.5 |
| 12 | Myricetin | 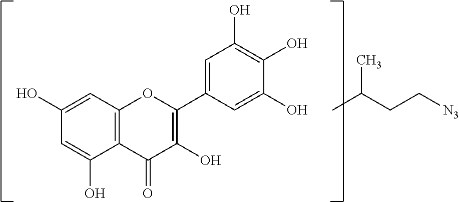 | retention time: t = 1.38, t = 1.42, t = 1.48, t = 1.51, t = 1.60 | 416.3 |
| 13 | Baicalein | 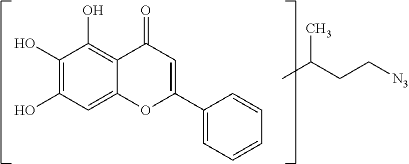 | retention time: t = 1.66, t = 1.88, t = 1.98, | 368.3 |
| 14 | Theophylline | 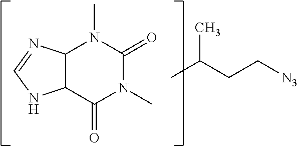 | retention time: t = 0.46, t = 1.01, | 278.3 |
| 15 | Kinetin | 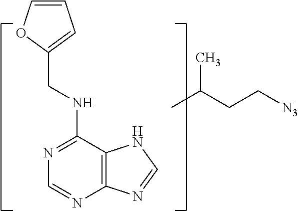 | retention time: t = 1.18, t = 1.27, t = 1.39 | 313.3 |
| 16 | Acetaminophen | 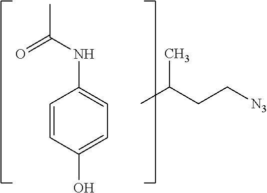 | retention time: t = 1.21, t = 1.43. | 249.3 |
| 17 | Protocatechuic acid | 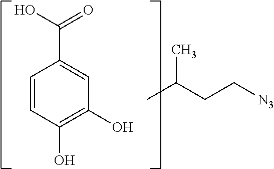 | retention time: t = 1.40, t = 1.79, | 250.3 |
| 18 | Bengenin | 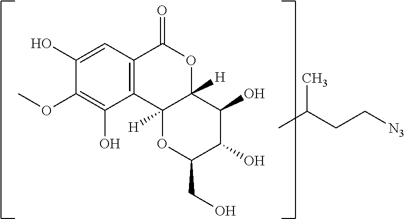 | retention time: t = 1.22 | 426.3 |
| 19 | Naringin | 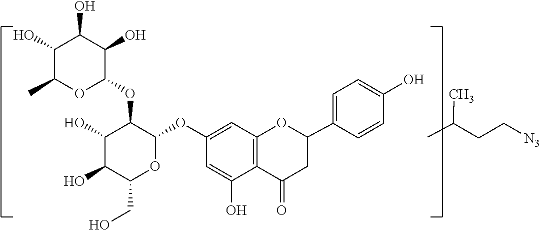 | retention time: t = 1.49 | 678.5 |
| 20 | Pyridoxine | 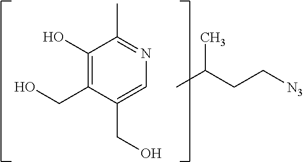 | retention time: t = 0.32, t = 0.54. | 267.3 |
| 21 | Methyl protocatechuate | 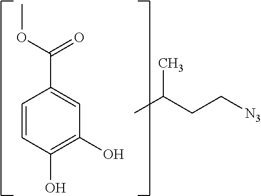 | retention time: t = 1.64, t = 1.68. | 264.3 |
| 22 | Theophylline-7- acetic acid | 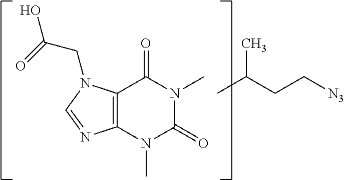 | retention time: t = 1.28. | 336.3 |
| 23 | Biochanin A | 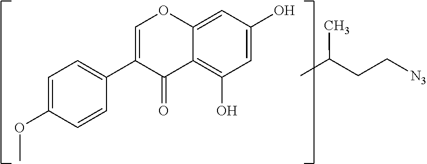 | retention time: t = 2.01, t = 2.05, t = 2.16. | 382.3 |
| 24 | Phloracetophenone | 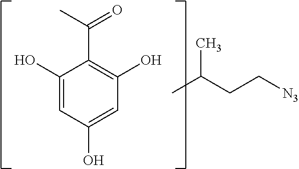 | retention time: t = 1.66, t = 1.70. | 266.3 |
| 25 | Gentisic acid | 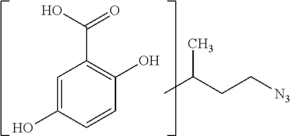 | retention time: t = 1.58, t = 1.62, t = 1.65. | 250.3 |
| 26 | Theobromine | 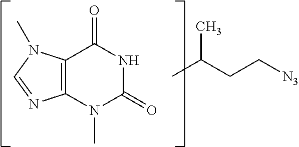 | retention time: t = 0.77, t = 0.88, t = 1.15. | 278.3 |
| 27 | Luteolin | 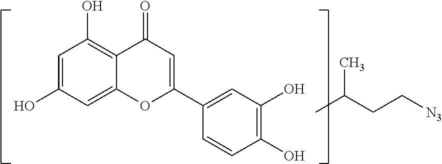 | retention time: t = 1.64, t = 1.67, t = 1.74, t = 1.76. | 384.3 |
| 28 | Nicotinic acid | 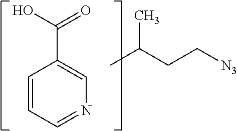 | retention time: t = 1.38. | 221.3 |
| 29 | Esculetin | 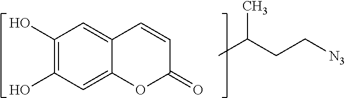 | retention time: t = 1.39, t = 1.44. | 276.3 |
| 30 | Phlorizin | 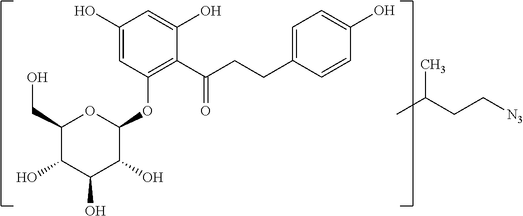 | retention time: t = 1.26, t = 1.45. | 534.5 |
| 31 | Picroside II | 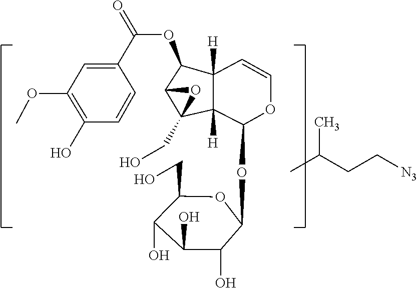 | retention time: t = 1.41. | 600.5 |
| 32 | 7-Hydroxycoumarin | 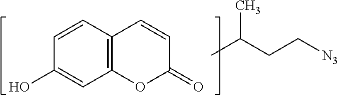 | retention time: t = 1.73. | 260.3 |
| 33 | Nocodazole | 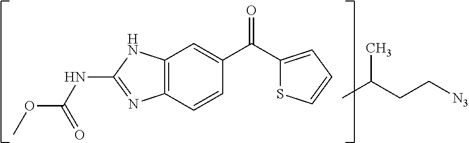 | retention time: t = 1.73, t = 1.79. | 399.3 |
| 34 | Scopoletin | 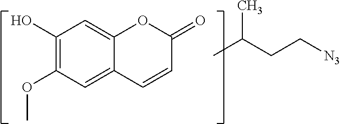 | retention time: t = 1.61. | 290.3 |
| 35 | Jatrorrhizine | 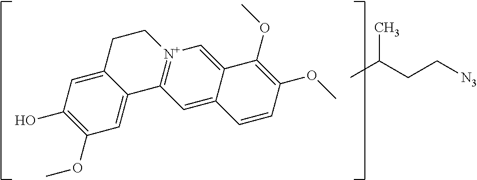 | retention time: t = 1.79. | 435.4 |
| 36 | Fraxetin | 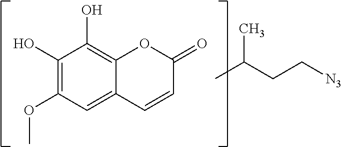 | retention time: t = 1.45, t = 1.55. | 306.3 |
| 37 | Daphnetin | 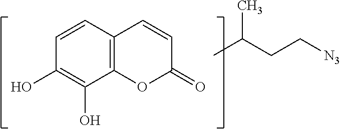 | retention time: t = 1.46, t = 1.50. | 274.3 |
| 38 | Quercetin | 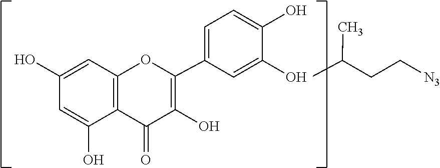 | retention time: t = 1.38, t = 1.44, t = 1.52, t = 1.57, t = 1.60. | 400.2 |
| 39 | Hyperoside | 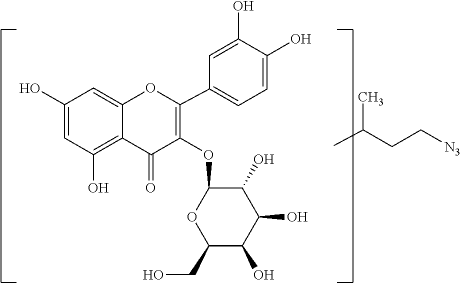 | retention time: t = 1.30, t = 1.34, t = 1.38, t = 1.48. | 562.4 |
| 40 | (−)- Epicatechingallate | 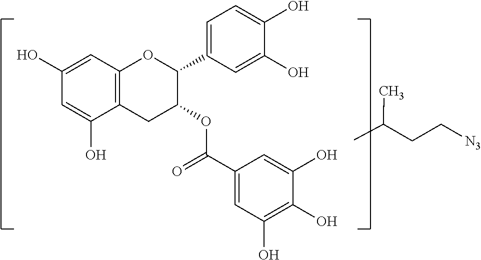 | retention time: t = 1.29, t = 1.35, t = 1.39, t = 1.41, t = 1.44. | 540.4 |
| 41 | Plumbagin | 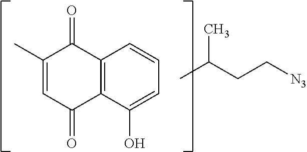 | retention time: t = 1.62. | 284.3 |
| 42 | Liquiritin | 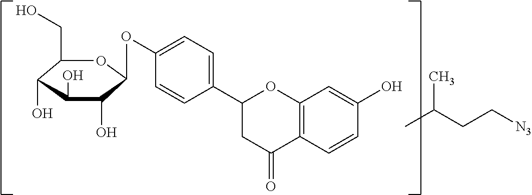 | retention time: t = 1.51. | 516.4 |
| 43 | Liquiritigenin | 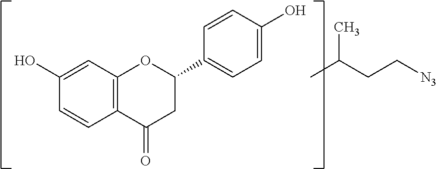 | retention time: t = 1.79. | 354.3 |
| 44 | Benzyladenine | 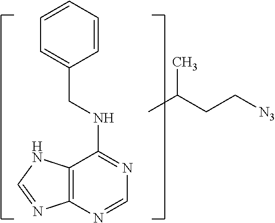 | retention time: t = 1.40, t = 1.54, t = 1.64. | 323.3 |
| 45 | Dihydromyricetin | 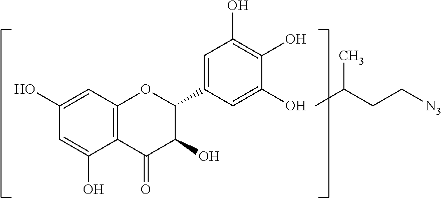 | retention time: t = 1.25, t = 1.42. | 418.1 |
| 46 | Cianidano | 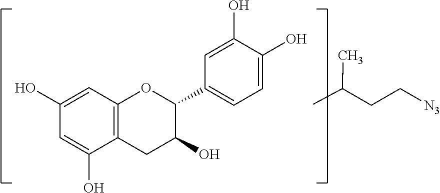 | retention time: t = 1.07, t = 1.22. | 388.2 |
| 47 | Silibinin | 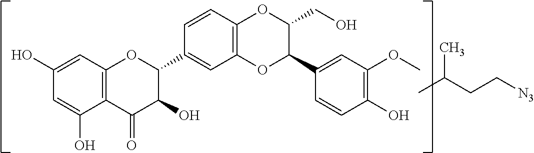 | retention time: t = 1.69, t = 1.71, t = 1.73. | 580.2 |
| 48 | (−)-Gallocatechin gallate | 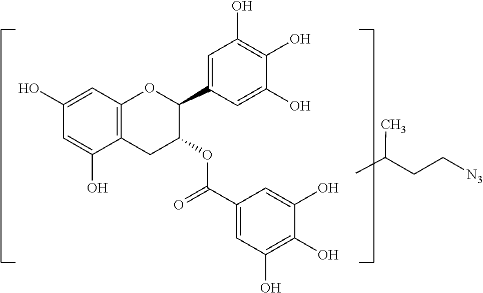 | retention time: t = 1.11, t = 1.21. | 554.2 |
| 49 | Tolcapone | 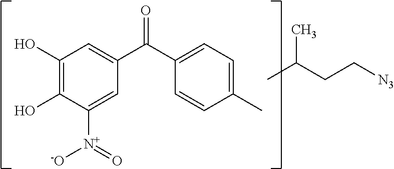 | retention time: t = 1.95, t = 1.98. | 369.3 |
| 50 | Cinchophen | 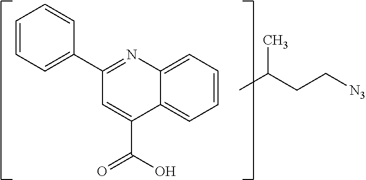 | retention time: t = 2.25. | 347.3 |
| 51 | Probenecid | 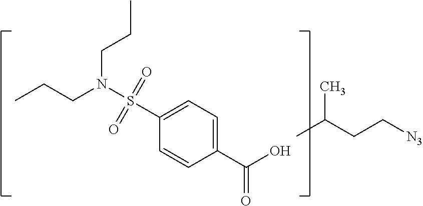 | retention time: t = 2.10. | 383.3 |
| 52 | Dexibuprofen | 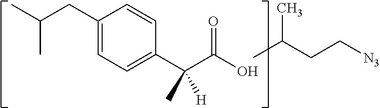 | retention time: t = 2.33. | 304.3 |
| 53 | Phthalylsulfacetamide | 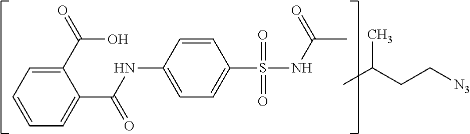 | retention time: t = 1.49, t = 1.58. | 459.2 |
| 54 | Indometacin | 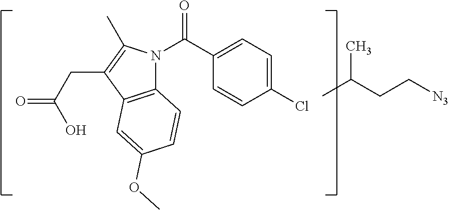 | retention time: t = 2.19. | 455.3 |
| 55 | Carzenide | 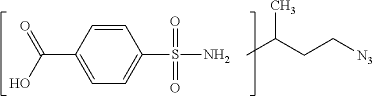 | retention time: t = 1.42, t = 1.46. | 297.3 |
| 56 | Sulindac | 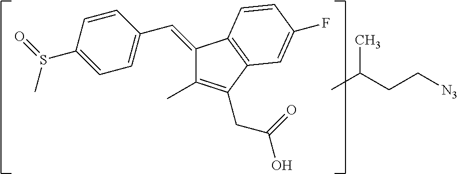 | retention time: t = 1.90. | 454.3 |
| 57 | ABT-492 | 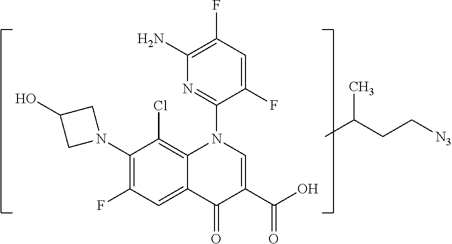 | retention time: t = 1.54. | 538.3 |
| 58 | Diclofenac | 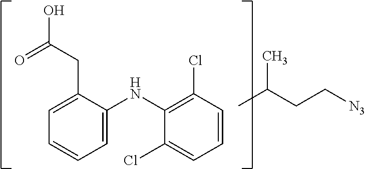 | retention time: t = 2.25. | 393.2 |
| 59 | Loxoprofen | 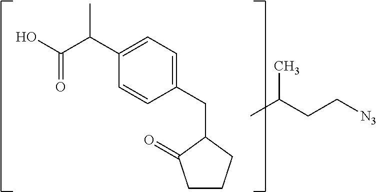 | retention time: t = 1.99. | 343.4 |
| 60 | Pefloxacin | 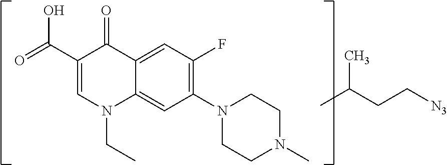 | retention time: t = 1.09, t = 1.23. | 431.3 |
| 61 | Cetirizine | 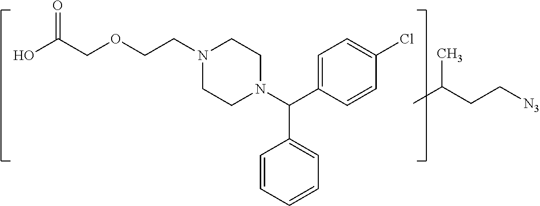 | retention time: t = 1.56. | 389.3 |
| 62 | Pranoprofen | 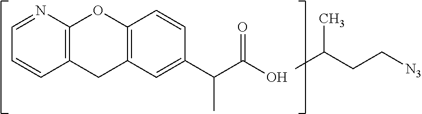 | retention time: t = 1.90. | 353.3 |
| 63 | Argatroban | 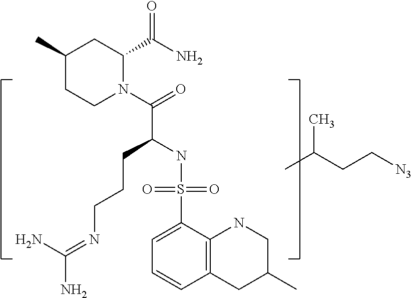 | retention time: t = 1.75. | 606.3 |
| 64 | Benazepril | 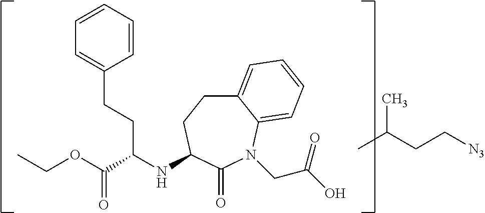 | retention time: t = 2.24. | 522.5 |
| 65 | Fenoprofen | 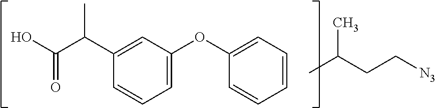 | retention time: t = 2.20. | 339.6 |
| 66 | iprofloxacin | 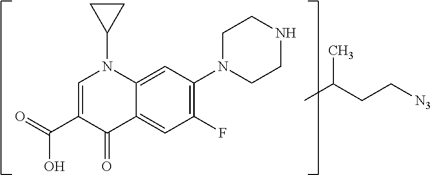 | retention time: t = 1.29. | 429.3 |
| 67 | Cinoxacin | 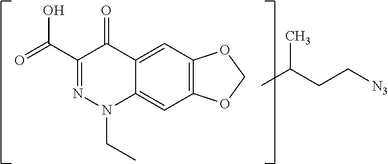 | retention time: t = 1.51. | 360.3 |
| 68 | Orbifloxacin | 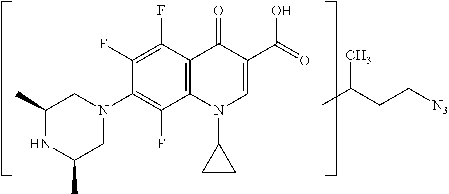 | retention time: t = 1.49. | 493.4 |
| 69 | Indoprofen | 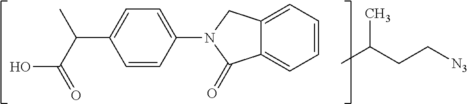 | retention time: t = 1.93. | 379.3 |
| 70 | Moxifloxacin | 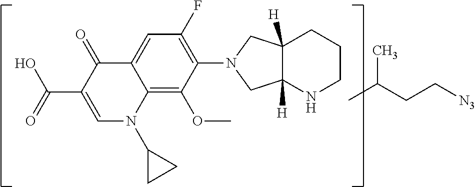 | retention time: t = 1.74. | 499.4 |
| 71 | Phthalylsulfathiazole | 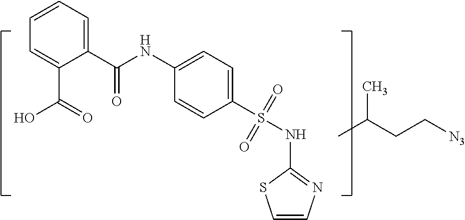 | retention time: t = 1.37, t = 1.63, t = 1.66. | 501.3 |
| 72 | Rabeprazole related compound E | 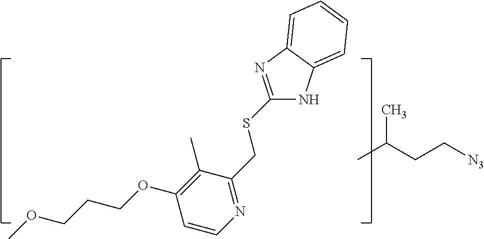 | rcrcntion time: t = 2.70 | 441.4 |
| 73 | Actarit | 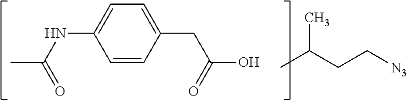 | retention time: t = 1.46, t = 1.50. | 291.3 |
| 74 | Azilsartan | 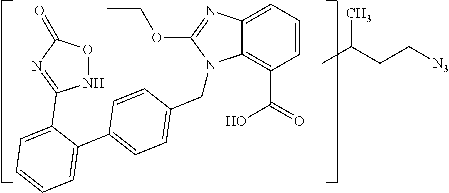 | retention time: t = 1.82, t = 1.95. | 554.5 |
| 75 | Flumequine | 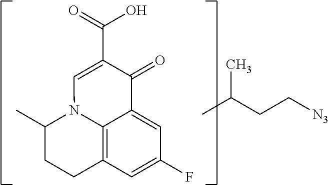 | retention time: t = 1.58. | 359.3 |
| 76 | Proglumide | 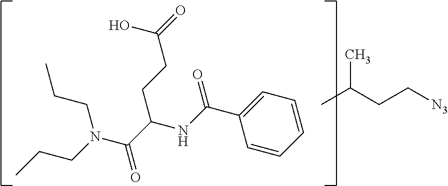 | retention time: t = 1.90, t = 1.92. | 432.4 |
| 77 | Sparfloxacin | 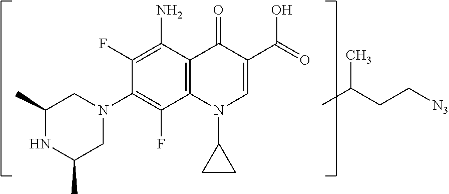 | retention time: t = 1.74. | 490.4 |
| 78 | Sarafloxacin | 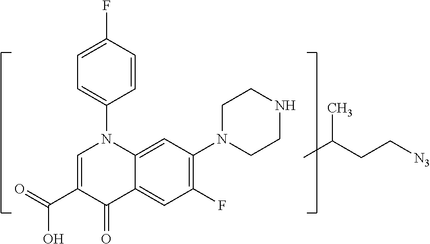 | retention time: t = 1.74. | 483.4 |
| 79 | Oxolinic acid | 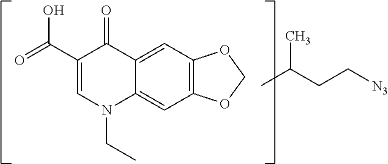 | retention time: t = 1.42. | 359.3 |
| 80 | Tazobactam acid | 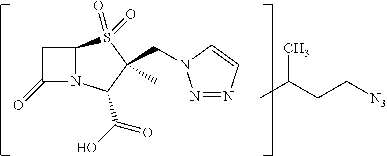 | retention time: t = 0.49. | 398.3 |
| 81 | Diacerein | 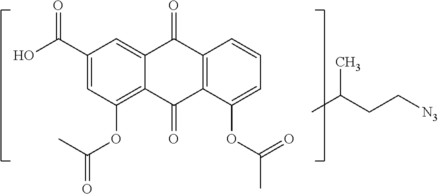 | retention time: t = 1.54. | 453.4 |
| 82 | Succinylsulfathiazole | 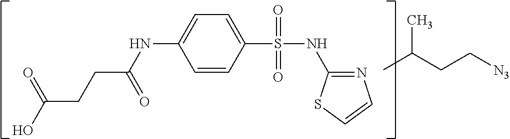 | retention time: t = 1.17, t = 1.36, t = 1.44, t = 1.51. | 453.3 |
| 83 | Ofloxacin | 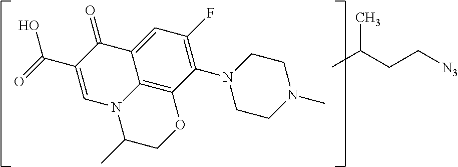 | retention time: t = 1.17. | 459.4 |
| 84 | Nalidixic acid | 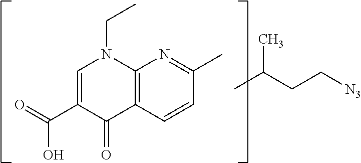 | retention time: t = 1.62. | 330.3 |
| 85 | Tetryzoline | 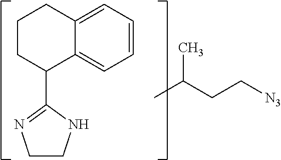 | retention time: t = 1.70. | 298.3 |
| 86 | Flubendazole | 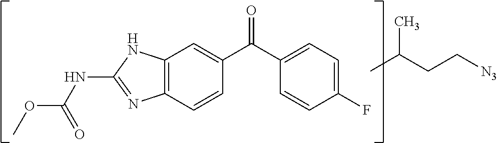 | retention time: t = 1.78, t = 1.85. | 411.3 |
| 87 | Phenytoin | 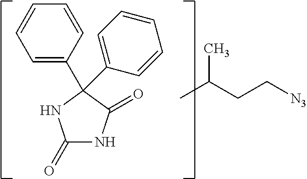 | retention time: t = 1.70, t = 1.74, t = 1.77. | 350.3 |
| 88 | Ziprasidone | 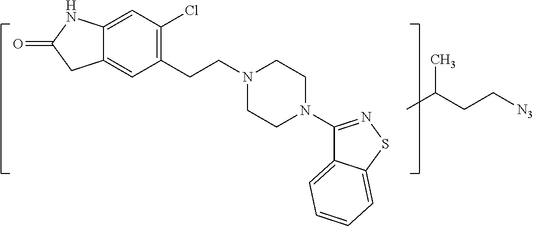 | retention time: t = 2.13. | 510.4 |
| 89 | Sulfalen | 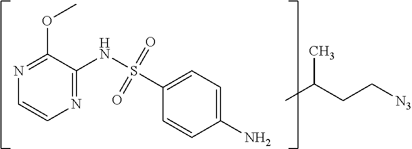 | retention time: t = 1.50, t = 1.53, t = 1.57. | 378.3 |
| 90 | Thalidomide | 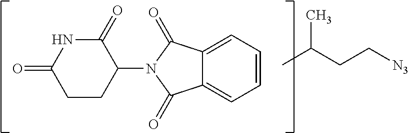 | retention time: t = 1.55. | 356.3 |
| 91 | Gemcitabine | 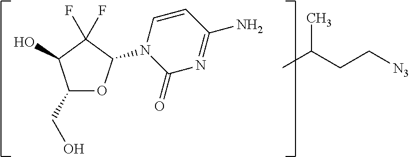 | retention time: t = 0.77, t = 1.11 t = 1.25. | 361.3 |
| 92 | Tizanidine | 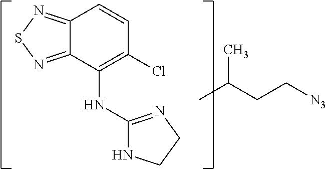 | retention time: t = 1.35. | 351.2 |
| 93 | Methylthiouracil | 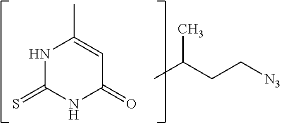 | retention time: t = 1.24, t = 1.35. | 240.2 |
| 94 | Rosiglitazone | 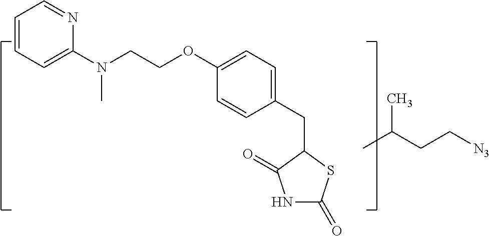 | retention time: t = 1.78. | 455.3 |
| 95 | Zileuton | 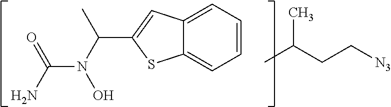 | retention time: t = 1.81. | 334.3 |
| 96 | Sunitinib | 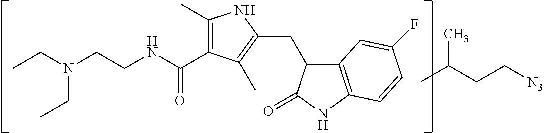 | retention time: t = 1.33, t = 1.61. | 496.5 |
| 97 | Losartan | 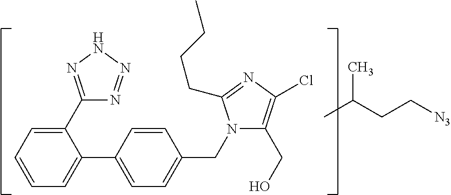 | retention time: t = 1.82, t = 2.00, t = 2.04. | 520.4 |
| 98 | Pioglitazone | 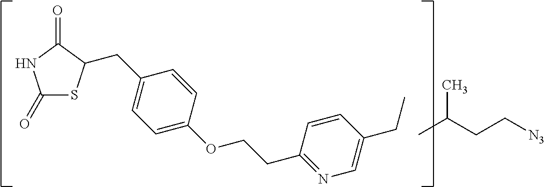 | retention time: t = 2.13. | 454.3 |
| 99 | Fluorocytosine | 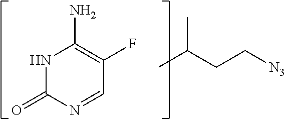 | retention time: t = 0.41, t = 0.82. | 227.3 |
| 100 | R-(+)-Lansoprazole | 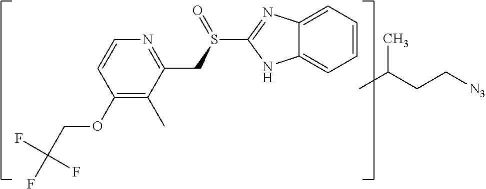 | retention time: t = 1.76, t = 1.79. | 467.3 |
| 101 | Zolmitriptan | 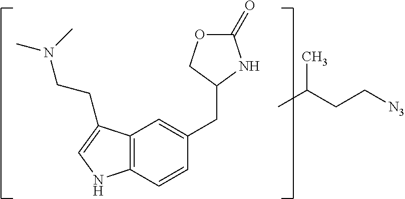 | retention time: t = 1.00. | 385.4 |
| 102 | Fenbendazole | 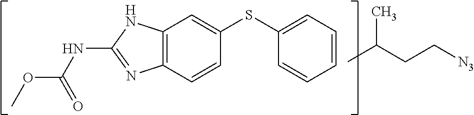 | retention time: t = 1.55, t = 2.05. | 397.3 |
| 103 | Albendazole | 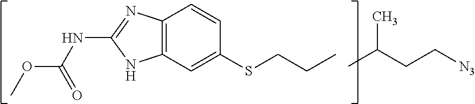 | retention time: t = 1.97. | 363.3 |
| 104 | Mebendazole | 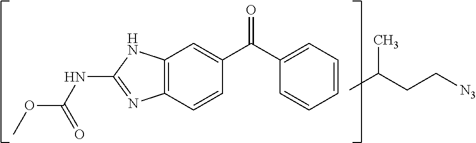 | retention time: t = 1.81, t = 1.85. | 393.3 |
| 105 | Tolazamide | 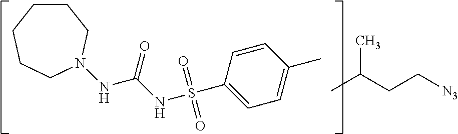 | retention time: t = 1.97, t = 2.02. | 409.3 |
| 106 | Azathioprine | 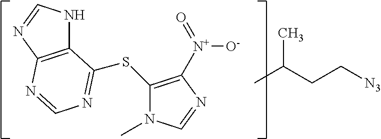 | retention time: t = 0.96, t = 1.16, t = 1.31. | 375.2 |
| 107 | Oxfendazole | 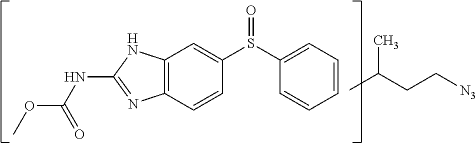 | retention time: t = 1.51, t = 1.54, t = 1.61. | 413.3 |
| 108 | Ganciclovir | 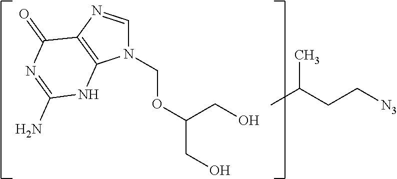 | retention time: t = 0.31, t = 0.68. | 353.3 |
| 109 | Allopurinol | 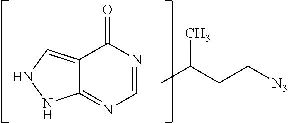 | retention time: t = 0.41, t = 0.63, t = 0.85, t = 1.33. | 234.3 |
| 110 | Lansoprazole | 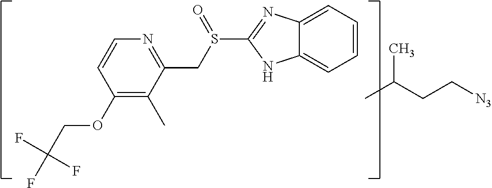 | retention time: t = 1.75, t = 1.80. | 467.3 |
| 111 | Omeprazole | 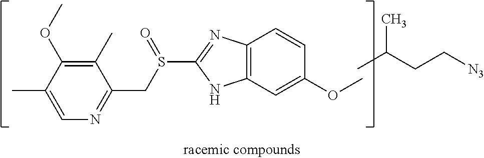 | retention time: t = 1.68. | 443.3 |
| 112 | Thiabendazole | 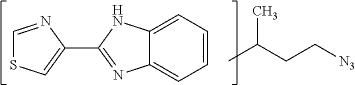 | retention time: t = 1.49. | 299.3 |
| 113 | Axitinib | 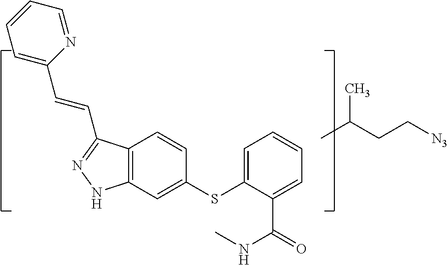 | retention time: t = 1.75. | 484.4 |
| 114 | Benicar | 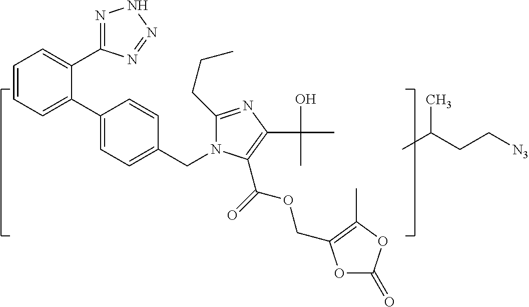 | retention time: t = 2.02, t = 2.45. | 656.6 |
| 115 | Omeprazole sulfide | 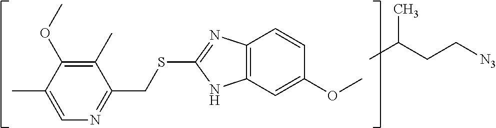 | retention time: t = 2.11. | 427.3 |
| 116 | Esomeprazole | 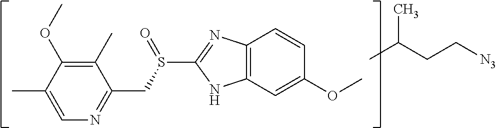 | retention time: t = 1.68. | 443.3 |
| 117 | Irbesartan | 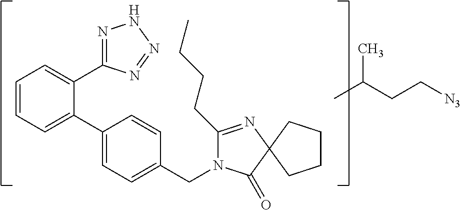 | retention time: t = 2.25. | 526.5 |
| 118 | Chlorzoxazone | 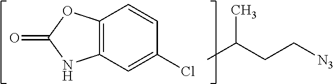 | retention time: t = 2.02. | 267.3 |
| 119 | Alizapride | 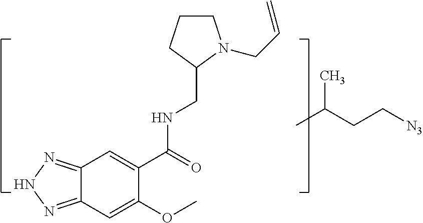 | retention time: t = 1.75. | 413.4 |
| 120 | Carbendazim | 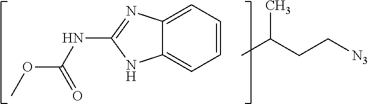 | retention time: t = 1.50, t = 1.53. | 289.3 |
| 121 | Topiroxostat | 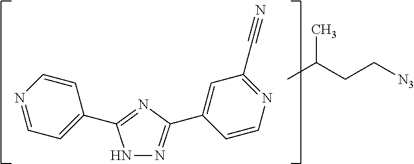 | retention time: t = 1.43, t = 1.58. | 346.3 |
| 122 | CAL-101 | 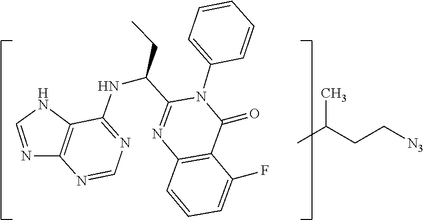 | retention time: t = 1.74, t = 1.80. | 513.4 |

TABLE 4

| Drug conjugate | | Calculated | MS |
|---|---|---|---|
| (NP-HP-DNA) | Structure of (NP-HP-DNA) | Mass | Found |
| Oridonin-HP-DNA | 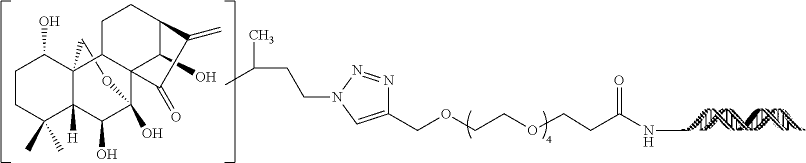 | 5728.32 | 5728.48 |
| Myricetin-HP-DNA | 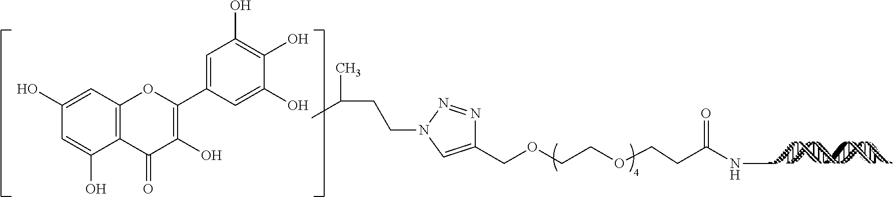 | 5682.17 | 5682.74 |
| Baicalein-HP-DNA | 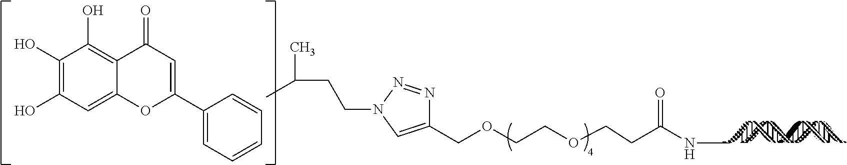 | 5634.18 | 5634.95 |
| Theophylline-HP- DNA | 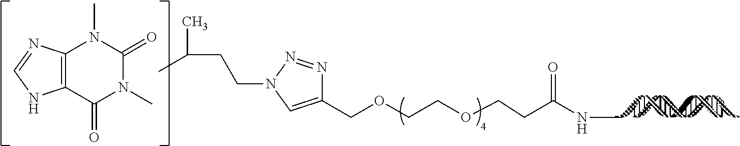 | 5544.19 | 5544.20 |
| Kinetin-HP-DNA | 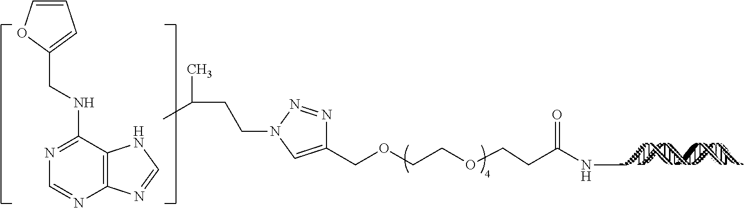 | 5579.21 | 5579.97 |
| Acetaminophen- HP-DNA | 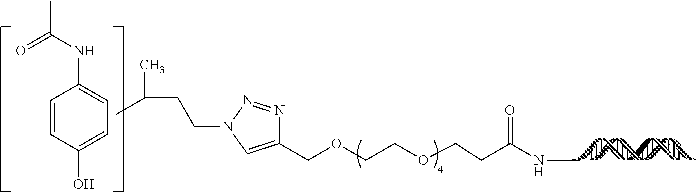 | 5515.19 | 5515.92 |
| Protocatechuic acid-HP-DNA | 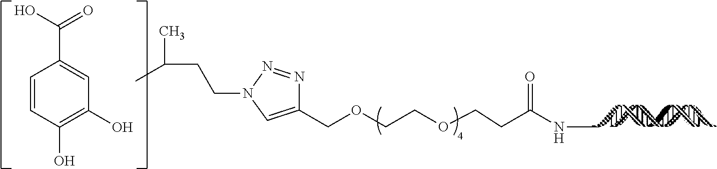 | 5518.16 | 5518.85 |
| Bengenin-HP-DNA | 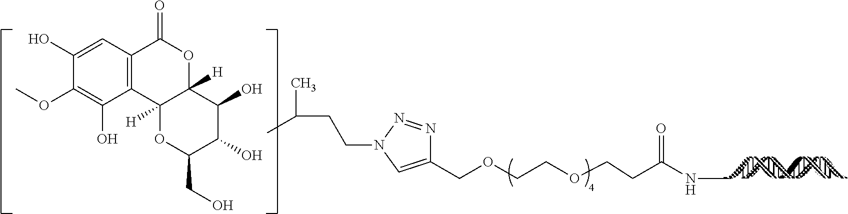 | 5692.21 | 5693.07 |
| Naringin-HP-DNA | 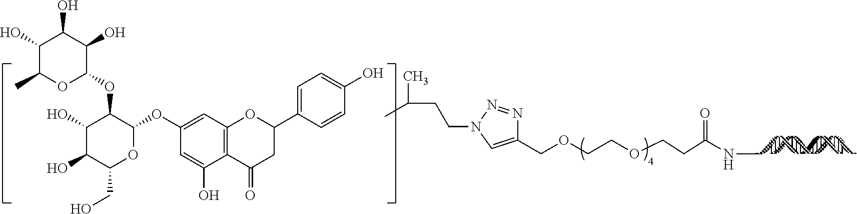 | 5944.31 | 5945.31 |
| Pyridoxine-HP- DNA | 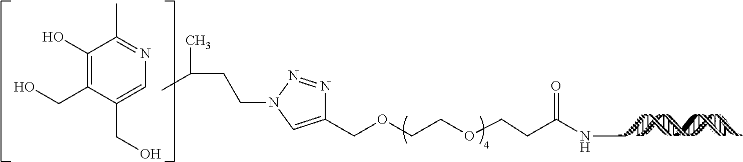 | 5533.20 | 5533.88 |
| Methyl protocatechuate- HP-DNA | 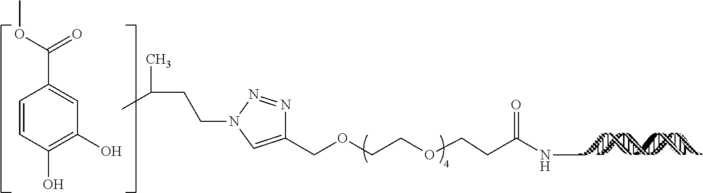 | 5532.17 | 5532.84 |
| Theophylline-7- acetic acid- HP-DNA | 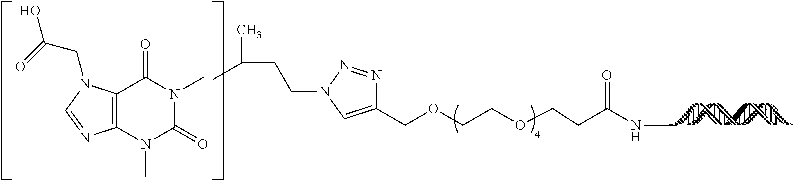 | 5602.20 | 5602.87 |
| Biochanin A-HP- DNA | 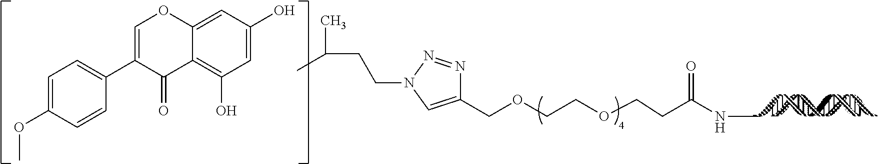 | 5648.20 | 5649.07 |
| Phloracetophenone- HP-DNA | 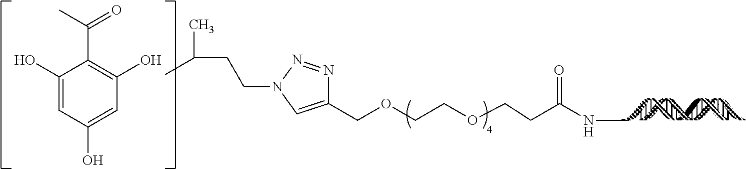 | 5532.17 | 5532.89 |
| Gentisic acid-HP- DNA | 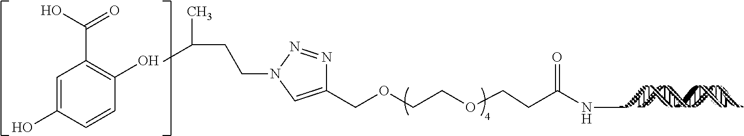 | 5518.16 | 5518.85 |
| Theobromine-HP- DNA | 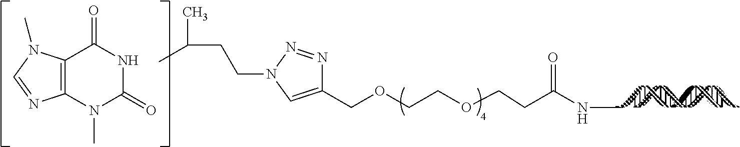 | 5544.19 | 5545.08 |
| Luteolin-HP-DNA | 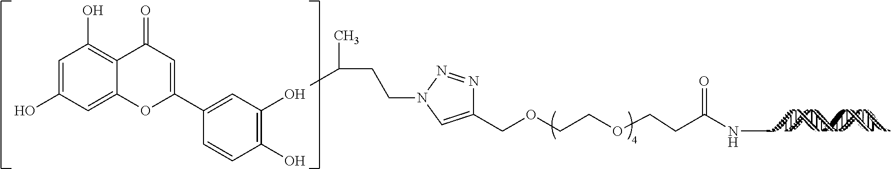 | 5650.18 | 5650.96 |
| Nicotinic acid-HP- DNA | 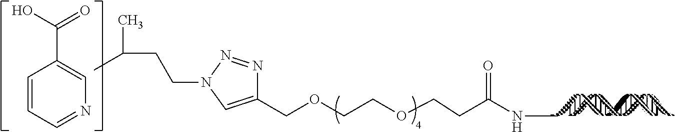 | 5487.16 | 5488.00 |
| Esculetin-HP-DNA | 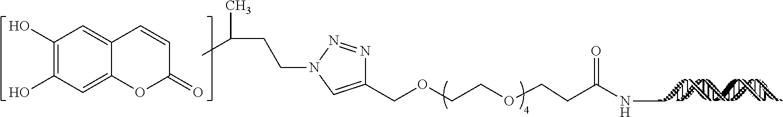 | 5542.16 | 5543.00 |
| Phlorizin-HP-DNA | 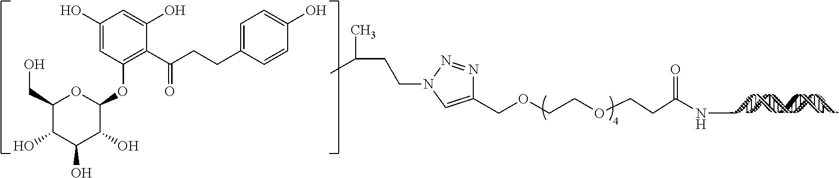 | 5800.27 | 5801.18 |
| Picroside II-HP- DNA | 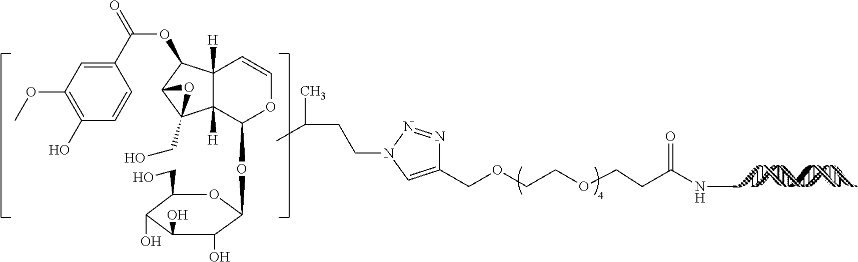 | 5876.28 | 5877.27 |
| 7- Hydroxycoumarin- HP-DNA | 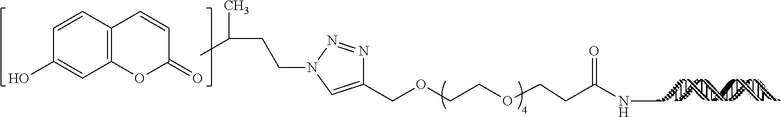 | 5526.16 | 5526.88 |
| Nocodazole-HP- DNA | 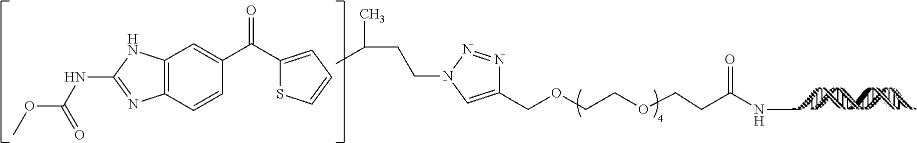 | 5665.18 | 5666.00 |
| Scopoletin-HP- DNA | 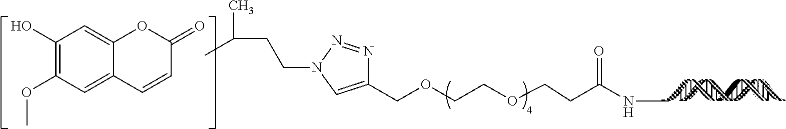 | 5556.17 | 5556.89 |
| Jatrorrhizine-HP- DNA | 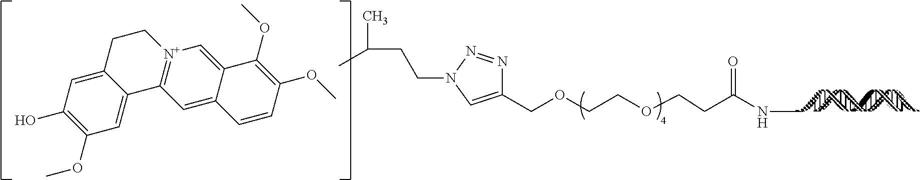 | 5702.27 | 5702.12 |
| Fraxetin-HP-DNA | 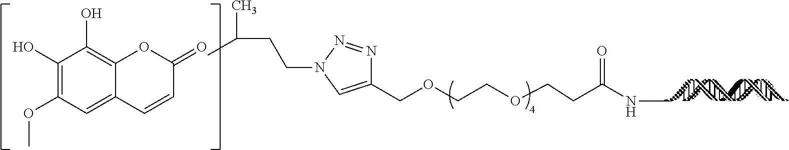 | 5572.17 | 5572.31 |
| Daphnetin-HP- DNA | 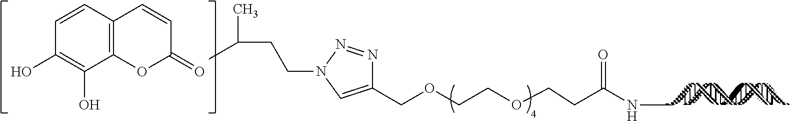 | 5542.16 | 5542.74 |
| Quercetin-HP-DNA | 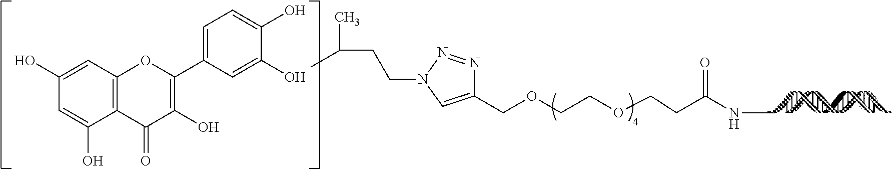 | 5666.17 | 5666.81 |
| Hyperoside-HP-DNA | 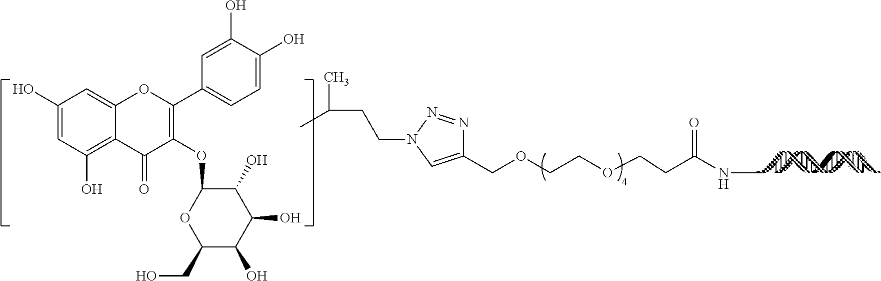 | 5828.23 | 5829.08 |
| (−)-Epicatechin gallate-HP-DNA | 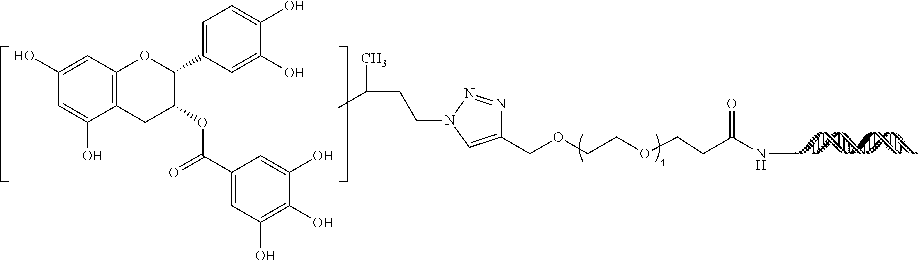 | 5806.22 | 5806.99 |
| Plumbagin-HP- DNA | 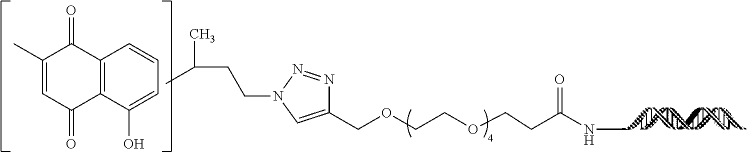 | 5522.18 | 5522.74 |
| Liquiritin-HP-DNA | 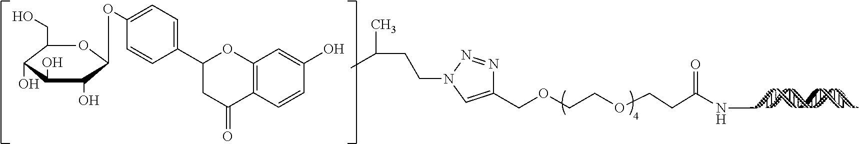 | 5782.26 | 5783.16 |
| Liquiritigenin-HP- DNA | 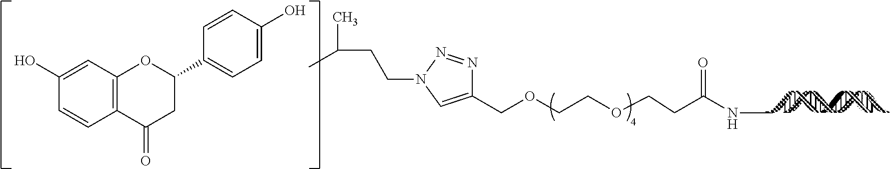 | 5620.20 | 5620.93 |
| Benzyladenine-HP- DNA | 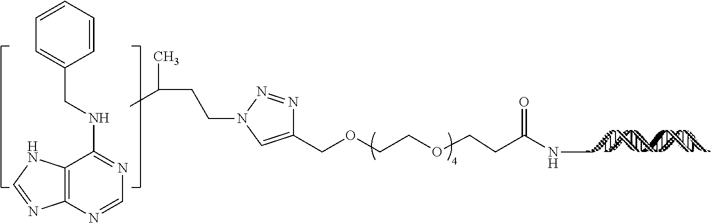 | 5589.23 | 5590.03 |
| Dihydromyricetin- HP-DNA | 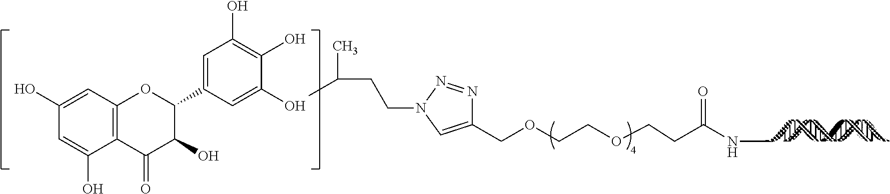 | 5684.18 | 5684.21 |
| Cianidanol-HP- DNA | 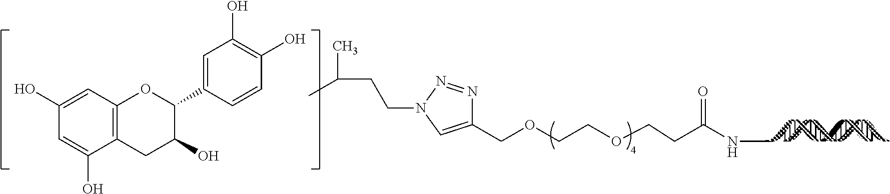 | 5654.21 | 5654.37 |
| Silibinin-HP-DNA | 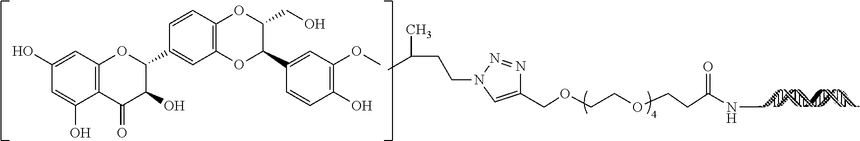 | 5846.25 | 5847.20 |
| (−)-Gallocatechin gallate-HP-DNA | 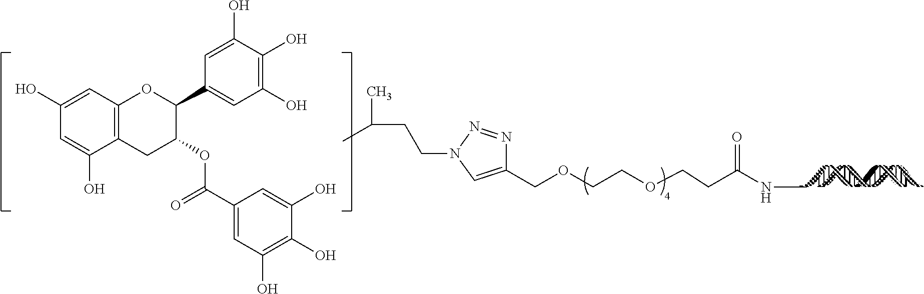 | 5822.21 | 5821.66 |
| Tolcapone-HP- DNA | 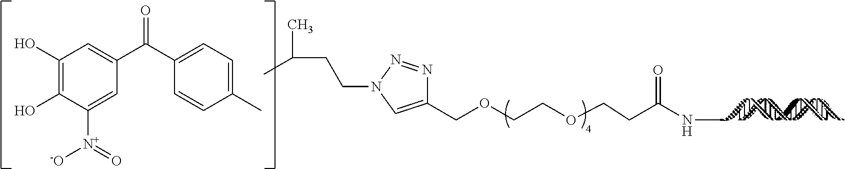 | 5637.19 | 5637.81 |
| Cinchophen-HP- DNA | 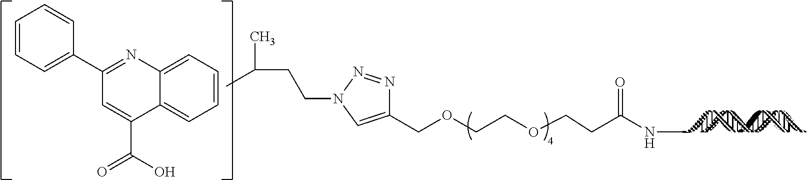 | 5613.21 | 5613.37 |
| Probenecid-HP- DNA | 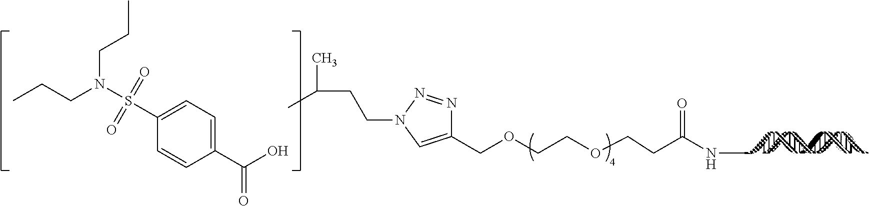 | 5649.23 | 5649.47 |
| Dexibuprofen-HP- DNA | 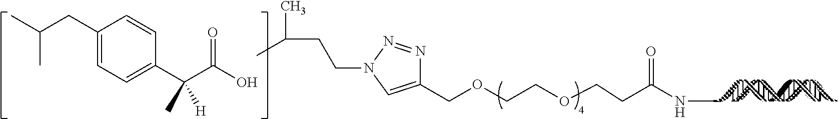 | 5570.26 | 5570.66 |
| Phthalylsulfacetamide- HP-DNA | 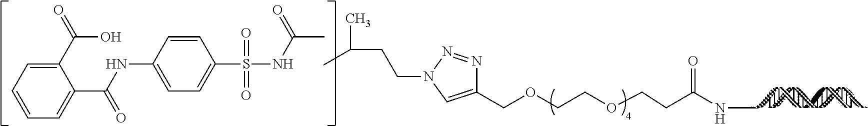 | 5726.19 | 5726.78 |
| Indometacin-HP-DNA | 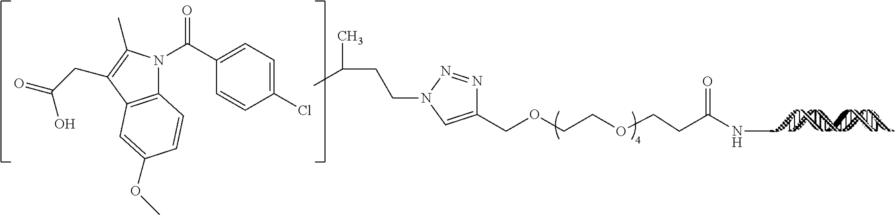 | 5721.21 | 5722.17 |
| Carzenide-HP- DNA | 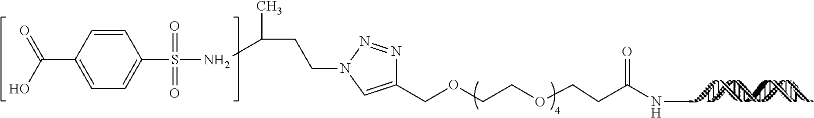 | 5565.14 | 5565.54 |
| Sulindac-HP-DNA | 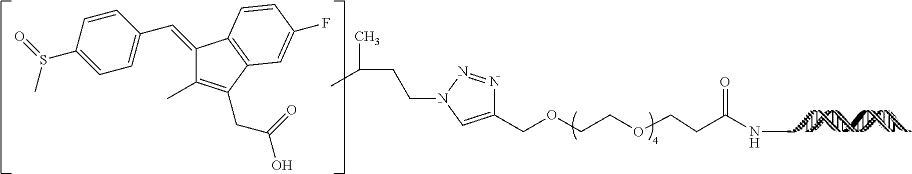 | 5720.22 | 5720.78 |
| ABT-492-HP-DNA | 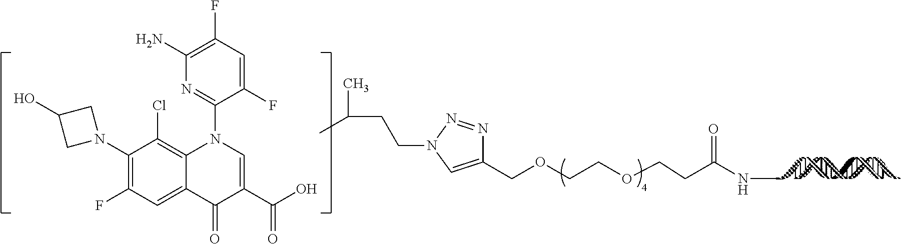 | 5804.18 | 5805.13 |
| Diclofenac-HP- DNA | 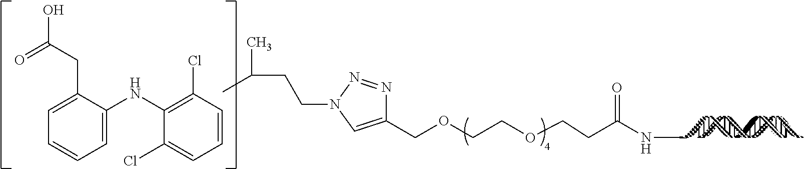 | 5659.15 | 5660.00 |
| Loxoprofen-HP- DNA | 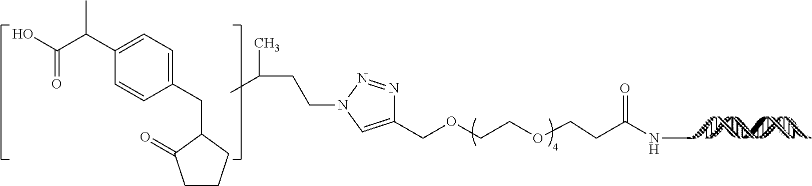 | 5610.26 | 5610.46 |
| Pefloxacin-HP-DNA | 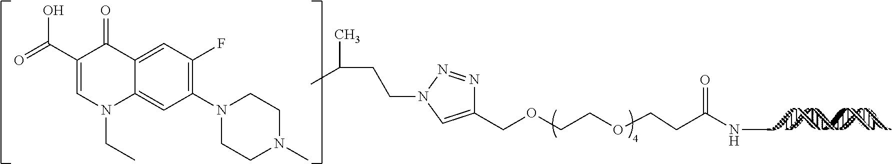 | 5697.28 | 5697.44 |
| Cetirizine-HP-DNA | 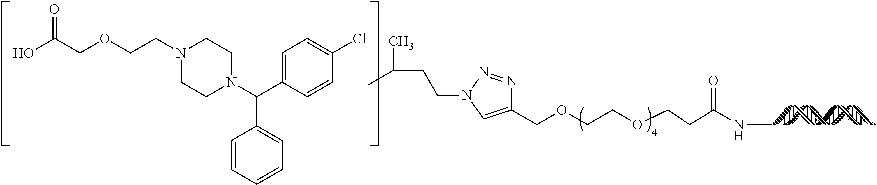 | 5752.29 | 5752.92 |
| Pranoprofen-HP- DNA | 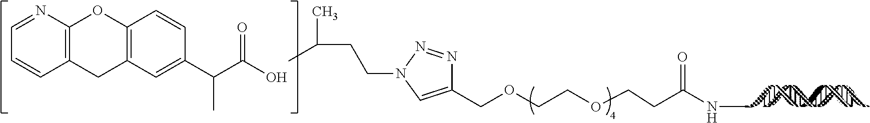 | 5619.22 | 5619.34 |
| Argatroban-HP- DNA | 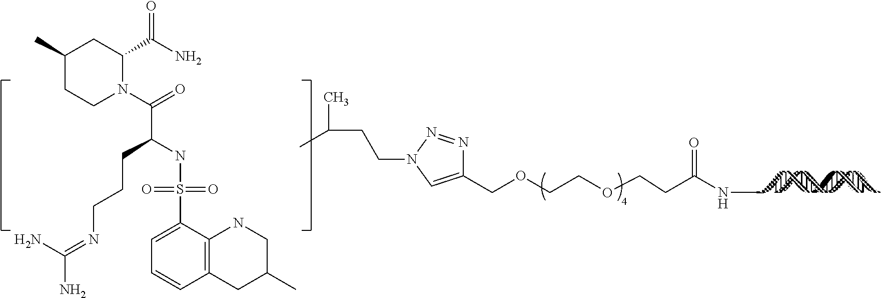 | 5871.39 | 5872.68 |
| Benazepril-HP- DNA | 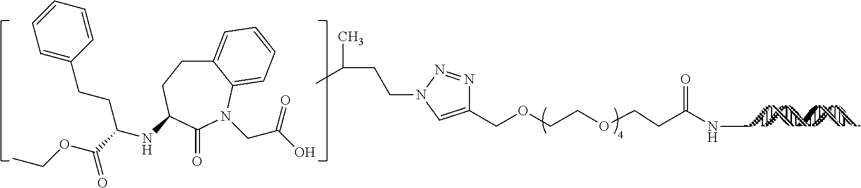 | 5788.33 | 5788.57 |
| Fenoprofen-HP- DNA | 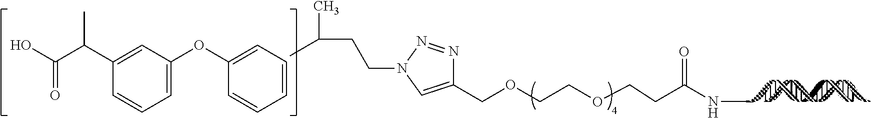 | 5606.22 | 5606.32 |
| Ciprofloxacin-HP- DNA | 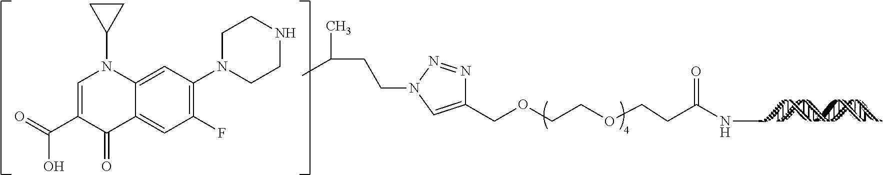 | 5695.26 | 5695.52 |
| Cinoxacin-HP- DNA | 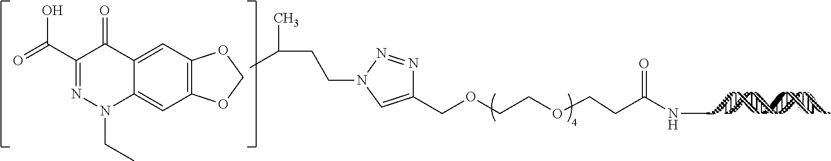 | 5626.19 | 5626.71 |
| Orbifloxacin-HP- DNA | 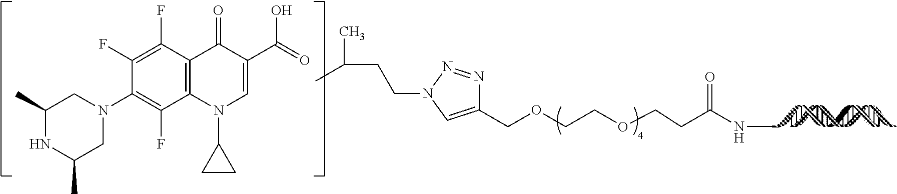 | 5759.28 | 5759.79 |
| Indoprofen-HP-DNA | 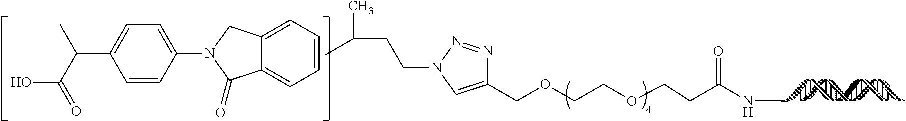 | 5645.24 | 5645.74 |
| Moxifloxacin-HP- DNA | 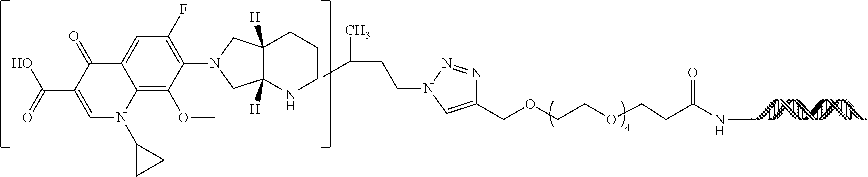 | 5765.31 | 5765.71 |
| Phthalylsulfathiazole- HP-DNA | 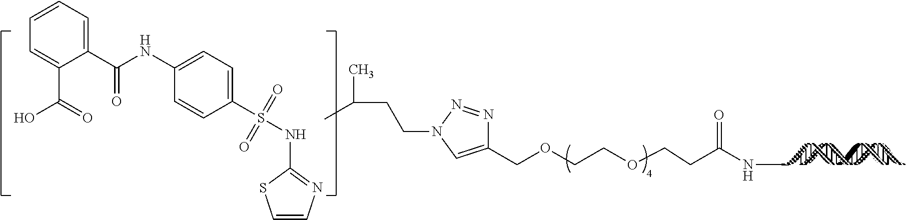 | 5767.16 | 5767.97 |
| Rabeprazole Related Compound E-HP-DNA | 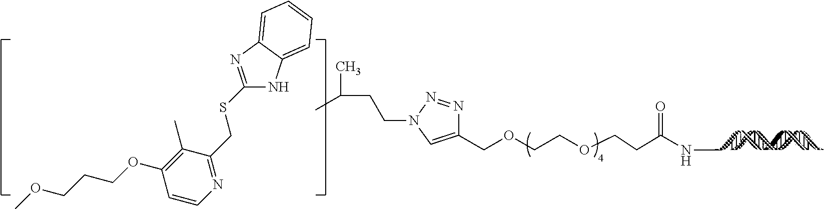 | 5707.27 | 5707.53 |
| Actarit-HP-DNA | 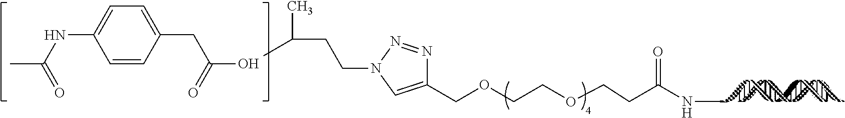 | 5557.20 | 5557.25 |
| Azilsartan-HP- DNA | 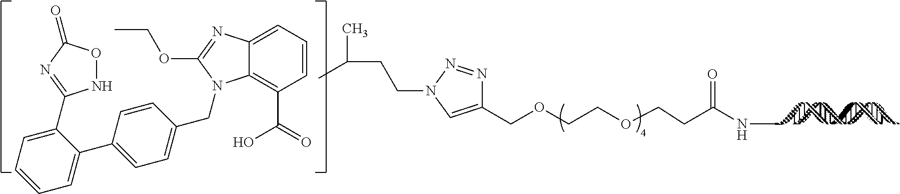 | 5820.27 | 5820.53 |
| Flumequine-HP- DNA | 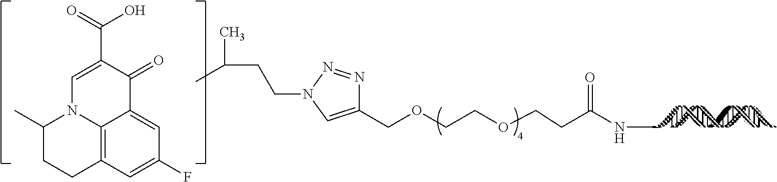 | 5625.21 | 5625.52 |
| Proglumide-HP- DNA | 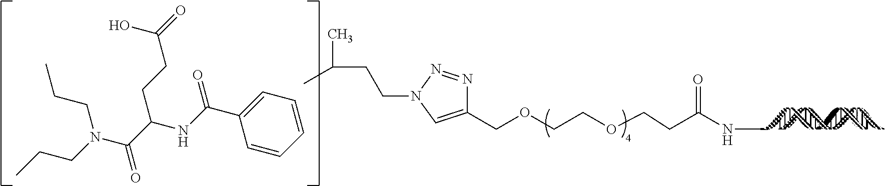 | 5698.32 | 5698.41 |
| Sparfloxacin-HP- DNA | 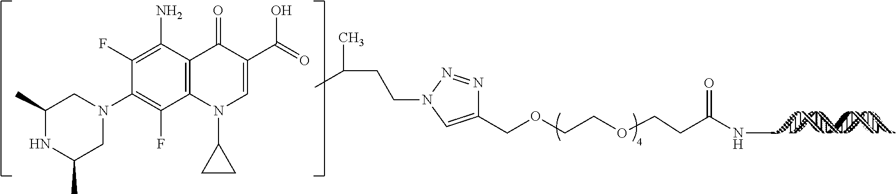 | 5756.30 | 5756.49 |
| Sarafloxacin-HP- DNA | 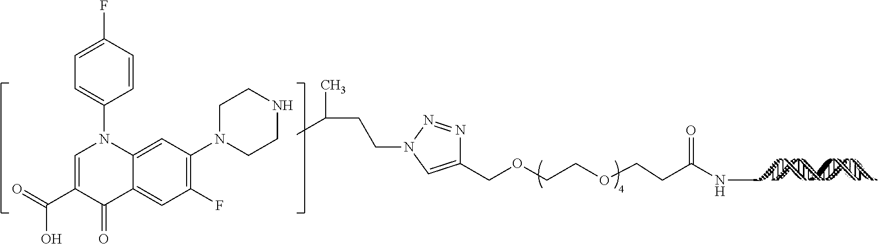 | 5749.25 | 5749.40 |
| Oxolinic acid-HP- DNA | 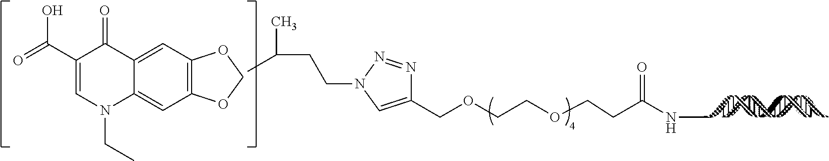 | 5625.19 | 5625.98 |
| Tazobactam acid- HP-DNA | 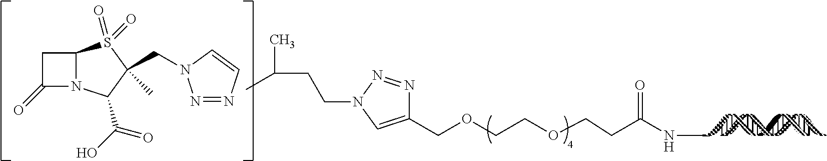 | 5664.18 | 5664.39 |
| Diacerein-HP-DNA | 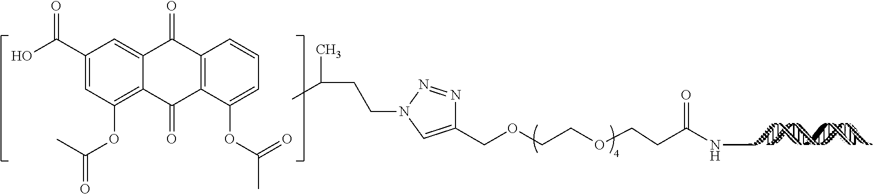 | 5732.18 | 5734.41 |
| Succinylsulfathiazole- HP-DNA | 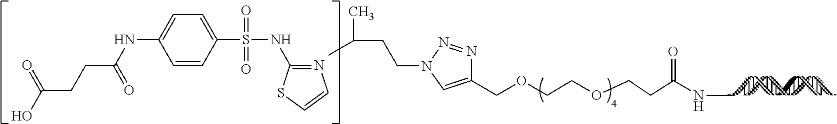 | 5719.16 | 5719.45 |
| Ofloxacin-HP-DNA | 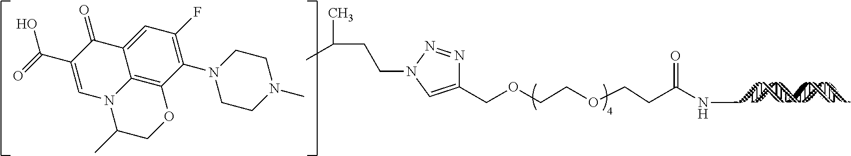 | 5725.27 | 5725.51 |
| Nalidixic acid-HP- DNA | 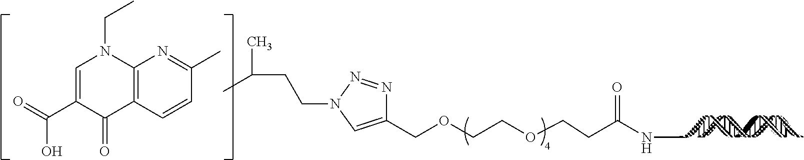 | 5596.21 | 5596.39 |
| Tetryzoline-HP- DNA | 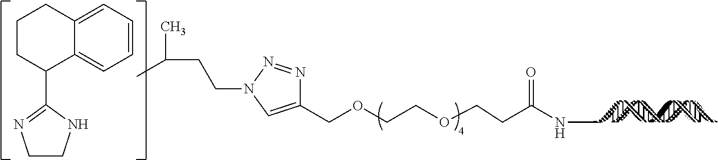 | 5564.26 | 5564.11 |
| Flubendazole-HP- DNA | 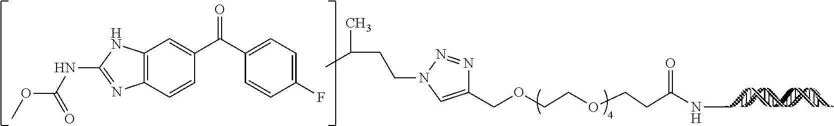 | 5677.22 | 5677.46 |
| Phenytoin-HP- DNA | 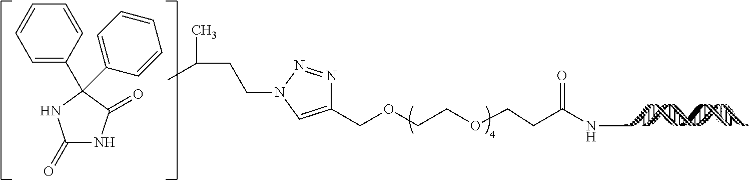 | 5616.22 | 5616.35 |
| Ziprasidone-HP- DNA | 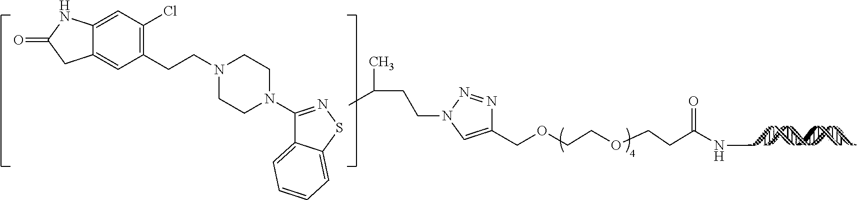 | 5776.24 | 5776.83 |
| Sulfalen-HP-DNA | 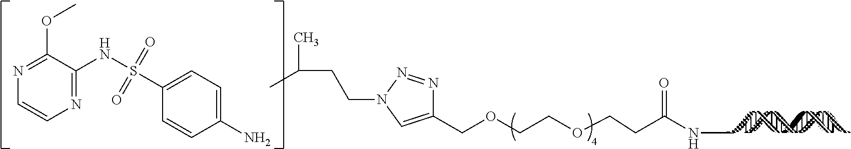 | 5644.19 | 5644.50 |
| Thalidomide-HP- DNA | 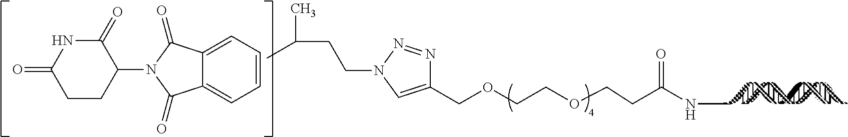 | 5622.19 | 5621.78 |
| Gemcitabine-HP-DNA | 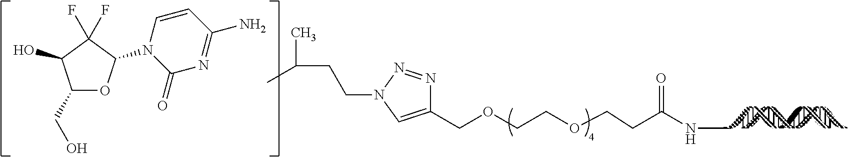 | 5627.20 | 5626.77 |
| Tizanidine-HP- DNA | 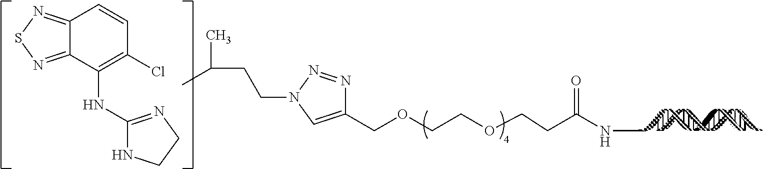 | 5617.15 | 5617.10 |
| Methylthiouracil- HP-DNA | 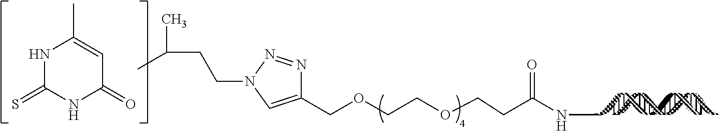 | 5506.15 | 5602.75 |
| Rosiglitazone-HP- DNA | 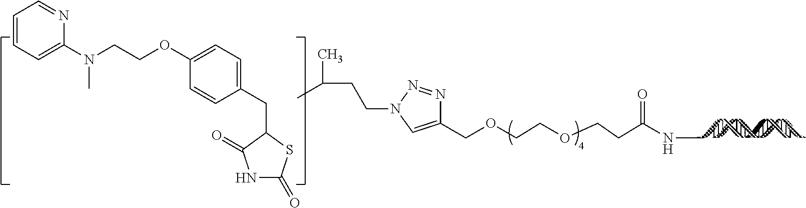 | 5721.24 | 5720.76 |
| Zileuton-HP-DNA | 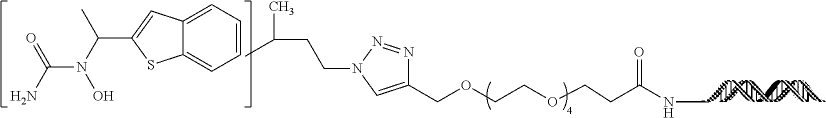 | 5600.19 | 5599.88 |
| Sunitinib-HP-DNA | 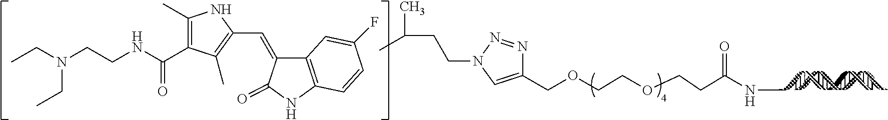 | 5762.34 | 5762.04 |
| Losartan-HP-DNA | 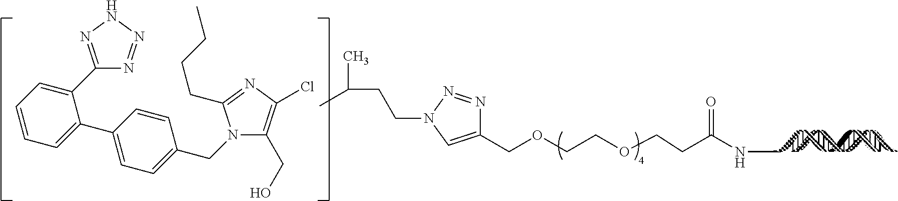 | 5786.29 | 5786.53 |
| Pioglitazone-HP- DNA | 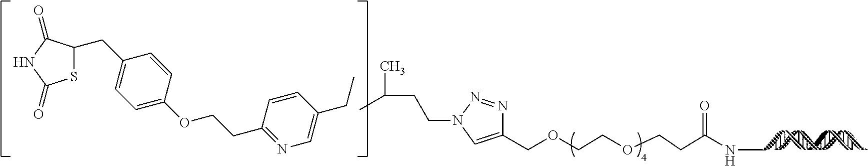 | 5720.25 | 5720.02 |
| Fluorocytosine-HP- DNA | 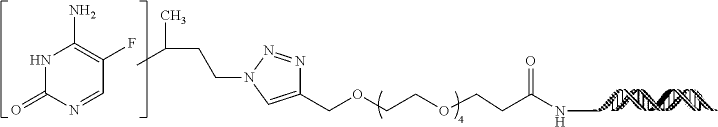 | 5493.16 | 5492.31 |
| R-(+)- Lansoprazole-HP- DNA | 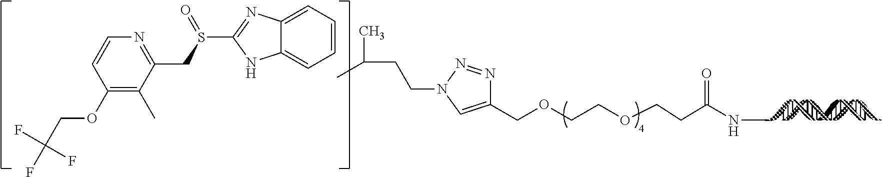 | 5733.21 | 5732.99 |
| Zolmitriptan-HP- DNA | 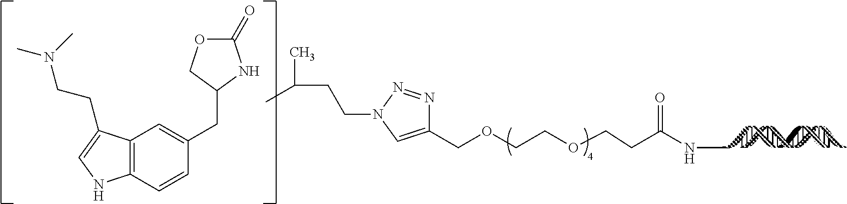 | 5651.29 | 5651.62 |
| Fenbendazole-HP- DNA | 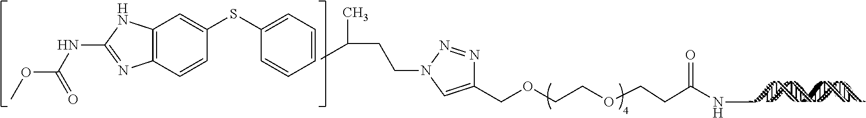 | 5663.20 | 5663.26 |
| Albendazole-HP- DNA | 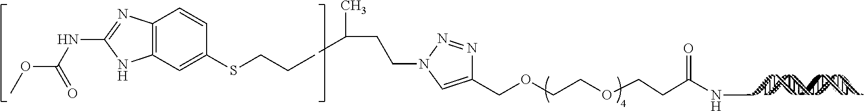 | 5629.22 | 5629.47 |
| Mebendazole-HP- DNA | 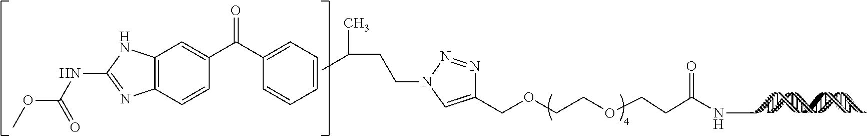 | 5659.23 | 5659.28 |
| Tolazamide-HP- DNA | 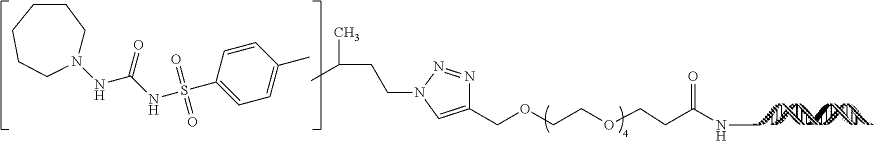 | 5675.26 | 5675.46 |
| Azathioprine-HP- DNA | 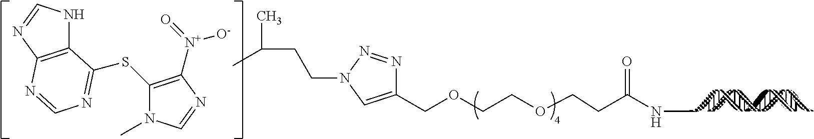 | 5641.17 | 5641.39 |
| Oxfendazole-HP- DNA | 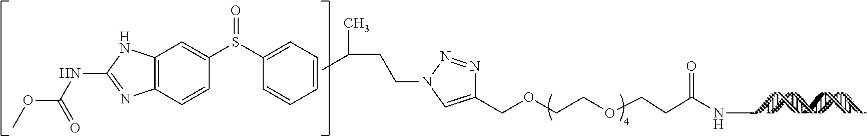 | 5679.20 | 5679.42 |
| Ganciclovir-HP- DNA | 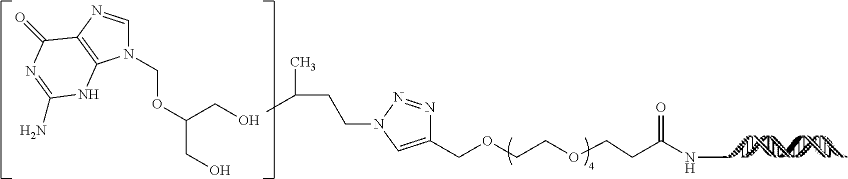 | 5619.23 | 5619.09 |
| Allopurinol-HP- DNA | 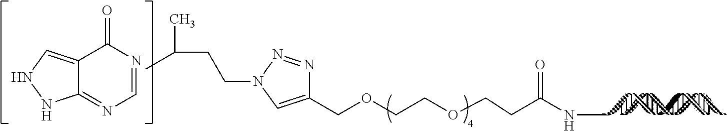 | 5500.17 | 5500.97 |
| Lansoprazole-HP- DNA | 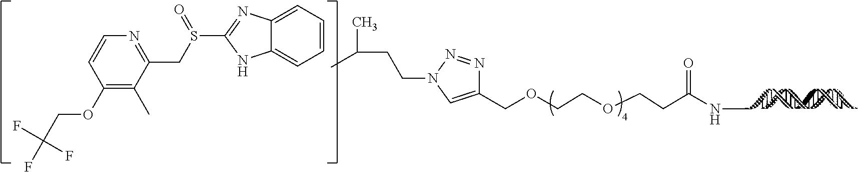 | 5733.21 | 5733.58 |
| Omeprazole-HP- DNA | 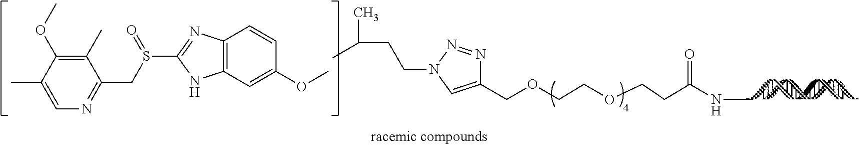 | 5709.24 | 5709.72 |
| Thiabendazole-HP- DNA | 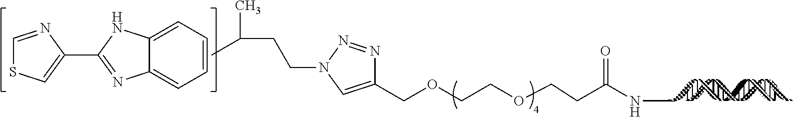 | 5565.17 | 5565.52 |
| Axitinib-HP-DNA | 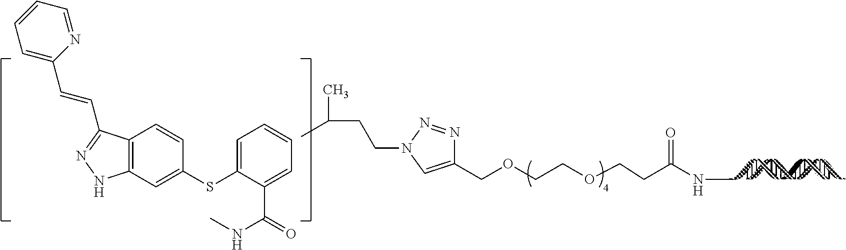 | 5750.25 | 5750.77 |
| Benicar-HP-DNA | 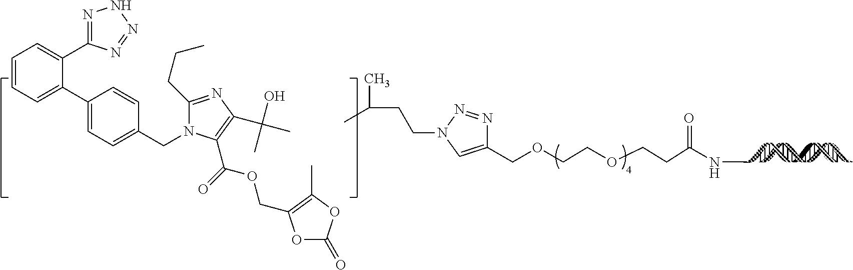 | 5922.35 | 5922.87 |
| Omeprazole sulfide-HP-DNA | 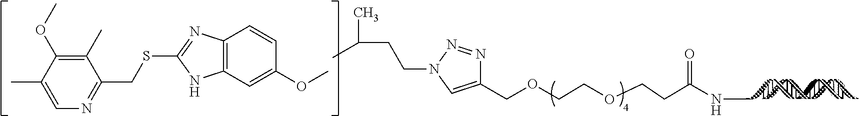 | 5693.25 | 5693.78 |
| Esomeprazole-HP- DNA | 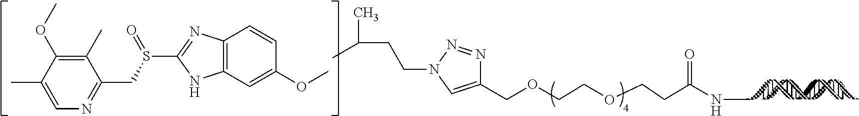 | 5709.24 | 5709.78 |
| Irbesartan-HP-DNA | 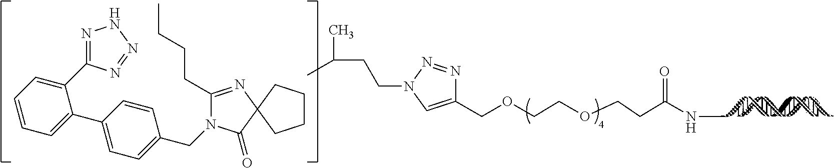 | 5792.36 | 5792.55 |
| Chlorzoxazone-HP- DNA | 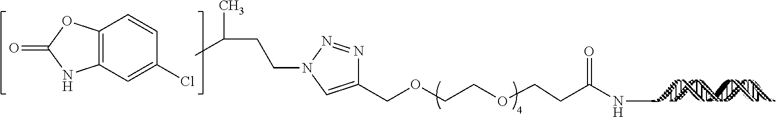 | 5533.12 | 5533.56 |
| Alizapride-HP- DNA | 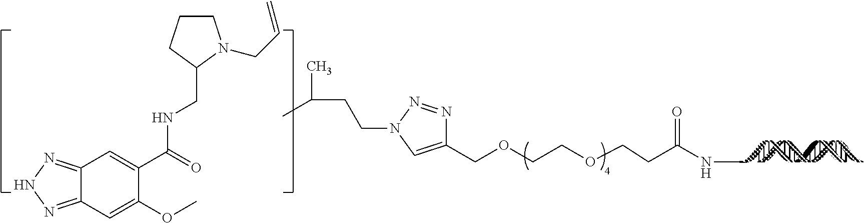 | 5679.30 | 5679.73 |
| Carbendazim-HP- DNA | 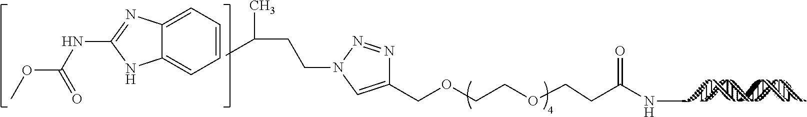 | 5555.20 | 5555.40 |
| Topiroxostat-HP- DNA | 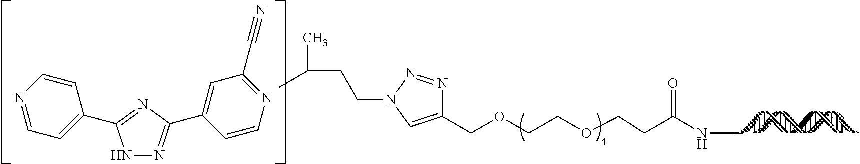 | 5612.21 | 5612.57 |
| CAL-101-HP-DNA | 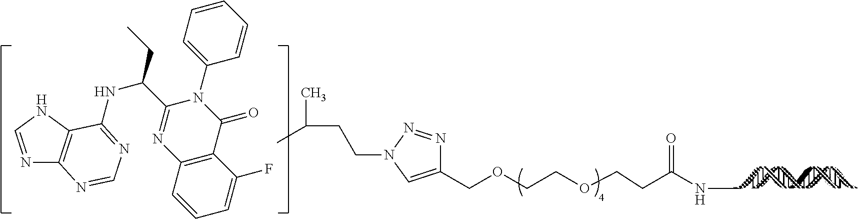 | 5779.29 | 5779.78 |

### Example 18: Combinatorial Synthesis Construction

To take advantage of the fact that a DNA encoded chemical library (DEL) can be screened in a single test tube, select DELs synthesized by late stage modification reactions were incorporated into a DEL library format for screening. To this end, both the late stage annotated DELs (including traditional Chinese Medicine natural products (TCMs), FDA approved drugs, and control compounds in clinical testing) and a small combinatorial DEL library of 10⁴ in size were prepared and then combined with a 1:10 ratio (single compound concentration). Using two known inhibitors of carbonic anhydrase II (CAII), carzenide and brinzolamide, the effect of different mixing ratios on enrichment of late stage labeled DELs was tested. The two CAII inhibitors were first DNA encoded, and spiked in the 10⁴ combinatorial DEL (0.5 pM/molecule) at final concentrations of 0.05 pM, 0.5 pM, and 5 pM. The mixing of 0.05 pM concentration of the late stage labeled DELs (1:10 ratio) showed the highest enrichment of 300- and 410-folds for brinzolamide and carzenide, respectively (Table 5).

TABLE 5
Enrichment folds for carbonic anhydrase binder selection

| | Concentration (pM) | | |
|---|---|---|---|
| Drug | 5 | 0.5 | 0.05 |
| carzenide | 30 | 32 | 410 |
| brinzolamide | 5.4 | 45 | 302 |

The amount of spiked natural product-DNA conjugation was quantified by quantitative polymerase-chain-reaction (qPCR) and then mixed with a DEL library with indicated ratio. The pilot DEL library contains 12696 compounds, which was constructed by the coupling of 6 amine-(PEG)n-acids (building block 1), 46 amino acids (building block 2), and 46 carboxylic acids (building block 3).

The DNA encoding framework was designed based on the structure of the headpiece and other barcodes in the literature. The headpiece is DNA headpiece (5′-/5 phos/GAGTCA/iSp9/iUniAmM/iSp9/TGACTCCC-3′). The DNA oligo-barcodes were enzymatically ligated in ligation buffer and T4 DNA ligase (NEB, Cat. #Z1811S). The reaction mixture was incubated at 16° C. for 16 h and analyzed by LCMS and gel. Sequencing primers are 5′-AATGATACGGCGACCACCGAGATCTACACTCTTTCCCTACACG (SEQ ID NO:2) and 5′-CAAGCAGAAGACGGCATACGAGATGTCGTGATGTGACTGGAGTTC (SEQ ID NO:3) and the general scheme is as shown in FIG. 19.

The his-tag fused recombinant human PARP-1 (Sino Biological, Cat. #11040-H08B) and human-HSP70 (Sino Biological, Cat. #11660-H07H) were obtained from commercial source. The panning procedure for these two soluble protein were the same. 5 μg of target protein was mixed with Ni-charged MagBeads (GenScript, Cat. #L00295), 5 nM DEL library and 10 μg/mL Salmon sperm DNA. The final volume to adjusted to 100 μL. The mixture was rotated at room temperature for 1.5 h. After washing 5 times by phosphate-buffered saline supplemented 0.05% Tween-20 (PBST), the target protein binding chemical-DNA conjugations were eluted by heating at 95° C. for 10 min in 50 μL elution buffer (20 mM Tris, pH 7.4, 100 mM NaCl). The eluted DEL compounds were amplified by PCR using Takara PrimerSTAR Max DNA Polymerase (Takara, Cat. #R045A). Then the excess primers was removed by Hieff NGS™ smarter DNA clean beads (Yeasen, Cat. #12600E503) and evaluated on 4% DNA agarose gel. The amplified products was subjected for high-throughput sequencing using Illumina HiSeq X10 Analyzer. The affinity selection and PCR amplification of oligonucleotide tags for different targets are summarized in Table 6.

TABLE 6

| | | HSP70¹ | | PARP-1² | |
|---|---|---|---|---|---|
| Affinity | target protein | 5 | μg | 5 | μg |
| selection | nDEL library | 5 | nM | 5 | nM |
| | salmon sperm DNA | 10 | μg/mL | 10 | μg/mL |

| protein buffer | PBS | PBS |
|---|---|---|
| affinity resin | Ni-charged MagBeads³ | |
| incubation | 1.5 h at room temperature | |
| wash buffer | PBST | PBST |

| | | |
|---|---|---|
| | washing | 5 times |
| | elution buffer | 20 mM Tris, pH 7.4 100 mM NaCl |
| | elution | heating at 95° C. for 10 min |
| PCR | polymerase | PrimeSTAR Max Premix (2X) ⁴ |
| amplification | primers | Sequencing primers, 10 pmol each |
| of oligo- | template | elution solution 10 μL |
| nucleotide | Sterilized | to final reaction volume of 50 μL |
| tags | distilled water | |

¹Sino Biological, Cat. #11660-H07H
²Sino Biological, Cat. #11040-H08B
³GenScript, Cat. #L00295
⁴ Takara, Cat. #R045A

The combinatorial chemical library was constructed by three building block sub libraries, containing 6, 46 and 46 chemical building blocks respectively for the first, second and third building block libraries, respectively. Each building block was encoded by a 10-base pair (bp) DNA sequence. The natural products were encoded by a 30-bp DNA sequence, same length as the combinatorial chemical library DNA codes. All the possible combinations (combinatorial chemical compounds) between these three sub libraries were generated by an inhouse java program, which generated a reference DNA encoding library contains 12696 DNA encoding sequences. After sequencing, the Illumina adaptors around the DNA coding sequences were trimmed by CLC genomics workbench version 12 (Qiagen). The left DNA coding sequences were 30 bp in length corresponding to the three DNA sequences of building blocks in 3 rounds of “split-pool” iterations. For each sample, the DNA coding sequences were mapped to the reference DNA encoding compound library. No mismatch was allowed in the mapping. The coding sequences were counted for all the compounds across different samples. The total read-counts of all the compounds in a given sample were calculated. The read-counts for each individual compound were divided by the total counts, multiplied with a constant of 100000.

NormalizedCount_{CompoundM,SampleA}=Count_{CompoundM,SampleA}/(TotalCount_{SampleA})*100000

The fold changes of each compound in the after-selection library comparing to the reference library were calculated. For example, if SampleA and Reference library were compared, for the fold change of CompoundM was calculated as:

FoldChange_{CompoundM}=NormalizedCount_{CompoundM,SampleA}/NormalizedCount_{CompoundM,Ref}

The hit criteria of nDEL screening take into account of both the normalized enrichment fold values (y-axis) and deep sequencing read counts (x-axis). Compounds with read counts less than 10 are considered unreliable, thus they are eliminated immediately from DEL before on-target screening. For each DEL library, a baseline enrichment fold is recorded in the absence of target protein, and a normalized enrichment fold value can be calculated for each DEL compound in the library. The cutoff for hits identification is based on a simplified statistical analysis of a highly diverse population of data, which is the sum of average value of enrichment-folds of the whole library (μ) plus 3 times of the standard deviation (σ). Any DEL compounds showing enrichment-fold greater than μ+3σ are considered hits.

### PARP-1 Enzymatic Assays.

PARP-1 autoribosylation based assay (BPS, Cat. #80580) were carried out following the provided protocols. Compounds were dissolved in DMSO. Experimental reactions were set up in triplicate by pre-incubation of the proteins with compounds in a vary range depend on different compounds (final concentration of DMSO was 1% in all samples) for 15 min at room temperature. ADP-ribosylation reactions were then prepared by two-fold dilution into substrate coated assay plates and incubated at room temperature for 1 h. Chemiluminescence was detected using a micoplate reader (EnVision, PerkinElmer). The resulting data were fitted to a single-site dose-response model using GraphPad to extract experimental IC₅₀ values. The reported errors represent the standard error of the fitted parameter for each experiment.

### Molecular Modeling

Molecular docking in silico was used to investigate the possible binding modes of luteolin on the cataliytical domain of PARP1 (residues 660 to 1011). AutoDock Tools (version 4.2.6) was used for PARP1 (PDB 4pjt) and ligands preparation to generate pdbqt files. Water molecules and inhibitor were removed from PARP1 PDB file, polar hydrogens and Gasteiger partial charges were added. A grid of 60×60×40 points in x,y,z axes and a space of 0.375 Å was centered on the inhibitor binding site. A total of 200 runs were performed with a maximal number of 2 500 000 energy evaluations. Luteolin binding mode with lowest free energy of binding was selected for further molecular dynamics simulations.

Parameters and topology for molecular dynamics simulation for the Luteolin molecule were derived by ANTECHAMBER software and ACPYPE script using the semi-empirical quantum chemistry program (SQM) and the Generalized Amber Force Field (GAFF). The luteolin-PARP1 complex was subjected to a short energy minimization, followed by 100-ns molecular dynamics simulation to relax the interaction and stabilize the structure of the complex. Simulations were performed using the Gromacs 4.6.7 package and the Amber 14ffSB force field. The system was solvated with full-atom TIP3P water containing Cl⁺ and K⁺ ions at a concentration of 0.13 M to mimic a physiological ionic strength. Temperature T and pressure P were kept constant at 300 K and 1 atm, respectively, using the Berendsen thermostat and barostat. Fast smooth Particle-Mesh Ewald summation was used for long-range electrostatic interactions, with a cutoff of 1.0 nm for the direct interactions. It is considered that PARP-1 residues interact stably with Leutolin if their distance with the chemical is below a cut-off of 3.0 angstrom for more than 90% of the time along the molecular dynamics trajectory. These residues are listed in the Table 7.

TABLE 7
Residues of PARP-1 interacting with Luteolin
in the MD simulation trajectory.

| Residue name | Interaction probability along |
|---|---|
| and index | the trajectory (100 ns) |
| GLU 988 | 99.9 |
| ALA 898 | 97.7 |
| TYR 896 | 97.4 |
| ASP 766 | 96.2 |
| HIS 862 | 93.6 |
| GLY 863 | 87.2 |
| TYR 889 | 84.5 |
| GLU 763 | 76 |
| TYR 907 | 74.8 |
| VAL 762 | 68.3 |
| LYS 903 | 66.6 |
| SER 904 | 64.8 |
| GLN 759 | 54.4 |
| GLY 888 | 45.2 |
| PHE 897 | 37.4 |
| TRP 861 | 13 |
| MET 890 | 12 |
| ALA 880 | 2.4 |
| ASN 767 | 0.4 |

Screening of nDEL Against Heat-Shock 70 kDa (HSP70) Protein and Poly[ADP-Ribose]Polymerase I (PARP1)

Two target proteins with different cellular locations and biophysical properties were used in nDEL screens. These include HSP70 in cytosol and PARP1 in nucleus, each of them possesses a different affinity preference for small molecule ligands. The known functional binders for these targets were included in nDEL as internal positive controls, e.g., oridonin for HSP70 and derivatives of olaparib (F001, F002, F003 and F006) for PARP-1. To account for the non-uniform distribution of DNA barcodes in nDEL, a blank screen of nDEL in the absence of target proteins but presence of immobilization matrix beads was carried out first and followed by deep-sequencing to establish the baseline distribution of barcodes in the library. All screening data were analyzed using the method described by Decurtins et al., Nat Protoc. 2016; 11(4): 764-80. The corresponding DNA sequence of each nDEL compound were tallied based on its sequencing counts. The enrichment fold is calculated as the ratio of normalized sequencing counts in the presence and absence of target protein. Enrichment folds are shown in the Tables below.

TABLE 8A
Enrichment folds for HSP70 binder selection

| Drug (NP) | Enrichment Fold |
|---|---|
| Scopoletin | 6.0 |
| F002 | 5.8 |
| Oridonin-A1 | 5.3 |
| (−)-Epicatechin gallate | 5.0 |
| Berbamine dihydrochloride | 4.8 |
| Naringin | 4.5 |
| Jatrorrhizine hydrochloride | 4.5 |
| Gentisic acid | 4.5 |
| Plumbagin | 4.4 |
| Alizarin | 4.3 |
| Epigallocatechin | 4.3 |
| Theobromine | 4.3 |
| 7-Hydroxycoumarin | 4.2 |
| Esculetin | 4.1 |
| Picroside II | 4.0 |
| Daphnetin | 3.9 |
| Liquiritin | 3.9 |
| Nocodazole | 3.9 |
| Luteolin | 3.8 |
| Phlorizin | 3.8 |
| Protocatechuic acid | 3.8 |
| (+)-Catechin Hydrate | 3.8 |
| Jatrorrhizine | 3.7 |
| Biochanin A | 3.7 |
| Methyl protocatechuate | 3.6 |
| Baicalein | 3.6 |
| F006 | 3.6 |
| Benzyladenine | 3.6 |
| Bengenin | 3.6 |
| Phloracetophenone | 3.5 |
| Pyridoxine | 3.4 |
| Hyperoside | 3.4 |
| Theophylline-7-acetic acid | 3.3 |
| F001 | 3.3 |
| Fraxetin | 3.3 |
| sinomenine | 3.2 |
| Synephrine | 3.2 |
| rutaecarpin | 3.2 |
| Danirixin | 2.9 |
| Kinetin | 2.8 |
| quercetin | 2.8 |
| (+) Catechin | 2.7 |
| Liquiritigenin | 2.7 |
| Artesunate | 2.4 |
| Oridonin-B | 2.3 |

TABLE 8B
Enrichment folds for PARP-1 binder selection

| Drug (NP) | Fold Change |
|---|---|
| F001 | 4.38 |
| F002 | 3.69 |
| Oridonin-A1 | 3.28 |
| F006 | 3.21 |
| Plumbagin | 2.97 |
| Scopoletin | 2.82 |
| (−)-Epicatechin gallate | 2.81 |
| Liquiritin | 2.66 |
| Epigallocatechin | 2.53 |
| Theobromine | 2.42 |
| F003 | 2.34 |
| Hyperoside | 2.25 |
| Jatrorrhizine hydrochloride | 2.23 |
| Gentisic acid | 2.21 |
| Naringin | 2.17 |
| Luteolin | 2.17 |
| Daphnetin | 2.17 |
| Synephrine | 2.17 |
| Berbamine dihydrochloride | 2.15 |
| Nocodazole | 2.06 |
| Jatrorrhizine | 2.04 |
| Alizarin | 2.02 |
| Fraxetin | 2.01 |

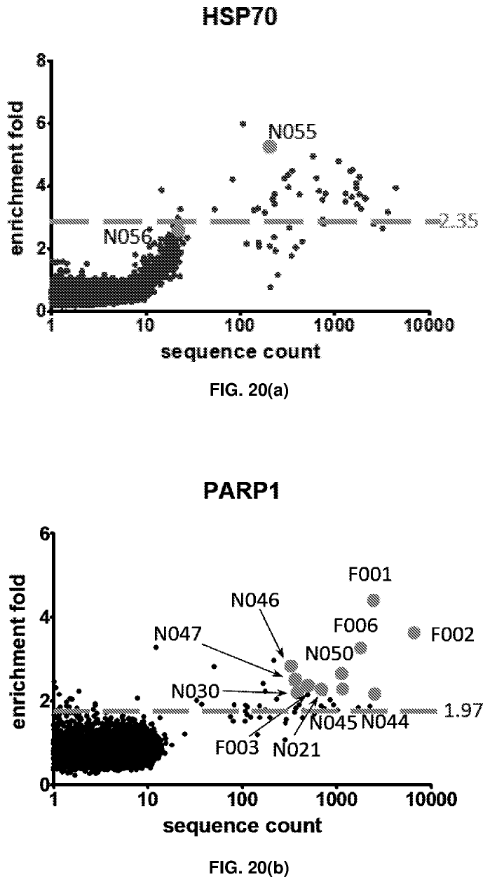

The screening finger-print of nDEL was plotted as enrichment fold vs. normalized sequencing counts as shown in FIG. 20. Using known binders as internal references, hits of nDEL screening were identified based on the enrichment folds of positive control compounds.

The nDEL screening was performed against the purified human proteins of HSP70 and PARP-1. The affinity captured nDELs were subject to deep-sequencing and decoding analysis. Results are summarized in Table 9. All control compounds were enriched in nDEL screening, and the hit rate ranging from 0.15% to 0.47% (FIG. 20). The observed higher hit rate for HSP70 may be associated with the stickiness of HSP70 protein. The first two fractions were collected and coded with two unique DNA sequences N055 and N056. Notably the two stereo-isomers of ordorion labelled compounds N055 and N056 were enriched 5.3- and 2.3-fold, respectively (FIG. 20(a) and Table 8A), indicative structural preferences of HSP70 towards different stereoisomers.

TABLE 9
DEL screening summary

| Target name | Hits (number) | Hit rates |
|---|---|---|
| HSP70 | 60 | 0.47% |
| PARP1 | 34 | 0.27% |

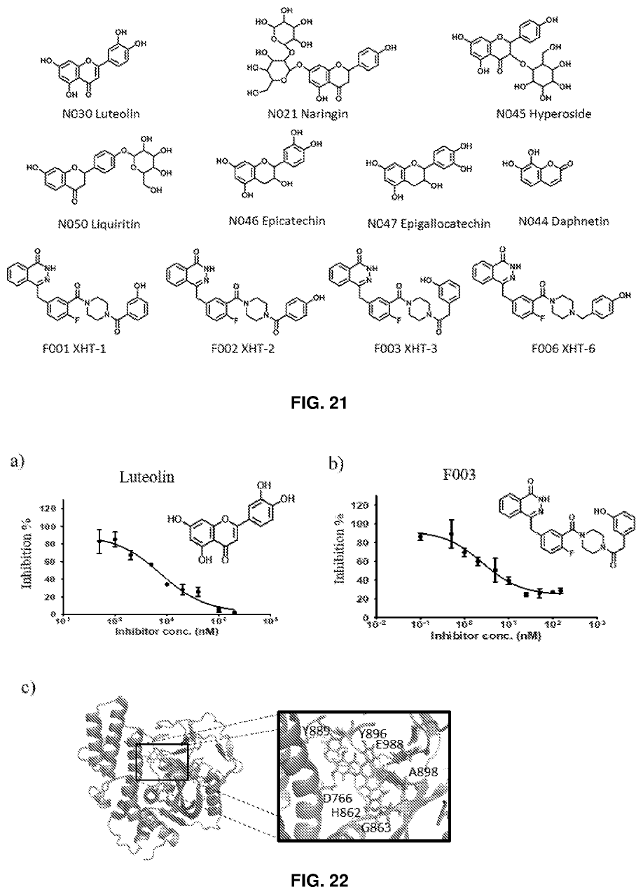

In the PARP-1 screening, 34 nDELs were enriched (FIG. 20(b)), 4 of which including the positive control compounds were confirmed (FIG. 21). The internal control with known compounds in nDEL appeared to greatly enable the selection of real positive hits. Interestingly, flavonoids with similar structures were clustered in the enriched chemicals, in particular, a TCM compound, luteolin and its glycosylate analogues naringin and hyperoside were also selected.

Biochemical Characterization of nDEL Hits in PARP-1 Enzyme Inhibition

PARP-1, a validated target in cancer therapy, catalyzes rapid transfer of ADP-ribose fragment from NAD⁺ to acceptor protein, and itself resulting in formation of protein-bound linear and branched homo-ADP-ribose polymers in response to cellular signals of DNA damage and repair. The enzyme activity was measured based on auto-ribosylation of PARP-1 in the presence of sheared DNA.

Inhibitory activities of enriched nDELs were characterized by PARP-1 auto-ribosylation assay. A derivative of the positive control olaparib, F003, showed potent PARP-1 inhibition with an IC₅₀ value of 2.5 nM (FIG. 22(b)). A TCM nDEL, Luteolin, inhibited the enzyme activity of PARP-1 with an IC₅₀ value of 7.5 μM (FIG. 22(a)). In order to understand the interaction between PARP-1 and Luteolin, a series of in silico analysis based on molecular modeling were performed. By using molecular docking it was found that, in the lowest free energy binding mode, Luteolin occupied the catalytic domain of PARP-1. To further assess the stability of this mode and understand the molecular details of interaction, a 100 ns molecular dynamics simulations were performed, starting from the predicted docking model. The complex appeared stable in the simulated time windows and several residues in the protein interacted for a substantial time period with Luteolin. In particular D766, H862, Y896 and E988 kept their contacts with Luteolin for over 90 percent of the simulated trajectory. Luteolin in the catalytic site of PARP-1 appears to be stabilized due to the hydrogen bonds formed with side chains of G863, E988 and D766 residues, which are also the key residues in NAD binding (FIG. 22(c)).

### Discussion

DELs were synthesized using combinatorial methods including split-pool synthesis, which fundamentally is an iterative process requiring multiple complex transformations in the presence of DNA. Compounds with highly complex steric structures such as natural products are usually not included in DELs because their synthesis requires more sophisticated chemical transformations. The relative advantages of nature's selection over time vs. DELs selection using large numbers has yet to be determined. However, for the first time, the current study provides a way to study both systems simultaneously in a single test tube. Enabling DEL screens under identical environmental conditions provided more insight into different DELs and their applications. Moreover, in nDELs the known binders or inhibitors of target proteins could serve as internal controls, which greatly improves the confirmation rate and hit selection in DEL screens. Importantly, natural products may have evolved toward a single objective and may not be useful for other goals. The use of nDELs overcomes this limitation by exposing the target to a vast collection of potential ligands.

A special feature of the protocol described herein is the use of volatile linkers between DNA and organic compounds. It has been demonstrated that incomplete chemical synthesis and undesired by-products can compromise DEL screens (e.g., excess linker may react with the biological target). A volatile linker allows easy removal of unreacted linker molecules so that multiple reactions can be run in a single sample without concern regarding modification of the linkers. Therefore, the subsequent analysis is not confounded by linker modifications. The labeling efficiency of diazirine was found to be low for certain compounds, especially for those C—H only chemicals, because carbene insertion into C—H bonds of many natural products can be problematic. New late stage modification methods are necessary to expand the nDEL approach. The C—H insertion of nitrene and carbon radical generated by 4-((trimethylsilyl)ethynyl) phenyl sulfamate and sodium 7-azido-1,1-difluoroheptane-1-sulfinate showed great promise as complementary tools. These modifications could also serve as alternative labeling methods to generate additional geometric isomers in compounds with single functional groups.

Despite its early stage of development, the nDEL with limited numbers already showed encouraging potential in hit identification for targets of different categories. The discovery of Luteolin as an inhibitor of the PARP-1 enzyme highlights the power of nDELs. Luteolin is a natural flavonoid found in many fruits and vegetables such as carrots, broccoli, onion leaves, parsley, celery, sweet bell peppers, and chrysanthemum flower. Luteolin is also an active ingredient in many medical herbs in traditional Chinese medicine such as Lonicera japonica, chrysanthemum, Herba unripe, Prunella vulgaris, artichoke, perilla, Scutellaria, and purple flower, etc. Traditionally, these herbs were used in complex formulas as an anti-inflammatory to relieve cough, reduce phlegm, and to treat diseases, such as angiocardiopathy and hepatitis. Luteolin has been extensively studied due to its potent anti-cancer activity against a wide spectrum of cancer cell types. More importantly, it showed efficacy in reversing the growth of multi-drug resistant cancer cells (MDR). Luteolin is believed to exert its anticancer activities via apoptosis and cell cycle regulation. Multiple molecular targets e.g. JNK, NF-κB, IGF-1, etc. have been suggested for Luteolin. However, evidence of direct interactions with a defined binding pocket is still lacking for any proposed target. Moreover, one or all of the listed targets can not reconcile all the pharmacologic behaviors of Luteolin. Polypharmacology, a common frustration in the study of natural products, greatly limits the clinical development of these active natural compounds. The identification of PARP-1 for Luteolin by nDEL screening demonstrates the potential of nDELs in polypharmacology dissection of natural products. Poly(ADP-ribose) polymerase 1 (PARP-1) binds to DNA in response to transient and localized DNA strand breaks in cells caused by a variety of biological processes including DNA repair, replication, recombination, and gene rearrangement.

As a clinically proven chemotherapeutic target, PARP-1 inhibition displayed similar patterns of regulation in apoptosis, cell cycle arrest, etc. as those observed for Luteolin. It seems likely PARP could be one of the key targets of Luteolin, which orchestrates the polypharmacologic effects. nDELs, with the potential of integrating numbers, diversities, and information, could be invaluable in our efforts to find cures and solutions to biomedical problems.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A linker precursor molecule C comprising a first functional group and a second functional group, wherein the first functional group is capable of generating a carbene or nitrene or free-radical that is capable of reacting with an organic compound A, and the second functional group is unreactive under the conditions when the first functional group reacts with the organic compound A, but is capable of reacting with a second organic compound B.

2. The linker precursor molecule C of claim 1 which is of the formula R-L-M, wherein R is the first functional group, M is the second functional group, and L is a linker moiety comprising one or more of moieties independently selected from optionally substituted alkylene, optionally substituted alkenylene, optionally substituted alkynylene, optionally substituted heteroalkylene, optionally substituted cycloalkylene, optionally substituted heterocycloalkylene, optionally substituted arylene, and optionally substituted heteroarylene.

3. The linker precursor molecule C of claim 1, wherein the first functional group comprises a diazirine, aryl azide, benzophenone, or diazo.

4. The linker precursor molecule C of claim 1, wherein the first functional group is selected from the group consisting of 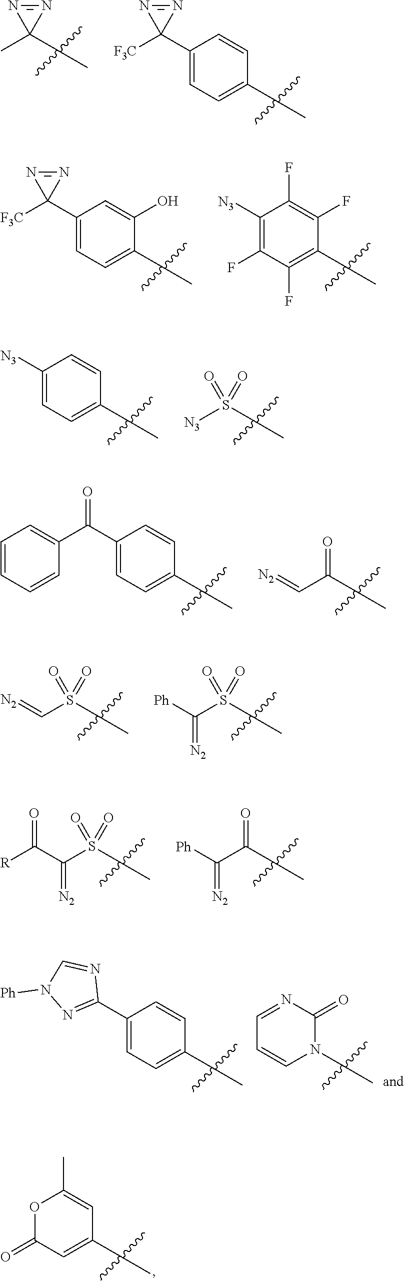
wherein [symbol] represents the point of attachment to the rest of the linker precursor molecule C.

5. The linker precursor molecule C of claim 1, wherein the second functional comprises an azide, alkyne, ene, or —C(O)—O—.

6. The linker precursor molecule C of claim 1, wherein the second functional group is selected from the group consisting of 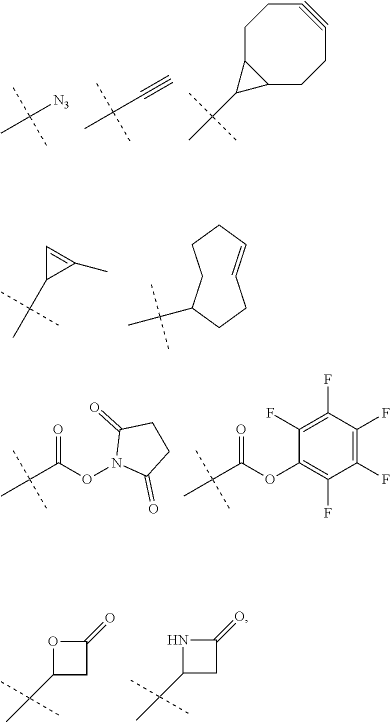
wherein [symbol] represents the point of attachment to the rest of the linker precursor molecule.

7. The linker precursor molecule C of claim 1, wherein L is an alkylene wherein one or more of the methylene units of the alkylene is optionally replaced with a moiety independently selected from the group consisting of NR¹, O, S, CO, SO, SO₂, NR¹C(O), C(O)NR¹, cycloalkylene, heterocycloalkylene, arylene, and heteroarylene, wherein R¹ is H or alkyl.

8. The linker precursor molecule C of claim 1, wherein L is an alkylene wherein one or more of the methylene units of the alkylene is replaced with a moiety independently selected from O, NHC(O) and C(O)NH.

9. The linker precursor molecule C of claim 1, which is selected from the group consisting of

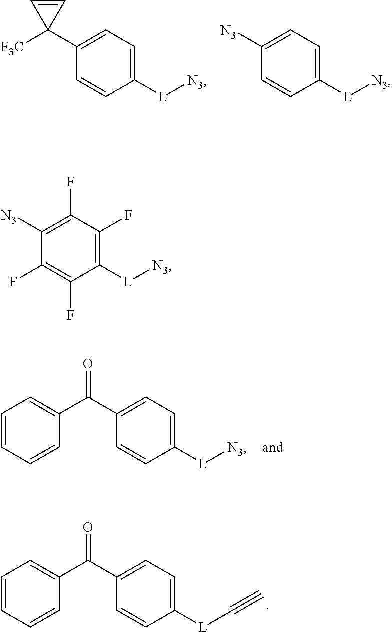

10. The linker precursor molecule C of claim 1, which is selected from the group consisting of

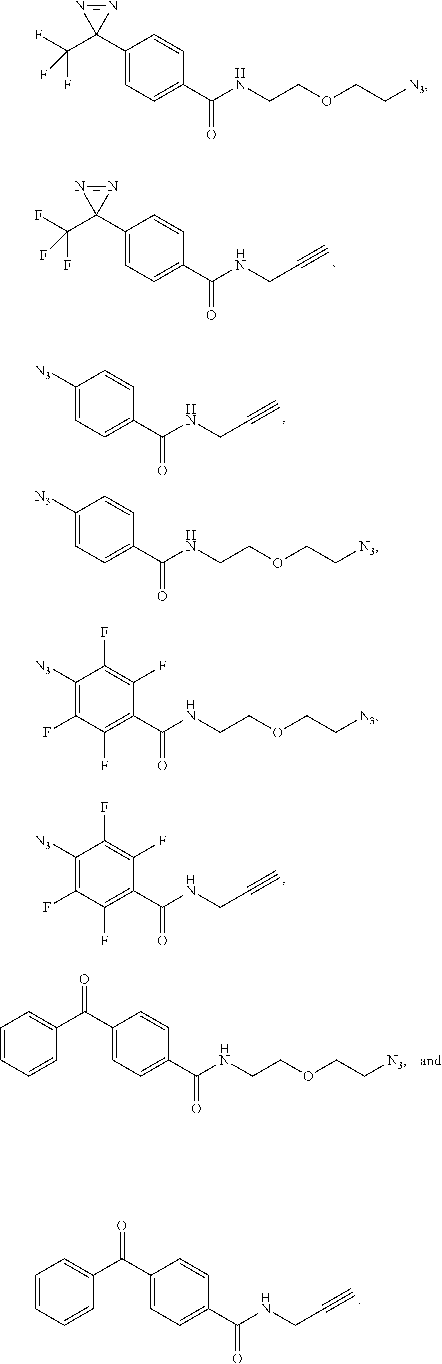

11. (canceled)

12. A labeled organic compound E which is produced by a method comprising:
(1) reacting the first functional group of the linker precursor molecule C of claim 1 with an organic compound A under carbene or nitrene or free-radical reaction conditions to form an intermediate D having the second functional group of the linker precursor molecule C of claim 1; and
(2) reacting the second functional group with a labeling molecule B comprising a label.

13. The labeled organic compound E of claim 12, wherein the organic compound A does not comprise a reactive functionality.

14. The labeled organic compound E of claim 12, wherein the label comprises a unique sequence, or a fluorescent tag, or a combination thereof.

15. The labeled organic compound E of claim 14, wherein the unique sequence comprises a single-strand DNA or RNA, a double-strand DNA or RNA, a triplex DNA or RNA, a multi-strand natural or artificial oligonucleotide or a chemically modified oligonucleotide.

16. The labeled organic compound E of claim 13, wherein the unique sequence comprises an oligonucleotide.

17-18. (canceled)

19. A mixture comprising (1) a plurality of isomeric compounds wherein the isomeric compounds are produced by carbene or nitrene or free radical reaction of an organic compound A with a linker precursor molecule C of claim 1, and/or (2) one or more pairs of compounds that are products of a disproportionation reaction of a compound D produced by the carbene or nitrene or free radical reaction of the organic compound A with the linker precursor molecule C.

20-21. (canceled)

22. A mixture of a plurality of isomeric labeled compounds which is produced by a method comprising:
(1) reacting the first functional group of the linker precursor molecule C of claim 1 with an organic compound A under carbene or nitrene or free-radical reaction conditions to form an intermediate D having the second functional group of the linker precursor molecule of claim 1; and
(2) reacting the second functional group with a labeling molecule B comprising a label.

23-25. (canceled)

26. A method of labeling an organic compound A comprising
(1) reacting the first functional group of the linker precursor molecule C of claim 1 with the organic compound A under carbene or nitrene or free-radical reaction conditions to form an intermediate D having the second functional group of the linker precursor molecule of claim 1; and
(2) reacting the second functional group with a labeling molecule B comprising a label.

27-36. (canceled)

37. A method for identifying a compound having two conjugated double bonds, the method comprises
(1) reacting the first functional group of the linker precursor molecule C of claim 1 with an organic compound A under carbene or nitrene or free-radical reaction conditions to form a product or a mixture of products wherein the second functional group of the linker precursor molecule remains unreacted; and
(2) analyzing the product or mixture of products, wherein the presence of a pair of disproportionation reaction products indicates that the organic compound A has two conjugated double bonds, wherein the pair of disproportionation reaction products comprise a compound F having a molecular weight that is the molecular weight of a product D of the carbene or nitrene or free-radical reaction plus 2, and a compound F′ having a molecular weight that is the molecular weight of the product D of the carbene or nitrene or free-radical reaction minus 2.

38. A method for treating a cancer, stroke, myocardial infarction, a neurodegenerative disease or inflammation in a subject in need thereof, comprising administering to the subject a therapeutically effecting amount of luteolin, naringin, hyeroside, liquiritin, epicatechin, epigallocatechin, daphnetin, F001, F002, F003 or F006, or a derivative thereof.

39-42. (canceled)
